SEQUENCE LISTING (50 sequences)

SEQ ID NO: 1 - protein, 171 aa, synthetic construct
MFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEK

SEQ ID NO: 2 - protein, 9 aa, synthetic construct
  VARIANT 4: note = G or S
  VARIANT 5: note = P, L, or R
GRGXXGRKW

SEQ ID NO: 3 - protein, 72 aa, synthetic construct
ENLYFQGSFKEFKGKIESKMLYLGEEVKLLGEGKITGKLVGLSEKGGALILTEEGIKEILSGEFSLRRSGGS

SEQ ID NO: 4 - protein, 4 aa, synthetic construct
GGGS

SEQ ID NO: 5 - protein, 5 aa, synthetic construct
GGGGG

SEQ ID NO: 6 - protein, 6 aa, synthetic construct
GSGSGS

SEQ ID NO: 7 - protein, 6 aa, synthetic construct
GGSGGS

SEQ ID NO: 8 - protein, 5 aa, synthetic construct
GGGGS

SEQ ID NO: 9 - protein, 16 aa, synthetic construct
GGGGSLVPRGSGGGGS

SEQ ID NO: 10 - protein, 11 aa, synthetic construct
GGSGGHMGSGG

SEQ ID NO: 11 - protein, 17 aa, synthetic construct
VEGGSGGSGGSGGSGGV

SEQ ID NO: 12 - protein, 18 aa, synthetic construct
  VARIANT 8: note = Any amino acid
GSTSGSGXPGSGEGSTKG

SEQ ID NO: 13 - protein, 233 aa, Aquifex sp.
MFKNLIWLKEVDSTQERLKEWNVSYGTALVADRQTKGRGRLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILTEEGIKEILSGEFSLRRS

SEQ ID NO: 14 - protein, 9 aa, synthetic construct
  VARIANT 5: note = L or P
GRGRXGRKW

SEQ ID NO: 15 - protein, 12 aa, synthetic construct
GGGSGGGSGGGS

SEQ ID NO: 16 - protein, 9 aa, synthetic construct
  VARIANT 4: note = G or S
  VARIANT 5: note = Any amino acid
GRGXXGRKW

SEQ ID NO: 17 - protein, 233 aa, synthetic construct
MFKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILTEEGIKEILSGEFSLRRS

SEQ ID NO: 18 - DNA, 699 bp, synthetic construct
  source 1..699: mol_type = other DNA
atgtttaagaatctaatatggttaaaagaagtggatagcacccaggagcgcctgaaagagtggaatgtgagctatggtactgctttagtcgcagatcgccagaccaaaggtagaggcgggttgggccgtaaatggctgtcgcaagagggaggcctgtacttctcctttctgttgaacccgaaagagttcgaaaacctgttacaactgccgctggttctaggtctctcagtttctgaggccctggaagaaattaccgaaatcccgtttagcctgaagtggccaaacgacgtctatttccaagagaaaaaagtttctggcgtactttgcgaactgtctaaggacaaattgatcgtgggcattggtattaacgtgaatcagcgtgaaatcccggaagagatcaaggaccgcgcgaccaccctgtacgagatcacgggcaaggactgggatcgtaaagaagtgctgttgaaggttctcaagcgtattagcgagaacctgaaaaagttcaaagagaagagctttaaagagtttaagggtaagatcgaaagcaagatgctgtacttgggcgaggaggttaaactgctcggtgaaggcaagattacgggtaaattggtgggtctgagcgaaaagggtggcgcgctgatcttgaccgaagaaggtattaaggagatcctgtccggcgagttctccctgcgtcgtagc

SEQ ID NO: 19 - protein, 171 aa, synthetic construct
MFKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEK

SEQ ID NO: 20 - DNA, 513 bp, synthetic construct
  source 1..513: mol_type = other DNA
atgtttaagaatctaatatggttaaaagaagtggacagcacccaagaacgtctgaaggagtggaacgttagctatggtactgctctggttgcggatcgccagaccaaaggtcgtggtggcctgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaa

SEQ ID NO: 21 - DNA, 513 bp, synthetic construct
  source 1..513: mol_type = other DNA
atgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaa

SEQ ID NO: 22 - protein, 302 aa, synthetic construct
MVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGGSGGGSGGGSMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEK

SEQ ID NO: 23 - DNA, 906 bp, synthetic construct
  source 1..906: mol_type = other DNA
atggtaactacactatcaggattatctggcgagcagggtccgagcggcgatatgaccactgaggaagactctgccacgcatatcaaattttcgaagcgcgacgaagacggtcgtgaactggcgggtgctacgatggaattgcgtgatagcagcggtaagaccatttccacctggattagcgacggccacgtgaaagacttctacctgtatccaggtaaatatacctttgttgaaaccgcggcaccggatggctacgaggtggcgactccgatcgagttcacggttaacgaagatggtcaagtaacagtagatggtgaagctacagaaggtgatgcacatacaggttcaagtggaagtggaggtggcagtggcggcggcagtggtggaggcagtatgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaa

SEQ ID NO: 24 - protein, 266 aa, synthetic construct
EVQLVQSGAELVKPGASVKLSCKVSDYNIRRTYMHWVRQRPGKGLEWIGRIDPANGNTIYGEKFKSKATLTADTSSNTAYMQLSQLKSDDTAIYYCAIGVQYLDYWGQGTTVTVSSGGGGSGGGGSGGGGSDIVLTQSPALAVSPGERVTISCRATESVSTLIHWYQQRPGQQPKLLIYLTSHLDSGVPARFSGSGSGTDFTLTIDPVEADDTATYYCQQTWNDPWTFGGGTKLELKRGGGSGGGSGGGSRGVPHIVMVDAYKRYK

SEQ ID NO: 25 - DNA, 798 bp, synthetic construct
  source 1..798: mol_type = other DNA
gaggttcagctagtacaatcaggagctgaattggttaagccgggcgcttccgtcaagctgagctgcaaagtttcggactataacattcgccgtacctacatgcattgggttcgtcagcgtccgggtaagggtttggaatggattggtcgtattgatccggctaatggaaacaccatctacggcgagaaattcaaaagcaaagcgaccctgactgcggacacctctagcaataccgcgtatatgcagctgtctcagcttaagagcgatgacacggcgatttattactgcgcaatcggcgtgcagtatctcgactactggggtcaaggtacgaccgtgaccgtctccagcggcggcggtggctctggtggcggtggctccggtggtggcggttccgatattgtactgacgcagagcccggcgctggccgtttccccgggtgaacgtgttaccatcagctgtcgcgcaactgagagcgtgtcgaccctgatccactggtatcagcaaagaccaggtcaacaaccgaaattgttaatctacctgaccagccacctggacagcggggtgccggcccgttttagcggttctggcagcggcaccgatttcaccctgaccatcgatccggtggaagcagacgacactgcgacgtactactgccagcaaacctggaacgatccttggacctttggtggtggtacaaaattggagctgaagcgcggaggtggcagtggcggcggcagtggtggaggcagtaggggagttccccacatagtaatggtcgatgcgtataagcgctataag

SEQ ID NO: 26 - protein, 255 aa, synthetic construct
MATLQESEVKVDGEQKLSKNELKRRLKAEKKLAEKEAKQKELSEKQLNQTASAPNHTADNGVGAEEETLDDDDDDSGENLYFQGMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEK

SEQ ID NO: 27 - DNA, 763 bp, synthetic construct
  source 1..763: mol_type = other DNA
ggctacactacaagagtcagaagtaaaagtggatggcgaacagaagttgtccaaaaacgaattaaagcgccgtctgaaagcggagaagaaattggctgagaaggaggccaaacaaaaagaactgtctgaaaagcaactgaatcagaccgcgagcgcaccgaaccacaccgcggataatggcgttggtgcggaggaagagacgctggatgacgacgacgatgacagcggtgagaacctgtacttccagggaatgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaa

SEQ ID NO: 28 - protein, 373 aa, synthetic construct
MVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGGSGGGSGGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 29 - DNA, 1119 bp, synthetic construct
  source 1..1119: mol_type = other DNA
atggtaactacactatcaggattatctggcgagcagggtccgagcggcgatatgaccactgaggaagactctgccacgcatatcaaattttcgaagcgcgacgaagacggtcgtgaactggcgggtgctacgatggaattgcgtgatagcagcggtaagaccatttccacctggattagcgacggccacgtgaaagacttctacctgtatccaggtaaatatacctttgttgaaaccgcggcaccggatggctacgaggtggcgactccgatcgagttcacggttaacgaagatggtcaagtaacagtagatggtgaagctacagaaggtgatgcacatacaggttcaagtggaagtggaggtggcagtggcggcggcagtggtggaggcagtggagaatcactatttaaagggcccagggattacaatccgattagctccaccatctgccacctgaccaacgagagcgacggccacaccacttccttatacggcatcggctttggtccgttcattatcaccaacaagcacttgtttcgccgtaacaatggtacattgttggtccagagcttgcatggtgttttcaaagttaagaacaccacgacgctgcaacaacacctgattgatggccgtgatatgatcatcatcagaatgccgaaggacttcccgcctttcccgcagaaactgaaatttcgtgagccgcagcgtgaagagcgcatttgcctggtcaccaccaacttccagacgaaaagcatgtctagcatggtttccgatacctcatgtacctttccgagctcggacggcatcttttggaaacattggattcagaccaaggacggccaatgcggttccccgctcgtatctactcgcgacggcttcatcgtgggtattcatagcgcaagcaatttcaccaataccaacaactatttcacgtctgtgccaaagaactttatggaacttctgaccaaccaagaagcgcagcaatgggttagcggttggcgtctgaatgctgatagcgtgctgtggggtggtcacaaagtgttcatgtcgaaaccggaagaaccgtttcaaccggtgaaggaggcgacccagctgatgaacgagctggtttatagccag

SEQ ID NO: 30 - protein, 381 aa, synthetic construct
MHHHHHHMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGGSGGGSGGGSMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKENLYFQGSFKEFKGKIESKMLYLGEEVKLLGEGKITGKLVGLSEKGGALILTEEGIKEILSGEFSLRRSGGS

SEQ ID NO: 31 - DNA, 1122 bp, synthetic construct
  source 1..1122: mol_type = other DNA
atggtaactacactatcaggattatctggcgagcagggtccgagcggcgatatgaccactgaggaagactctgccacgcatatcaaattttcgaagcgcgacgaagacggtcgtgaactggcgggtgctacgatggaattgcgtgatagcagcggtaagaccatttccacctggattagcgacggccacgtgaaagacttctacctgtatccaggtaaatatacctttgttgaaaccgcggcaccggatggctacgaggtggcgactccgatcgagttcacggttaacgaagatggtcaagtaacagtagatggtgaagctacagaaggtgatgcacatacaggttcaagtggaagtggaggtggcagtggcggcggcagtggtggaggcagtatgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaagaaaatctatattttcaaggatcatttaaagaattcaagggcaagatcgaaagcaaaatgctgtacctgggcgaggaagtgaaactgctgggtgagggcaagatcacgggtaagttggttggtctctctgaaaaaggcggtgcgctgattttgaccgaagagggtattaaagagatcttatccggcgagttcagcctgcgccgtagcggtggttcg

SEQ ID NO: 32 - protein, 378 aa, synthetic construct
MVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGGSGGGSGGGSMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKENLYFQGGGGSSFKEFKGKIESKMLYLGEEVKLLGEGKITGKLVGLSEKGGALILTEEGIKEILSGEFSLRRSGGS

SEQ ID NO: 33 - DNA, 1134 bp, synthetic construct
  source 1..1134: mol_type = other DNA
atggtaactacactatcaggattatctggcgagcagggtccgagcggcgatatgaccactgaggaagactctgccacgcatatcaaattttcgaagcgcgacgaagacggtcgtgaactggcgggtgctacgatggaattgcgtgatagcagcggtaagaccatttccacctggattagcgacggccacgtgaaagacttctacctgtatccaggtaaatatacctttgttgaaaccgcggcaccggatggctacgaggtggcgactccgatcgagttcacggttaacgaagatggtcaagtaacagtagatggtgaagctacagaaggtgatgcacatacaggttcaagtggaagtggaggtggcagtggcggcggcagtggtggaggcagtatgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaagaaaatctatattttcaaggaggaggtggcagttcatttaaagaattcaagggcaagatcgaaagcaaaatgctgtacctgggcgaggaagtgaaactgctgggtgagggcaagatcacgggtaagttggttggtctctctgaaaaaggcggtgcgctgattttgaccgaagagggtattaaagagatcttatccggcgagttcagcctgcgccgtagcggtggttcg

SEQ ID NO: 34 - protein, 251 aa, synthetic construct
MFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKENLYFQGGGGSGGGSSFKEFKGKIESKMLYLGEEVKLLGEGKITGKLVGLSEKGGALILTEEGIKEILSGEFSLRRSGGS

SEQ ID NO: 35 - DNA, 753 bp, synthetic construct
  source 1..753: mol_type = other DNA
atgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaagaaaatctatattttcaaggaggaggtggcagtggcggcggcagttcatttaaagaattcaagggcaagatcgaaagcaaaatgctgtacctgggcgaggaagtgaaactgctgggtgagggcaagatcacgggtaagttggttggtctctctgaaaaaggcggtgcgctgattttgaccgaagagggtattaaagagatcttatccggcgagttcagcctgcgccgtagcggtggttcg

SEQ ID NO: 36 - protein, 393 aa, synthetic construct
MHHHHHHMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGGSGGGSGGGSMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKENLYFQGGGGSGGGSGGGSSFKEFKGKIESKMLYLGEEVKLLGEGKITGKLVGLSEKGGALILTEEGIKEILSGEFSLRRSGGS

SEQ ID NO: 37 - DNA, 1158 bp, synthetic construct
  source 1..1158: mol_type = other DNA
atggtaactacactatcaggattatctggcgagcagggtccgagcggcgatatgaccactgaggaagactctgccacgcatatcaaattttcgaagcgcgacgaagacggtcgtgaactggcgggtgctacgatggaattgcgtgatagcagcggtaagaccatttccacctggattagcgacggccacgtgaaagacttctacctgtatccaggtaaatatacctttgttgaaaccgcggcaccggatggctacgaggtggcgactccgatcgagttcacggttaacgaagatggtcaagtaacagtagatggtgaagctacagaaggtgatgcacatacaggttcaagtggaagtggaggtggcagtggcggcggcagtggtggaggcagtatgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaagaaaatctatattttcaaggaggaggtggcagtggcggcggcagtggtggaggcagttcatttaaagaattcaagggcaagatcgaaagcaaaatgctgtacctgggcgaggaagtgaaactgctgggtgagggcaagatcacgggtaagttggttggtctctctgaaaaaggcggtgcgctgattttgaccgaagagggtattaaagagatcttatccggcgagttcagcctgcgccgtagcggtggttcg

SEQ ID NO: 38 - protein, 312 aa, synthetic construct
QVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVTVSSGGLPETGGGGGSGGGSGGGSMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEK

SEQ ID NO: 39 - DNA, 936 bp, synthetic construct
  source 1..936: mol_type = other DNA
caagttcagctagtagaaagtggtggagggttggtagagccgggtggcagcctgcgtctgagctgtgctgcgtctggcattatctttaaaatcaatgatatgggttggtatcgccaagcaccaggtaagcgtagagaatgggttgcagcttccaccggcggtgatgaggcgatttaccgcgacagcgtgaaggaccgcttcaccattagccgtgacgccaagaactccgtgttcttgcaaatgaacagcctgaaaccggaggacaccgcggtgtactactgcactgcggtcatctcgaccgatcgtgatggtacggaatggcgtcgttattggggtcagggtacgcaggttaccgtttctagcggcggcctgccggaaaccggtggcggaggtggcagtggcggcggcagtggtggaggcagtatgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaa

SEQ ID NO: 40 - protein, 308 aa, synthetic construct
QVQLQESGGGSVQAGGSLRLSCAASGYTYSSYCMGWFRQAPGKEREGVAAIYPDAGITFYTDSVKGRFTISRDNAKNTLFLQMNSLKPEDTATYYCAAAPPSVPCRLVVARYNYWGQGTQVTVSSGGGSGGGSGGGSMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEK

SEQ ID NO: 41 - DNA, 924 bp, synthetic construct
  source 1..924: mol_type = other DNA
caagtacagctacaagaatcaggtggagggtcggtccaggcgggcggttctctgcgtctgagctgcgcggcgtccggttatacgtacagctcctattgcatgggttggttccgccaggctccgggtaaagagcgcgagggcgtggctgccatctacccggacgcgggcatcaccttttatactgacagcgtgaagggccgtttcaccattagccgtgataatgcaaaaaacaccctgtttctgcaaatgaacagcttgaagccggaagataccgcaacctactactgcgcggctgcgccaccgtccgttccgtgtagattggttgtggcccgttataactactggggtcaaggtacgcaggttaccgtaagctctggaggtggcagtggcggcggcagtggtggaggcagtatgtttaagaatctaatatggttaaaagaagtggacagcacccaagaacgtctgaaggagtggaacgttagctatggtactgctctggttgcggatcgccagaccaaaggtcgtggtggcccgggccgtaagtggctgtcgcaagaaggtggcctctacttcagctttctgttaaatccgaaagagtttgaaaacttgctgcagctgccgctggtcttgggtttgtccgtgtctgaggcgctggaagagatcacggaaatcccgttttctctgaagtggccaaatgatgtttatttccaagagaagaaggtcagcggtgttctttgcgaactgtccaaagacaaactgatcgtgggcatcggcattaacgtgaaccagcgtgaaatcccggaagagattaaagaccgcgcaaccaccctgtacgaaattaccggtaaagactgggatcgtaaggaggttttgttgaaggtgctcaagcgcattagcgagaacctgaagaaattcaaagagaaa

SEQ ID NO: 42 - protein, 308 aa, synthetic construct
MVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGSGGGSGGGSGGGSMFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKHHHHHH

SEQ ID NO: 43 - protein, 40 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 6: note = Any amino acid
  VARIANT 8: note = Any amino acid
  VARIANT 9: note = Any amino acid
  VARIANT 11: note = Any amino acid
  VARIANT 12: note = Any amino acid
  VARIANT 14: note = Any amino acid
  VARIANT 15: note = Any amino acid
  VARIANT 20: note = Any amino acid
  VARIANT 25: note = Any amino acid
  VARIANT 27: note = Any amino acid
  VARIANT 28: note = Any amino acid
  VARIANT 30: note = Any amino acid
  VARIANT 31: note = Any amino acid
  VARIANT 34: note = Any amino acid
  VARIANT 36: note = Any amino acid
  VARIANT 37: note = Any amino acid
  VARIANT 38: note = Any amino acid
  VARIANT 39: note = Any amino acid
  VARIANT 40: note = Any amino acid
XXXXGXAXXAXXQXXGRGRXGRKWXSXXGXXLYXSXXXXX

SEQ ID NO: 44 - protein, 39 aa, Aquifex sp.
NVSYGTALVADRQTKGRGRLGRKWLSQEGGLYFSFLLNP

SEQ ID NO: 45 - protein, 40 aa, Escherichia coli
ELKSGDACIAEYQQAGRGRRGRKWFSPFGANLYLSMFWRL

SEQ ID NO: 46 - protein, 236 aa, synthetic construct
MFKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILTEEGIKEILSGEFSLRRSGGS

SEQ ID NO: 47 - protein, 243 aa, synthetic construct
MFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKENLYFQGSFKEFKGKIESKMLYLGEEVKLLGEGKITGKLVGLSEKGGALILTEEGIKEILSGEFSLRRSGGS

SEQ ID NO: 48 - protein, 247 aa, synthetic construct
MFKNLIWLKEVDSTQERLKEWNVSYGTALVADRQTKGRGGPGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKENLYFQGGGGSSFKEFKGKIESKMLYLGEEVKLLGEGKITGKLVGLSEKGGALILTEEGIKEILSGEFSLRRSGGS

SEQ ID NO: 49 - protein, 8 aa, synthetic construct
GGGSGGGS

SEQ ID NO: 50 - protein, 21 aa, synthetic construct
  SITE 1: note = Dibenzocyclooctyne azide modified azidoornithine
XGGGSRGVPHIVMVDAYKRYK

(19) United States
(12) Patent Application Publication
(10) Pub. No.: US 2025/0223583 A1
(43) Pub. Date: Jul. 10, 2025

(54) AN ENZYMATIC SYSTEM FOR PRECISE CELL TARGETING

(71) Applicants: The Broad Institute, Inc., Cambridge, MA (US); The General Hospital Corporation, Boston, MA (US)

(72) Inventors: Rebecca Holden, Cambridge, MA (US); Matt Bakalar, Cambridge, MA (US); Nir Hacohen, Boston, MA (US)

(73) Assignees: The Broad Institute, Inc., Cambridge, MA (US); The General Hospital Corporation, Boston, MA (US)

(21) Appl. No.: 19/084,412

(22) Filed: Mar. 19, 2025

Related U.S. Application Data
(63) Continuation of application No. PCT/US2023/074780, filed on Sep. 21, 2023.
(60) Provisional application No. 63/408,726, filed on Sep. 21, 2022.

(51) Int. Cl. C12N 9/00; A61K 47/64; C12N 15/62; G01N 33/532

(52) U.S. Cl. CPC C12N 9/93; A61K 47/64; C12N 15/62; G01N 33/532; C07K 2319/50; C07K 2319/70; C12Y 603/04015

(57) ABSTRACT

The disclosure provides compositions and methods for enzyme-mediated precise cell targeting.

### CROSS-REFERENCE TO RELATED APPLICATIONS

This application is a continuation under 35 U.S.C. § 111 (a) of PCT International Patent Application No. PCT/US2023/074780, filed Sep. 21, 2023, designating the United States and published in English, which claims priority to and the benefit of U.S. Provisional Application No. 63/408,726, filed Sep. 21, 2022, the entire contents of each of which are incorporated by reference herein.

### STATEMENT OF RIGHTS TO INVENTIONS MADE UNDER FEDERALLY SPONSORED RESEARCH

This invention was made with government support under grant Nos. 5P01AI148102-02 and R01CA208756-05 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Oct. 6, 2023, is named “167741-049402US_SL.xml” and is 71,780 bytes in size.

### BACKGROUND OF THE INVENTION

Selectively targeting distinct sub-populations of cells remains a challenge across cancer immunity, cancer immunotherapy, and autoimmune diseases or disorders. Related subsets of cells with overlapping surface marker expression can exhibit different activities, necessitating greater precision than is possible with conventional approaches that use a single targeting moiety. Two-target approaches, such as bispecific antibodies and AND-gated CAR-Ts, seek to introduce higher selectivity, but still face limitations balancing activity and toxicity. There is a need for techniques to efficiently target cell sub-populations with high sensitivity and precision. Further, there is a crucial need for straightforward, generalizable approaches to pinpoint subsets of therapeutically relevant immune cells, such as those involved in cancer immunity or autoimmune diseases.

### SUMMARY OF THE INVENTION

The present disclosure provides compositions and methods for enzyme-mediated precise cell targeting.

In an aspect, the present disclosure provides a fusion polypeptide including a biotin protein ligase which includes an active site that has at least 85% amino acid sequence identity to the following sequence: GRGX₁X₂GRKW (SEQ ID NO: 2), having biotin ligase activity, and a targeting moiety fused to the biotin protein ligase polypeptide or fragment thereof at the N-or C-terminus. X₁ is G or S, and X₂ is P, L, or R.

In another aspect, the present disclosure provides a polynucleotide encoding the fusion polypeptide of any of the above aspects, or embodiments thereof.

In another aspect, the present disclosure provides a method of targeting a cell type of interest. The method involves contacting the cell with the fusion polypeptide of any one of the above aspects, or embodiments thereof, under conditions that support ligase activity.

In another aspect, the present disclosure provides a method of labeling a cell. The method involves contacting the cell with a biotin binding moiety covalently linked to a detectable moiety and a fusion polypeptide under conditions that support ligase activity, thereby labeling the cell. The fusion polypeptide includes: i) an agent that specifically binds the cell; and ii) a biotin protein ligase including an active site that has at least 85% amino acid sequence identity to the following sequence: GRGX₁X₂GRKW (SEQ ID NO: 2) and having biotin ligase activity. X₁ is G or S, and X₂ is P, L, or R.

In another aspect, the present disclosure provides a method of delivering an agent to a cell. The method involves contacting a cell with a fusion polypeptide of any one of the above aspects, or embodiments thereof, under conditions that support ligase activity, and a biotin binding moiety covalently linked to an agent, thereby delivering the agent to the cell.

In another aspect, the present disclosure provides a method for producing the fusion protein of any one of the above aspects, or embodiments thereof. The method involves expressing the fusion protein in a cell.

In another aspect, the present disclosure provides a kit including the fusion polypeptide of any of the above aspects, or embodiments thereof, and directions for the use of the kit and the methods described herein.

In another aspect, the present disclosure provides a system for use in delivering an agent to a cell of interest. The system includes a fusion protein including a biotin protein ligase fused to a targeting moiety, biotin, ATP, and a biotin binding moiety fused to an agent for delivery to the cell.

In any of the above aspects, or embodiments thereof, the active site has at least 90% amino acid identity to the sequence GRGX₁X₂GRKW (SEQ ID NO: 2). In any of the above aspects, or embodiments thereof, the active site has at least 95% amino acid identity to the sequence GRGX₁X₂GRKW (SEQ ID NO: 2). In any of the above aspects, or embodiments thereof, the active site has at least 97% amino acid identity to the sequence GRGX₁X₂GRKW (SEQ ID NO: 2). In any of the above aspects, or embodiments thereof, the active site includes the sequence GRGX₁X₂GRKW (SEQ ID NO: 2).

In any of the above aspects, or embodiments thereof, the biotin protein ligase has at least 85% amino acid sequence identity to MFKNLIWLKEVDSTQERLKEWNVSYGTALVADRQTKGRGGPGRKWLSQEGGLYF SFLLNPKEFENLLQLPLVLGLSVSEALEEITEIPFSLKWPNDVYFQEKKVSGVLCELSK DKLIVGIGINVNQREIPEEIKDRATTLYEITGKDWDRKEVLLKVLKRISENLKKFKEK (SEQ ID NO: 1). In any of the above aspects, or embodiments thereof, the biotin protein ligase has at least 90% amino acid sequence identity to MFKNLIWLKEVDSTQERLKEWNVSYGTALVADRQTKGRGGPGRKWLSQEGGLYF SFLLNPKEFENLLQLPLVLGLSVSEALEEITEIPFSLKWPNDVYFQEKKVSGVLCELSK DKLIVGIGINVNQREIPEEIKDRATTLYEITGKDWDRKEVLLKVLKRISENLKKFKEK (SEQ ID NO: 1). In any of the above aspects, or embodiments thereof, the biotin protein ligase has at least 95% amino acid sequence identity to MFKNLIWLKEVDSTQERLKEWNVSYGTALVADRQTKGRGGPGRKWLSQEGGLYF SFLLNPKEFENLLQLPLVLGLSVSEALEEITEIPFSLKWPNDVYFQEKKVSGVLCELSK DKLIVGIGINVNQREIPEEIKDRATTLYEITGKDWDRKEVLLKVLKRISENLKKFKEK (SEQ ID NO: 1). In any of the above aspects, or embodiments thereof, the biotin protein ligase includes or consists essentially of

(SEQ ID NO: 1)
MFKNLIWLKEVDSTQERLKEWNVSYGTALVADRQTKGRGGPGRKW
LSQEGGLYFSFLLNPKEFENLLQLPLVLGLSVSEALEEITEIPFS
LKWPNDVYFQEKKVSGVLCELSKDKLIVGIGINVNQREIPEEIKD
RATTLYEITGKDWDRKEVLLKVLKRISENLKKFKEK.

In any of the above aspects, or embodiments thereof, the biotin protein ligase is derived from a eukaryote or prokaryote. In any of the above aspects, or embodiments thereof, the biotin protein ligase is derived from a bacteria, plant, or mammal. In any of the above aspects, or embodiments thereof, the variant polypeptide is derived from E. coli, or A. aeolicus.

In any of the above aspects, or embodiments thereof, the targeting moiety is a polynucleotide, polypeptide, or small molecule. In any of the above aspects, or embodiments thereof, the targeting moiety specifically binds a polypeptide on the surface of a target cell. In any of the above aspects, or embodiments thereof, the target cell is a neoplastic cell or an immune cell. In any of the above aspects, or embodiments thereof, the targeting moiety is an antibody, nanobody, or antigen binding fragment thereof. In any of the above aspects, or embodiments thereof, the targeting moiety is an antibody, or antigen binding fragment thereof.

In any of the above aspects, or embodiments thereof, the antibody, nanobody, or antigen binding fragment thereof specifically binds CD4, CD44, CD47, epithelial cell adhesion molecule (EPCAM), carcinoembryonic antigen (CEA), cytotoxic T-lymphocyte-associated protein 4 (CTLA4), human epidermal growth factor receptor 2 (HER2), CD3, CD8, programmed cell death protein 1 (PD-1), lymphocyte activation gene 3 (LAG-3), or T cell immunoglobulin and mucin domain-containing protein 3 (TIM3).

In any of the above aspects, or embodiments thereof, the polynucleotide is an aptamer.

In any of the above aspects, or embodiments thereof, the polypeptide includes one or more unnatural amino acids or the polynucleotide includes one or more unnatural nucleobases.

In any of the above aspects, or embodiments thereof, the fusion polypeptide further includes a blocking domain that inhibits the polypeptide ligase activity. In any of the above aspects, or embodiments thereof, the blocking domain includes at least a fragment of a wild-type biotin protein ligase. In any of the above aspects, or embodiments thereof, the blocking domain includes a C-terminal domain of a biotin protein ligase. In any of the above aspects, or embodiments thereof, the C-terminal domain has at least 85% amino acid sequence identity to the following amino acid sequence: ENLYFQGSFKEFKGKIESKMLYLGEEVKLLGEGKITGKLVGLSEKGGALILTEEGIKE ILSGEFSLRRSGGS (SEQ ID NO: 3). In any of the above aspects, or embodiments thereof, the blocking domain is linked to the biotin protein ligase variant by a linker. In any of the above aspects, or embodiments thereof, the linker is a cleavable linker. In any of the above aspects, or embodiments thereof, the cleavable linker includes a protease recognition sequence. In any of the above aspects, or embodiments thereof, the protease recognition sequence is targeted by a furin, Tobacco Etch Virus (TEV), Rhinovirus 3C, Enterokinase, Factor Xa or other protease. In any of the above aspects, or embodiments thereof, the polypeptide further comprises a flexible spacer sequence. In any of the above aspects, or embodiments thereof, the flexible spacer sequence includes one or more of the following sequences: GGGS (SEQ ID NO: 4), GGGGG (SEQ ID NO: 5), GSGSGS (SEQ ID NO: 6), GGSGGS (SEQ ID NO: 7), GGGGS (SEQ ID NO: 8), GGGGSLVPRGSGGGGS (SEQ ID NO: 9), GGSGGHMGSGG (SEQ ID NO: 10), VEGGSGGSGGSGGSGGV (SEQ ID NO: 11), and GSTSGSGXPGSGEGSTKG (SEQ ID NO: 12). In any of the above aspects, or embodiments thereof, the flexible spacer sequence includes 2, 3, 4, or 5 repeats of the spacer sequence.

In any of the above aspects, or embodiments thereof, the fusion polypeptide further includes a Spycatcher domain or chaperone domain.

In any of the above aspects, or embodiments thereof, the targeting moiety targets the biotin protein ligase to the surface of a cell. In any of the above aspects, or embodiments thereof, the cell is an immune cell or a neoplastic cell.

In any of the above aspects, or embodiments thereof, the fusion polypeptide further includes a detectable moiety. In any of the above aspects, or embodiments thereof, the detectable moiety is: APC, PE, FITC, Alexa-fluor, AF488, Cy5, cyanines, or TAMRA.

In any of the above aspects, or embodiments thereof, the biotin protein ligase targets a chemotherapeutic agent bound to streptavidin or bound to an anti-biotin humanized antibody, to the surface of a cell decorated with biotin. In any of the above aspects, or embodiments thereof, the chemotherapeutic agent is auristatin, cisplatin, Docetaxel, Paclitaxel, carboplatin, Decitabine, azacitidine, 5-fluorouracil, gemcitabine, or methotrexate.

In any of the above aspects, or embodiments thereof, the fusion polypeptide is used to target a polynucleotide bound to a biotin binding moiety to the surface of a cell decorated with biotin. In any of the above aspects, or embodiments thereof, the polynucleotide is a DNA, RNA, antisense oligonucleotide, siRNA, or viral vector.

In any of the above aspects, or embodiments thereof, the biotin protein ligase is used to target a nanoparticle bound to biotin binding moiety to the surface of a cell decorated with biotin.

In any of the above aspects, or embodiments thereof, the biotin protein ligase is used to target a therapeutic polypeptide bound to biotin binding moiety to the surface of a cell decorated with biotin. In any of the above aspects, or embodiments thereof, the therapeutic polypeptide is a growth factor, cytokine, enzyme, transcription factor, Fc domain, monoclonal antibody, scFv or other antibody, antigen binding fragment, receptor agonist, or immunogen.

In any of the above aspects, or embodiments thereof, the biotin binding moiety is an anti-biotin antibody, avidin, streptavidin, or variant of any of the aforementioned biotin binding moieties.

In any of the above aspects, or embodiments thereof, the biotin protein ligase targets a cytotoxin bound to a biotin binding moiety to the surface of a cell decorated with biotin. In any of the above aspects, or embodiments thereof, the cytotoxin is ricin, diphtheria toxin, pseudomonas exotoxin (PE), ribosome-inactivating proteins (RIPs), botulinum toxin, disintegrin, melittin, chlorotoxin (CTX), colicins, actinoporin, haemolysins, aerolysins; cytolysin A, or cholesterol-dependent cytolysin.

In any of the above aspects, or embodiments thereof, the biotin protein ligase targets a Crispr bound to a biotin binding moiety to the surface of a cell decorated with biotin.

In any of the above aspects, or embodiments thereof, the biotin protein ligase targets an antibody-drug conjugate comprising an anti-biotin humanized antibody and an agent to the surface of a cell decorated with biotin. In any of the above aspects, or embodiments thereof, the agent is an auristatin, maytansanoid, benzodiazepine, Pseudomonas aeruginosa exotoxin PE38, calicheamicin, diphtheria toxin, irinotecan, duocarmycin, exatecan, Staphylococcus aureus enterotoxin A/E-120, doxorubicin, tubulysin, antibacterial antibiotic, shigatoxin, ricin, or urease.

In any of the above aspects, or embodiments thereof, the fusion polypeptide further includes a signal polypeptide.

In any of the above aspects, or embodiments thereof, the polynucleotide is RNA, DNA, or an RNA/DNA hybrid. In any of the above aspects, or embodiments thereof, the polynucleotide comprises an unnatural nucleobase.

In any of the above aspects, or embodiments thereof, the contacting is carried out in the presence of exogenous ATP. In any of the above aspects, or embodiments thereof, the contacting is carried out in the presence of an endogenous ATP. In any of the above aspects, or embodiments thereof, the contacting is carried out in the presence of exogenous biotin.

In any of the above aspects, or embodiments thereof, the cell is a neoplastic cell. In any of the above aspects, or embodiments thereof, the cell is a tumor cell, and wherein the tumor cell is within a tumor microenvironment (TME). In any of the above aspects, or embodiments thereof, the TME is characterized by the presence of an ATP concentration of from about 10 μM to about 1 mM. In any of the above aspects, or embodiments thereof, the TME is characterized by the presence of an ATP concentration of from about 50 μM to about 200 μM.

In any of the above aspects, or embodiments thereof, the agent is a polynucleotide, polypeptide, or small molecule.

In any of the above aspects, or embodiments thereof, the polypeptide specifically binds a polypeptide on the surface of a target cell.

In any of the above aspects, or embodiments thereof, the agent is a detectable moiety. In any of the above aspects, or embodiments thereof, the detectable moiety is a fluorophore. In any of the above aspects, or embodiments thereof, the detectable moiety is APC, PE, FITC, Alexa-fluor, Cy5, or TAMRA.

In any of the above aspects, or embodiments thereof, the agent is a chemotherapeutic agent. In any of the above aspects, or embodiments thereof, the chemotherapeutic agent is auristatin, cisplatin, Docetaxel, Paclitaxel, carboplatin, Decitabine, azacitidine, 5-fluorouracil, gemcitabine, or methotrexate.

In any of the above aspects, or embodiments thereof, the polynucleotide is an mRNA, siRNA, or antisense RNA. In any of the above aspects, or embodiments thereof, the polynucleotide is a viral vector.

In any of the above aspects, or embodiments thereof, the agent is a nanoparticle.

In any of the above aspects, or embodiments thereof, the polynucleotide encodes a therapeutic polypeptide. In any of the above aspects, or embodiments thereof, the therapeutic polypeptide is a growth factor, cytokine, enzyme, transcription factors, Fc domain, monoclonal antibody, scFv or other antibody fragment, receptor agonist, immunogen for use in a protein vaccine.

In any of the above aspects, or embodiments thereof, the biotin binding moiety is streptavidin, anti-biotin antibody, avidin, or avidin variants.

In any of the above aspects, or embodiments thereof, the agent is a cytotoxin. In any of the above aspects, or embodiments thereof, the cytotoxin is ricin, diphtheria toxin, pseudomonas exotoxin (PE), ribosome-inactivating proteins (RIPs), botulinum toxin, disintegrin, melittin, chlorotoxin (CTX), colicins, actinoporin, haemolysins, aerolysins; cytolysin A, or cholesterol-dependent cytolysin.

In any of the above aspects, or embodiments thereof, the agent is a Crispr

In any of the above aspects, or embodiments thereof, the fusion polypeptide further includes a signal polypeptide or chaperone domain.

In any of the above aspects, or embodiments thereof, the biotin binding moiety is an anti-biotin antibody, or biotin-binding fragment thereof.

In any of the above aspects, or embodiments thereof, the agent is an auristatin, maytansanoid, benzodiazepine, Pseudomonas aeruginosa exotoxin PE38, calicheamicin, diphtheria toxin, irinotecan, duocarmycin, exatecan, Staphylococcus aureus enterotoxin A/E-120, doxorubicin, tubulysin, antibacterial antibiotic, shigatoxin, ricin, or urease.

In any of the above aspects, or embodiments thereof, the biotin protein ligase is divided into one or more fragments, each of which is independently delivered to the cell.

In any of the above aspects, or embodiments thereof, the fusion protein further includes an inhibitory C-terminal of biotin protein ligase.

In any of the above aspects, or embodiments thereof, the system further includes a protease capable of cleaving the inhibitory C-terminal of biotin protein ligase.

The invention provides an enzymatic system for precise cell targeting. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound. In some embodiments, the agent is a fusion polypeptide comprising a biotin protein ligase as disclosed herein. In an embodiment, the agent is an antibody drug conjugate comprising an antibody, or antigen binding fragment thereof, which specifically binds biotin. In some embodiments, the agent is a therapeutic compound, such as a chemotherapeutic compound, a cytotoxic compound, for conjugation to an antibody which specifically binds biotin.

By “ameliorate” is meant decrease, suppress, attenuate, diminish, arrest, or stabilize the development or progression of a disease.

The term “adaptor” refers a sequence that is added, for example by ligation, to a nucleic acid. The length of an adaptor may be from about 5 to about 100 bases, and may provide a sequencing primer binding site (e.g., an amplification primer binding site), and a molecular barcode such as a sample identifier sequence or molecule identifier sequence, preferably a unique identifier sequence. An adaptor may be added to 1) the 5′ end, 2) the 3′ end, or 3) both ends of a nucleic acid molecule. Double-stranded adaptors contain a double-stranded end ligated to a nucleic acid. An adaptor can have an overhang or may be blunt ended. As will be described in greater detail below, a double stranded adaptor can be added to a fragment by ligating only one strand of the adaptor to the fragment. The sequence of the non-ligated strand of the adaptor may be added to the fragment using a polymerase. Y-adaptors and loop adaptors are type of double-stranded adaptors.

By “alteration” is meant a change (increase or decrease) in the expression levels, structure, or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” (Ab) refers to an immunoglobulin molecule that specifically binds to, or is immunologically reactive with, a particular antigen, and antigen binding fragments thereof. Exemplary antibodies encompass polyclonal, monoclonal, genetically and molecularly engineered and otherwise modified forms of antibodies, including, but not limited to, chimeric antibodies, humanized antibodies, heteroconjugate antibodies (e.g., bi-tri- and quad-specific antibodies, diabodies, triabodies, and tetrabodies), and antigen-binding fragments of antibodies, including e.g., Fab′, F(ab′)₂, Fab, Fv, rlgG, and scFv fragments. Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)₂ fragments, that are capable of specifically binding to a target protein. Antibodies may include chimeric antibodies; recombinant and engineered antibodies, and antigen binding fragments thereof.

By “antibody drug conjugate” is meant a compound including an antibody, or antigen binding fragment thereof, covalently attached to a drug or agent via a chemical linker. In some embodiments, the antibody is an anti-biotin humanized antibody, or biotin binding fragment thereof. In some embodiments, the agent or drug conjugated to an antibody in an antibody-drug conjugate is an auristatin, maytansanoid, benzodiazepine, Pseudomonas aeruginosa exotoxin PE38, calicheamicin, diphtheria toxin, irinotecan, duocarmycin, exatecan, Staphylococcus aureus enterotoxin A/E-120, doxorubicin, tubulysin, antibacterial antibiotic, shigatoxin, ricin, or urease

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art. In some embodiments, antigen-binding fragments (e.g., Fab′, F(ab′)₂, Fab, scFab, Fv, rlgG, and scFv fragments) of an antibody are provided.

Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)₂, a fragment of an antibody molecule containing a monovalent antigen-binding portion of an antibody molecule, obtained by treating an intact antibody with the enzyme pepsin (i.e., a dimer of Fab′ fragments held together by two disulfide bonds).

As used herein, the term “antisense oligonucleotide” refers to a polynucleotide that is substantially or 100% complementary to a target nucleic acid of interest, and that reduces or eliminates protein expression. For example, an antisense strand may be complementary, in whole or in part, to a molecule of mRNA (messenger RNA), an RNA sequence that is not mRNA (e.g., microRNA, piwiRNA, tRNA, rRNA and hnRNA) or a sequence of DNA that is either coding or non-coding. The terms “antisense strand” and “guide strand” are used interchangeably herein.

By “aptamer” is meant a single-stranded polynucleotide that specifically binds to a protein.

By “biotin” is meant an organic hetero bicyclic compound having the following structure:

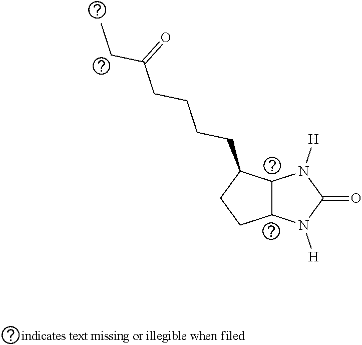 [SMILES: CCC(=O)CCCC[C@@H]1CCC2NC(=O)NC21]

By “biotin protein ligase” or “biotin ligase” or “biotin polypeptide ligase” is meant an enzyme that ligates biotin to a target site, that comprises a biotin protein ligase active site or variant thereof, and/or that has at least about 85% amino acid sequence identity to Ensembl: AAC06798 or a functional fragment thereof. In one embodiment, a biotin protein ligase has at least about 85% amino acid sequence identity to the following exemplary amino acid sequence: MFKNLIWLKEVDSTQERLKEWNVSYGTALVADRQTKGRGRLGRKWLSQEGGLYF SFLLNPKEFENLLQLPLVLGLSVSEALEEITEIPFSLKWPNDVYFQEKKVSGVLCELSK DKLIVGIGINVNQREIPEEIKDRATTLYEITGKDWDRKEVLLKVLKRISENLKKFKEKS FKEFKGKIESKMLYLGEEVKLLGEGKITGKLVGLSEKGGALILTEEGIKEILSGEFSLR RS (SEQ ID NO: 13), or a functional fragment thereof. In one embodiment, a biotin protein ligase comprises the following active site: GRGRXGRKW (SEQ ID NO: 14), wherein the X is L or P. In another embodiment, the biotin protein ligase comprises a truncation at the C terminus.

By “biotin protein ligase polynucleotide” is meant a protein encoding a biotin protein ligase. In one embodiment, a biotin protein ligase attaches biotin to an amino acid on the cell surface. In another embodiment, a biotin protein ligase attaches biotin to any solvent residue exposed amino acid. In another embodiment, the biotin ligase attaches biotin to a lysine residue at the target site or to a nearby lysine residue (e.g., 5 nm, 10 nm, 20 nm, or more away) or other amino group (e.g., non-natural amino acid or exposed N-terminus). Biotin protein ligases are described, for example, in the following references: Chapman-Smith et al., Biomol. Eng. 1999; 16:119-125. doi: 10.1016/S1050-3862 (99) 00046-7, Bagautdinov et al., J. Biol. Chem. 2008; 283:14739-14750. doi: 10.1074/jbc.M709116200, and Paparella et al., Curr. Top. Med. Chem. 2014; 14:4-20, each of which are incorporated herein by reference in their entirety.

By “Chimeric Antigen Receptor” or alternatively a “CAR” is meant a polypeptide capable of providing an immune effector cell with specificity for a target cell. In embodiments, the target cell is a cancer cell. In some embodiments, a CAR comprises at least an extracellular antigen binding domain, a transmembrane domain and a cytoplasmic signaling domain comprising a functional signaling domain derived from a stimulatory molecule and/or costimulatory molecule. In embodiments, the stimulatory molecule is the zeta chain associated with the T cell receptor complex. In one embodiment, the cytoplasmic signaling domain further comprises one or more functional signaling domains derived from at least one costimulatory molecule. In one embodiment, the CAR comprises a chimeric fusion protein comprising an extracellular antigen binding domain, a transmembrane domain and an intracellular signaling domain comprising a functional signaling domain derived from a stimulatory molecule. In one embodiment, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one embodiment, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one embodiment, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one embodiment the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

By “chemotherapeutic agent” is meant an agent that inhibits cancer cell proliferation, inhibits cancer cell survival, increases cancer cell death, inhibits and/or stabilizes tumor growth, or that is otherwise useful in the treatment of cancer. In embodiments, chemotherapeutic agents provided herein are used as part of an immunotherapy. In embodiments, chemotherapeutic agents provided herein contain an immune checkpoint blockade (ICB). In embodiments, the ICB contains a PD-1/PD-L1 checkpoint inhibitor (e.g., atezolizumab, avelumab, BMS-936559, MDX-1105, cemiplimab, durvalumab, nivolumab, and/or pembrolizumab). In embodiments, an PD-1/PD-L1 checkpoint inhibitor contains an anti-CTLA-4 and/or anti-PD-1 antibody. In embodiments, the chemotherapeutic agents provided herein contain a CAR-T that has been modified to reduce or eliminate expression or activity of an NKG2A and/or CD94 polypeptide.

One of skill in the art can readily identify a chemotherapeutic agent of use in a method for treating a cancer described herein (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In some embodiments of any of the aspects, the combination of agents provided herein decrease cancer cell proliferation or survival by at least 10%, 20%, 30%, 40%, 50%, 60%, 70%, 80%, 90%, 95%, or 100%, and includes inducing cell death (apoptosis) in a cell or cells within a cell mass.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “complementary” is meant capable of pairing to form a double-stranded nucleic acid molecule or portion thereof. In one embodiment, an antisense molecule is in large part complementary to a target sequence. The complementarity need not be perfect, but may include mismatches at 1, 2, 3, or more nucleotides.

By “corresponds” is meant comprising at least a fragment of a double-stranded gene, such that a strand of the double-stranded inhibitory nucleic acid molecule is capable of binding to a complementary strand of the gene.

By “decreases” is meant a reduction by at least about 5% relative to a reference level. A decrease may be by 5%, 10%, 15%, 20%, 25% or 50%, or even by as much as 75%, 85%, 95% or more and any intervening percentages

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In some embodiments, the disease is a neoplasia. In other embodiments, the disease in an autoimmune disorder.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “extein” is meant a protein fragment fused to an intein or split intein. In embodiments, an extein is a protein fragment fused to an N-intein or a C-intein.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “fusion protein” as used herein refers to a hybrid polypeptide which comprises protein domains from at least two different proteins.

By “heterologous,” or “exogenous” is meant a polynucleotide or polypeptide that 1) has been experimentally incorporated into a polynucleotide or polypeptide sequence in which the polynucleotide or polypeptide is not normally found in nature; or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

A “host cell” or “cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “intein” is meant a protein segment capable of exercising itself and concurrently ligating flanking exteins in a process known as protein splicing. The process of an intein excising itself and joining the remaining portions of the protein is herein termed “protein splicing” or “intein-mediated protein splicing.” In some embodiments, an intein is a trans-splicing intein (also referred to as a “split intein”). In the case of trans-splicing inteins, a full-length polypeptide is split into two separate fragments and the C-terminus of the N-terminal fragment is fused to an N-terminal fragment of a split intein intein (N-intein) and the N-terminus of the remaining C-terminal fragment is fused a C-terminal fragment of a split intein (C-intein).

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein, polynucleotide, lipid, or other moiety having an alteration in expression level or activity that is associated with a disease or disorder. In one embodiment, the marker is presented on an external surface of a cell.

A “nanobody” as used herein also is used synonymously to refer to a single-domain antibody. A nanobody refers to an antibody fragment or portion which contains a single monomeric variable domain (V_{H}) naturally occurring in the Camelidae family or synthetically derived from the heavy chain of an antibody. Such single-domain binding molecules combine high antigen affinity in the absence of complement-dependent or cell-mediated cytotoxicity due to the lack of a constant (Fc) region in these molecules.

By “operably linked” refers to a functional linkage between a regulatory sequence and a coding sequence, where a first polynucleotide is positioned adjacent to a second polynucleotide that directs transcription of the first polynucleotide when appropriate molecules (e.g., transcriptional activator proteins) are bound to the second polynucleotide. The described components are therefore in a relationship permitting them to function in their intended manner. For example, placing a coding sequence under regulatory control of a promoter means positioning the coding sequence such that the expression of the coding sequence is controlled by the promoter.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide.

By “positioned for expression” is meant that the polynucleotide of the disclosure (e.g., a DNA molecule) is positioned adjacent to a DNA sequence that directs transcription and translation of the sequence (i.e., facilitates the production of, for example, a recombinant microRNA molecule described herein).

The term “promoter” as used herein refers to a sequence of DNA that directs the expression (transcription) of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules of some aspects and embodiments herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively, or in addition, mutations are made to CDR amino acid residues to optimize antigen binding using art recognized techniques. scFv fragments are described, for example, in WO 2011/084714; incorporated herein by reference.

According to some aspects and embodiments herein, antibody fragments are understood as meaning functional parts of antibodies, such as Fc, Fab, Fab′, Fv, F(ab′)2, scFv. According to some aspects and embodiments herein, corresponding biological active fragments are to be understood as meaning those parts of antibodies which are capable of binding to an antigen, such as Fab, Fab′, Fv, F(ab′)2, and scFv.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

A “selectable marker” that is suitable for use in the identification and selection of cells transformed or transfected with a cloning vector. Marker genes include genes that provide tetracycline resistance or ampicillin resistance, for example. Non-limiting examples of a selectable marker or detectable moiety include a fluorophore, an antibody resistance cassette, a capture molecule, a biotin molecule, streptavidin molecule, or an antigen.

By “selectable moiety gene” is meant a gene or nucleic acid sequence that is attached to a sequence of a gene of interest for identification and/or quantification. Non-limiting examples, of molecules encoded by a “selectable moiety gene” include a fluorophore, green fluorescent protein, enhanced green fluorescent protein, an antibody resistance cassette, an antigen, a capture molecule, a biotin molecule, a streptavidin molecule, or another selectable or identifiable molecule.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 mg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “target cell” is meant a cell acted upon by a biotin protein ligase or other effector molecule.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “tumor microenvironment” or “TME” is meant the environment around a tumor, including the surrounding blood vessels, immune cells, fibroblasts, signaling molecules, and the extracellular matrix. In some embodiments, the TME is characterized by the presence of an increased ATP concentration relative to the extracellular environment of healthy tissue. In some embodiments, the TME is characterized by the presence of an ATP concentration of from about 10 μM to about 1 mM, from about 20 μM to about 900 μM, from about 30 μM to about 800 μM, from about 40 μM to about 700 μM, from about 50 μM to about 600 μM, from about 50 μM to about 400 μM, from about 50 μM to about 200 μM, at least 10 μM, at least 15 μM, at least 20 μM, at least 25 μM, at least 30 μM, at least 35 μM, at least 40 μM, at least 45 μM, or at least 50 μM.

Unless specifically stated or obvious from context, as used herein, the term “or” is understood to be inclusive. Unless specifically stated or obvious from context, as used herein, the terms “a”, “an”, and “the” are understood to be singular or plural.

By “vector” is meant a nucleic acid molecule, for example, a plasmid, cosmid, virus, or bacteriophage that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a schematic diagram showing a target cell having receptors on the surface. An enzyme may be used, to conjugate the triangular labels to the surface of the target cell.

FIG. 2 is a general reaction scheme of biotin ligase.

FIG. 3 provides an alignment of the A. aeolicus and E. coli biotin ligase sequences including the active site. The arrow indicates the arginine that is modified to render the ligases non-specific. Conserved amino acids are indicated in black boxes. Figure discloses SEQ ID NOS 43-45, respectively, in order of appearance.

FIGS. 4A-4F describe experiments using E. coli BirA. FIG. 4A is a reaction scheme for E. coli BirA. FIG. 4B provides the structure of BirA with an exemplary nanobody useful as a targeting domain. FIGS. 4C-4F are images of Western blots stained with an anti-His tag antibody (linked to HRP and detected via chemiluminescence). Figure discloses SEQ ID NO: 15. FIG. 4C is an image showing that R118G BirA was expressed in E. coli as a fusion protein with a chaperone (mRID). Shown are the cell lysis pellet (P) and supernatant(S) fractions for the indicated range of inducer concentrations (μM). FIG. 4D is an image of a Western blot showing that BirA and an anti-CD3 nanobody-BirA fusion were expressed in E. coli using NusA as a fusion partner; shown are pellet and supernatant fractions. FIG. 4E is an image of a blot showing protein expression in supernatant (M), pellet (P) and lysis supernatant(S) fractions of cultured HEK93T cells expressing for a multi-mutant BirA variants and several nanobody-mutant BirA fusions. FIG. 4F is an image of a blot showing protein expression in culture supernatant and lysis fractions for nanobody-multi-mutant BirA fusions expressed with four different signal peptides in HEK293T cells.

FIG. 5 shows that MicroID is a truncation of aaBL/BioID2 after K171, cleaving off the C-terminal domain. UltraID includes an additional active site mutation, L41P. The amino acid sequences for aaBL/BioID2, MicroID, and UltraID are listed. Figure discloses SEQ ID NOS 17, 19, and 1, respectively, in order of appearance.

FIGS. 6A-6E show expression and activity testing of aaBL constructs. FIG. 6A, provides the structure of A. aeolicus biotin ligase. FIG. 6B is an image of a Western blot showing protein expression in Pellet (P) and supernatant(S) fractions for aaBL expressed in E. coli across the indicated range of inducer concentrations (μM) and the indicated expression time. FIG. 6C is an image of a Western blot showing protein expression of Chaperone-aaBL and aCTLA4 nanobody-aaBL fusions. The proteins were expressed and purified successfully. FIG. 6D shows that a chaperone-aaBL fusion (1 μM) was incubated with 500 μM biotin and 5 mM ATP 37° C. for 24 hours, then the blot was stained with streptavidin. FIG. 6E Same as FIG. 6D), for aCTLA4 nanobody-aaBL fusion.

FIG. 7 shows the labeling scheme for UltraID (A. aeolicus biotin ligase variant) of biotin onto a target cell using ATP. The biotin ligase variant reacts with any exposed lysine residue, covalently attaches biotin, has a structure and activity that are well characterized, has high activity, and uses endogenous substrates that are present or readily supplied (i.e., biotin and ATP).

FIGS. 8A-8C are FACS analyses. FIG. 8A B3Z cells were treated with 10 μM enzyme construct, 500 μM biotin and 5 mM ATP for 24 hours, then washed, stained with streptavidin-APC, and analyzed via flow cytometry. Shown are the % SAv+, gated on single cells. Also included is a positive control labeled with NHS-biotin. FIG. 8B Histograms representing data from FIG. 8A as well as a two-hour enzyme incubation period with the same conditions. FIG. 8C B3Z cells were stained with PE-aCTLA-4 monolonal antibody (or incubated in staining buffer alone) for one hour and analyzed via flow cytometry.

FIG. 9 shows results of flow cytometry analysis. Treatment of each cell type was conducted with 10 nM each ultraID construct for 1 hour at 37° C. with 5 mM ATP and 50 μM biotin, then staining with streptavidin-APC to assess target-specific biotin labeling. The SpyCatcher-only construct represents untargeted labeling, the aCTLA-4 nanobody represents off-target labeling, and the aCD4 nanobody represents on-target labeling for U937s.

FIG. 10 are bar graphs showing that biotin protein ligase conjugated to an anti-human CD4 nanobody specifically biotinylated the CD4-expressing human cell line. All other targeting ligand/cell line combinations exhibited minimal biotin labeling.

FIG. 11 is a graph. U-937 cells (CD4⁺ human monocyte-derived line) were treated with the indicated concentration of either non-targeted (SpyCatcher-ultraID) or on-target (aCD4-ultraID) enzyme construct for 1 hour at 37° C. with 1 mM ATP and 50 μM biotin, then stained with streptavidin-APC and analyzed via flow cytometry. aCD4-targeted enzyme exhibited a dose-dependent efficiency with robust activity at treatment concentrations as low as 0.5 nM and clear distinction from the non-targeted enzyme up to ˜100 nM. Combination of three experimental replicates (different days, different batches of enzyme). Data was fitted as non-linear [agonist] v. response (three parameters) with Prism software.

FIG. 12 is a schematic that showing that differences in the tissue micro-environment can be exploited to increase specificity of biotin protein ligase activity (effectively introducing a second marker).

FIG. 13 shows results of flow cytometry analysis. The analysis shows that B16 cells are CD47+ and can serve as an initial test case for tumor cell targeting.

FIG. 14 shows results of flow cytometry analysis. The analysis shows that B16 cells were treated with the indicated concentration of aCD47-ultraID, 50 μM biotin, and 1 mM ATP for 1 h at 37° C. then stained and analyzed via flow cytometry. aCD47-ultraID efficiently targeted b16 cells with near-complete labeling even at 1 nM.

FIG. 15 is a bar graph showing that targeted-ultraID exhibited a 10-fold increase in biotin labeling over the non-targeted ultraID at 1 nM treatment, with targeted activity increasing further at 10 and 100 nM.

FIG. 16 shows results of flow cytometry analysis. The analysis shows that stained U-937s with anti mCD47-AF488 antibody for 1 h at 4° C. U-937s do not appear to be stained by an anti mCD47 antibody and can serve as a negative control for aCD47-ultraID targeting.

FIGS. 17A-17B provide tumor cell targeting results. FIG. 17A are flow cytometry analysis results showing that U-937s (which do not express mCD47) were treated with 10 nM of the indicated ultraID construct and analyzed. Note that aCD4-ultraID represents a positive control as U-937s are hCD4+. aCD47-ultraID did not biotinylate U-937s, confirming that its activity is specific to mCD47+ cells and represents successful targeting based on a tumor marker. FIG. 17B are graphs showing tumor cell targeting across a range of enzyme concentrations. B16 cells were treated with aCD47-ultraID at the indicated concentration, 50 μM biotin and 1 mM ATP for 1 h at 37° C., then stained with streptavidin-APC and analyzed via flow cytometry.

FIGS. 18A-18B are graphs and bar graphs. FIG. 18A shows that B16 cells were treated with 25 nM of either non-targeted or aCD47-ultraID, 50 μM biotin and the indicated concentration of ATP for 1 hour at 37° C. then analyzed via flow cytometry. aCD47-ultraID is active at ATP concentrations typically found in the TME (e.g., 50-200 μM (Vultaggio-Poma, V. et al. Cells 9, 2496 (2020), doi: 10.3390/cells9112496)). FIG. 18B shows tumor cell targeting across a tumor-relevant ATP concentration range at several time points. B16 cells were treated with 25 nM aCD47-ultraID, 50 μM biotin and the indicated concentration of ATP for 1, 3, or 24 hours at 37° C., then stained with streptavidin-APC and analyzed via flow cytometry.

FIG. 19 is a graph. U-937 cells were treated with non-targeted or CD4-targeted enzyme (10 nM, with 50 μM biotin and 1 mM ATP) for 1 h at 37° C. BioID2 was substantially less active than ultraID. Notably, no difference between targeted and non-targeted BioID2 was observed under these conditions.

FIG. 20 is a schematic showing the design of a proteolytically-activated ultraID, using a hindered variant comprised of the ultraID sequence with the C-terminal domain from BioID2 reincorporated and connected via a cleavable linker. The amino acid sequences for BioID2, ultraID, and ultraID-TEV-Cterm are listed. TEV denotes tobacco etch virus. Figure discloses SEQ ID NOS 46, 1, and 47, respectively, in order of appearance.

FIG. 21 shows a stained gel with 1) ultraID-TEV site-Cterm and 2) ultraID-TEV site-Cterm+TEV protease. ultraID-TEV-Cterm was treated with TEV protease (NEB) overnight at 4° C. then analyzed by SDS-PAGE. A small amount of cleaved ultraID is detectable after TEV treatment, but cleavage appears to be relatively inefficient.

FIG. 22 is a schematic showing that incorporating a spacer improves proteolytic cleavage. Incorporating a flexible GGGS spacer (SEQ ID NO: 4) after the TEV site introduces greater flexibility and allows the TEV protease greater access, improving cleavage efficiency. The amino acid sequences for ultraID-TEV-Cterm and ultraID-TEV-GGGS-Cterm are listed. Figure discloses SEQ ID NOS 47-48, respectively, in order of appearance.

FIG. 23 is a gel showing in lanes 1) ultraID-TEV site-Cterm; 2) ultraID-TEV site-Cterm+TEV protease; 3) ultraID-TEV site-GGGS-Cterm; and 4) ultraID-TEV site-GGGS-Cterm+TEV protease. Hindered ultraID constructs were treated with TEV protease overnight at 4° C. then analyzed by SDS-PAGE. The addition of a GGGS spacer (SEQ ID NO: 4) yielded more efficient proteolytic cleavage, but the hindered ultraID remained predominantly intact.

FIG. 24 includes two graphs showing that after pre-treating hindered ultraID variants with TEV protease (NEB) for 24 hours, the pre-cleaved constructs were conjugated to an aCD4 nanobody and used to treat U-937s. Some gain in biotin-labeling activity was observed with TEV treatment, indicating some release of the more active ultraID. Figure discloses SEQ ID NOS 4 and 4, respectively.

FIG. 25 is a gel showing in lanes 1) ultraID-TEV site-Cterm; 2) ultraID-TEV site-Cterm+TEV protease; 3) ultraID-TEV site-GGGS-Cterm; 4) ultraID-TEV site-GGGS-Cterm+TEV protease; 5) ultraID-TEV site-(GGGS) 2-Cterm; 6) ultraID-TEV site-(GGGS) 2-Cterm+TEV protease; 7) ultraID-TEV site-(GGGS)₃-Cterm; and 8) ultraID-TEV site-(GGGS)₃-Cterm+TEV protease. Hindered ultraID constructs were treated with TEV protease (NEB) overnight at 4° C. then analyzed by SDS-PAGE. TEV cleavage efficiency increased with the addition of the (GGGS)ₙ spacer (SEQ ID NO: 4), with the most significant improvement observed with (GGGS)₃ (SEQ ID NO: 15).

FIGS. 26A-26B are a gel and bar graph. FIG. 26A is a gel showing in lanes 1) ultraID-TEV site-Cterm; 2) ultraID-TEV site-Cterm+TEV protease; 3) ultraID-TEV site-GGGS-Cterm; 4) ultraID-TEV site-GGGS-Cterm+TEV protease; 5) ultraID-TEV site-(GGGS) 2-Cterm; 6) ultraID-TEV site-(GGGS) 2-Cterm+TEV protease; 7) ultraID-TEV site-(GGGS)₃-Cterm; and 8) ultraID-TEV site-(GGGS)₃-Cterm+TEV protease. Hindered ultraID constructs were treated with our recombinantly expressed SpyCatcher-TEV protease overnight at 4° C. then analyzed by SDS-PAGE. SpyCatcher-TEV was active, with the most efficient cleavage observed for the (GGGS)₃ linker (SEQ ID NO: 15) variant. FIG. 26B shows that U-937 cells were treated with 10 nM of the indicated aCD4-biotin ligase and 100 nM SpyCatcher-TEV protease, 50 μM biotin and 1 mM ATP for 1 h at 37° C., then stained with streptavidin-APC. An increase in activity is observed with TEV treatment with some proteolytic cleavage appearing on this timescale using a non-targeted TEV protease. Figure discloses SEQ ID NOS 4, 49, 15, 4, 49, and 15, respectively, in order of appearance.

FIG. 27 includes two graphs showing that U-937 cells were treated with 0, 1, or 10 nM of either non-targeted or aCD4-ultraID, 50 μM biotin and the indicated concentration of ATP for 1 hour at 37° C. then analyzed via flow cytometry. aCD4-ultraID remains active down to ˜50 μM ATP, although the activity drops substantially. Comparing activity across longer timescales will likely be more representative of an in vivo setting. The targeted enzyme was active at typical tumor micro-environment extracellular ATP concentrations.

FIG. 28 includes four graphs showing increased cell targeting after TEV treatment. Figure discloses “GGGS” as SEQ ID NO: 4.

FIGS. 29A-29C include three graphs showing that the enzyme-based targeting system selectively labels CD4+ cells in the presence of CD4″ decoy cells. FIG. 29A shows that allophycocyanin (APC) CD4 antibody efficiently distinguishes CD4+ target (U937) and CD4-decoy (Jurkat) cells in a mixed population of the two cell types. FIG. 29B shows results after target and decoy cells were mixed in defined ratios (given as % target cell within total population; all mixtures had 1e6 total cells/mL) then treated with either non-targeted biotin ligase or biotin ligase conjugated to an anti-CD4 (aCD4) nanobody (nAb) (25 nM enzyme, 50 μM biotin and 1 mM ATP), treated for 1 h at 37° C. then analyzed via flow cytometry, using streptavidin-phycoerythrin (PE SAv) to evaluate biotin labeling and APC CD4 Ab to distinguish target vs. decoy cells. Across all target: decoy ratios, only target cells were labeled with aCD4 biotin ligase. FIG. 29C shows the relative PE SAv signal for sub-populations from FIG. 29B and indicated that biotin labeling efficiency dropped slightly at low percentages of target cells but was relatively consistent (again with no detectable labeling of decoy population). Mean fluorescent intensity (MFI) is displayed in these graphs.

FIGS. 30A-30B include a graph and a table showing that enzyme-based targeting increases sensitivity over aCD47 nAb binding. FIG. 30A shows a comparison of the sensitivity of enzyme-based targeting to ‘conventional’ labeling (i.e., direct nAb binding). nAb-ultraID constructs were labeled directly with 1-10 molecules of biotin-NHS so that streptavidin staining could be used as a readout. B16 cells were treated with either 25 nM of ‘conventional’ biotinylated aCD47 nanobody construct or aCD47 nAb-linked biotin ligase (with 50 μM biotin and 1 mM ATP) for the indicated duration, then stained and analyzed via flow cytometry. In FIG. 30A the term “MFI” refers to Mean Fluorescent Intensity. FIG. 30B shows fold-change improvements for enzyme-based targeting relative to nanobody binding alone (normalized to molar equiv. of nAb for different biotinylation ratios).

FIGS. 31A-31E include ten graphs showing the results from determining optimal treatment conditions for targeted biotin ligase (enzyme). U937 and B16 cell lines were used as model systems to evaluate optimal treatment parameters, using CD4 and CD47-targeting nanobodies, respectively: FIG. 31A shows biotin ligase concentration, FIG. 31B shows ATP concentration, with the tumor-relevant concentration range indicated by the shaded region; FIG. 31C shows biotin concentration; FIG. 31D shows treatment time, in which cells were continuously treated for the indicated duration; FIG. 31E shows temperature comparison for 37° C. vs. room temperature treatment, evaluated at several shorter time points up to one hour. For all experiments shown in FIGS. 31A-31E, the following conditions were used unless otherwise indicated: 25 nM biotin ligase, 50 μM biotin, 1 mM ATP with a 1-hour treatment at 37° C. Cells were stained with streptavidin PE and analyzed via flow cytometry. Note that in FIGS. 31A-31C the x-axes are shown on a log scale while FIGS. 31D-31E have linear x-axes.

FIGS. 32A-32B include a schematic and two graphs showing that enzymatically-attached biotin is retained at the cell surface for several hours. Cells were treated with 25 nM biotin ligase, 50 μM biotin and 1 mM ATP; after one hour, treatment media was exchanged for fresh media (no enzyme, biotin or ATP) and incubated for the remainder of the experiment. FIG. 32A shows a schematic of the experimental design. Setup was staggered so that the staining and analysis were performed together. FIG. 32B shows the results. The experiment in FIGS. 32A-32B was performed in U937 cells (aCD4 nAb-biotin ligase) and B16 cells (aCD47 nAb-biotin ligase). Dotted vertical lines indicate end of 1 h biotin ligase treatment period, at which point treatment media was exchanged for fresh media (without enzyme, biotin or ATP).

FIGS. 33A-33B include a schematic and seven graphs showing that commercial antibodies can target biotin ligase to a range of cell types. FIG. 33A shows that click chemistry enables straightforward conjugation of commercially available antibodies to biotin ligase by attaching the pictured SpyTag peptide. SpyTag rapidly binds to SpyCatcher-biotin ligase fusion protein. Note that ‘X’ in the peptide sequence denotes the non-natural amino acid azidoornithine. Figure discloses SEQ ID NO: 50. FIG. 33B shows the results after a range of Abs were conjugated to the biotin ligase ultraID and used to target human cell lines: U937 (CD4, CD44) and MCF-7 (Her2); primary murine CD8 OT-I T cells (CD3, CD8a), and the murine B16 melanoma cell line (PD-L1, CD44). For all constructs, cells were treated with 10 nM biotin ligase (except for PD-L1, 25 nM), 50 μM biotin and 1 mM ATP for 1-2 h at 37° C. before staining with SAv PE and analyzing via flow cytometry. In each graph, the left peak represents non-targeted biotin ligase, and the right peak represents targeted (Ab) biotin ligase.

FIGS. 34A-34B include five graphs showing the results from using the enzyme-based targeting system in detecting exhaustion markers in tumor-infiltrating lymphocytes (TILs). FIG. 34A shows results after CD8 cells were isolated from B16 tumors and draining lymph nodes, then treated with targeted biotin ligase (10 nM, with biotin and ATP) for 1 h at 37° C., stained and analyzed via flow cytometry to evaluate targeted biotin labeling. For CD8a, the peaks are as follows, from left to right: unstained TILs; unstained T-cells from draining lymph nodes (dLNTs); targeted dLNTs; and targeted TILs. For TIM3, the peaks are as follows, from left to right: unstained TILs; unstained dLNTs; targeted dLNTs; and targeted TILs. For LAG-3, the peaks are as follows, from left to right: unstained TILs and unstained dLNTs (overlapping); targeted dLNTs; and targeted TILs. FIG. 34B shows the results from the same conditions as FIG. 34A but with B16 CD8 TILs as well as TILs from MC-38 tumors, showing separation into distinct LAG3hi and LAG31° populations detected with targeted biotin ligase.

### DETAILED DESCRIPTION OF THE INVENTION

The present disclosure provides compositions and methods for enzyme-mediated precise cell targeting.

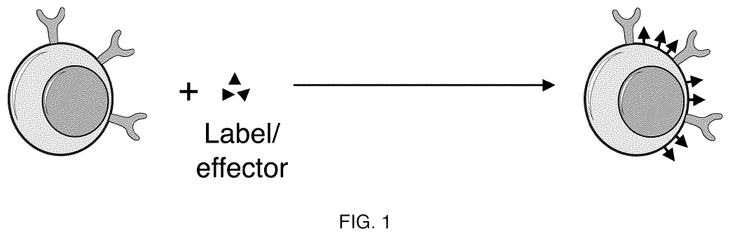

The invention is based, at least in part, on the discovery of fusion proteins comprising a biotin protein ligase and a targeting molecule (e.g., antibody, antigen-binding fragment thereof, nanobody) that may be used to conjugate biotin to cells of interest (FIG. 1, where biotin is shown as a black triangle). An agent (e.g., polypeptide (e.g., therapeutic polypeptide, cytokine), detectable moiety (fluorophore), polynucleotide (siRNA, antisense oligonucleotide) small molecule, toxin, chemotherapeutic) conjugated to a biotin binding moiety (e.g., anti-biotin antibody, streptavidin, avidin, etc.) is then delivered to the biotin decorated cells.

Advantageously, the ligase's catalytic nature provides signal amplification to improve sensitivity over conventional targeting methods. In some embodiments, activatable enzymatic versions are used to take advantage of local environment-specific features or incorporate a two-marker requirement for greater selectivity. In some embodiments, specificity is afforded by delivering the enzyme (e.g. ligase) in two fragments. Distinct sub-populations of immune and tumor cells are targeted for a range of applications (e.g., detection and tracking of distinct cell sub-populations, selective ablation of distinct cell sub-populations, delivering nucleic acids for gene therapy of distinct cell sub-populations).

### Biotin Ligases

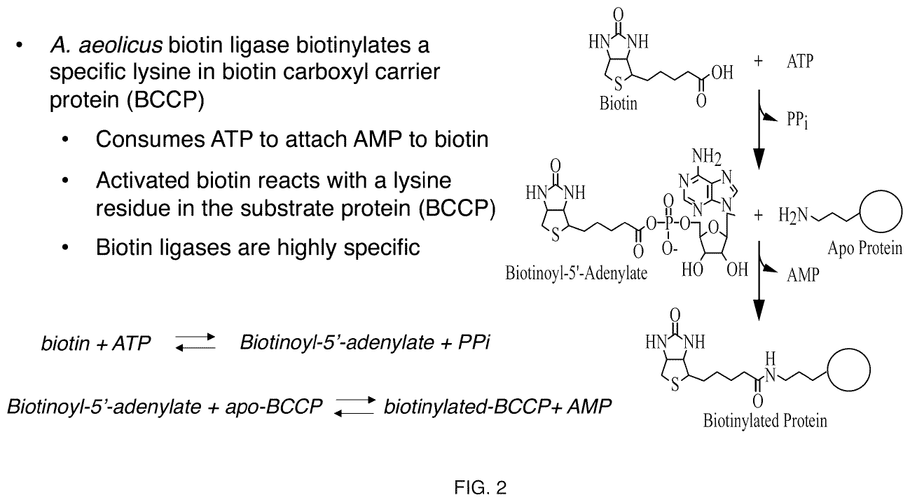

Biotin (vitamin H/vitamin B7), an essential coenzyme synthesized by plants and most prokaryotes, is required by all organisms. In cells, biotin in its physiologically active form is covalently attached at the active site of a class of important metabolic enzymes, the biotin carboxylase and decarboxylases. Biotin protein ligase (BPL), also known as holocarboxylase synthetase (EC 6.3.4.15), is the enzyme responsible for the covalent attachment of biotin to cognate proteins. Biotin is attached post-translationally by BPL via an amide linkage to a specific lysine residue of newly synthesized carboxylases in a two-step reaction. In E. Coli, biotin ligase alters gene expression by biotinylating the lysine residue of a specific target sequence on histones in the E. coli genome, serving as a negative regulator of the biotin biosynthesis operon. The general reaction scheme is shown in FIG. 2A single active site mutation, R118G, eliminates specificity for the substrate target sequence and instead causes the activated biotinyl-AMP species to be released, upon which it reacts with the primary amine of any proximal lysine residues. This variant is capable of biotinylating any lysines within a ˜20 nm radius.

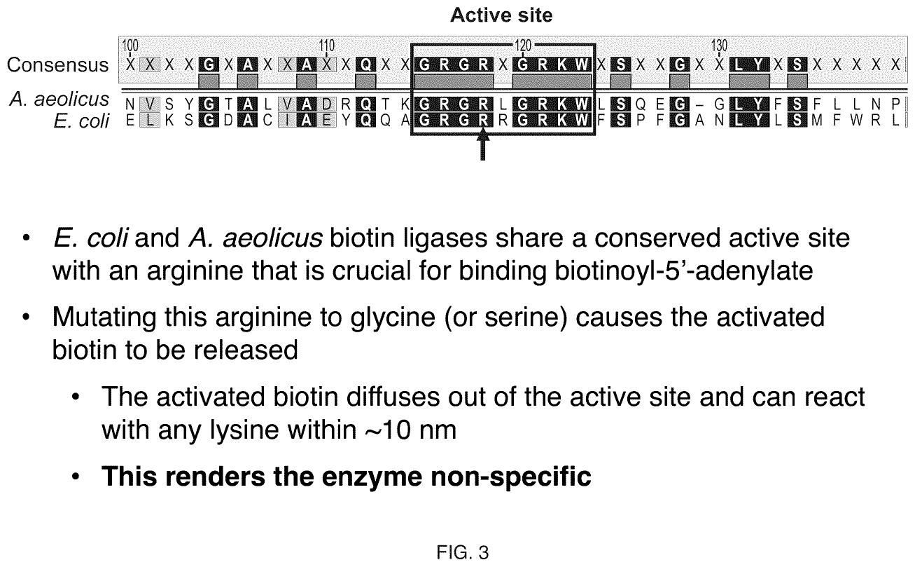

The A. aeolicus biotin ligase (aaBL) performs a similar function to E. coli BirA, biotinylating the biotin-carboxyl carrier protein subunit of acetyl-CoA carboxylase, which plays an important role in lipid metabolism. An aaBL variant with a similar active site mutation (R40G) also enables broad reactivity with proximal lysine residues. In addition, aaBL is ˜9 kDa and 100 amino acid residues smaller than BirA. FIG. 3 shows the consensus sequence and the A. aeolicus and E. coli biotin ligase sequences surrounding and including the active site. The arrow indicates the arginine that is modified to render the ligases non-specific.

### Biotin Protein Ligase Variants

The biotin protein ligases of the disclosure mediate the conjugation of biotin to a cell of interest. As shown in the Examples, it was found that selected modification improves the activity of the biotin protein ligase. Accordingly, in some embodiments, the polypeptide of the disclosure may comprise an amino acid sequence with at least 70%, 80%, 85%, 90%, 95%, 96%, 97%, 98%, 99%, or even 100% sequence identity to a sequence as set forth in SEQ ID NO: 1, wherein said amino acid sequence comprises an amino acid alteration in the active site, wherein the active site comprises the following sequence: GRGRXGRKW (SEQ ID NO: 14), wherein the X is L or P. In some embodiments, the polypeptide of the active site may comprise an amino acid sequence with at least 70%, 80%, 85%, 90%, 95%, 96%, 97%, 98%, 99%, or even 100% sequence identity to the sequence: GRGRXGRKW (SEQ ID NO: 14), wherein the X is L or P. In some embodiments, the biotin protein ligase is in A. aeolicus biotin protein ligase. In some embodiments, the active site comprises the following sequence GRGZXGRKW (SEQ ID NO: 16), where in the Z is glycine or serine. In some embodiments, mutating the arginine at position 118 and the active site results in the diffusion of activated biotin which is then able to react with lysine's within approximately 10 nm. In some embodiments, the biotin protein ligase comprises an L41P mutation in the active site of an A. Aeolicus (BioID2) biotin protein ligase.

In some embodiments, the biotin protein ligase variant reacts with an endogenous substrate on the surface of a cell. In another embodiment, the biotin protein ligase attaches and effector recognition handle on the surface of the cell. In some embodiments, the biotin protein ligase variant reacts with an exposed lysine residue on the surface of a target cell. In some embodiments, the biotin protein ligase variant has at least about 70%, 80%, 85%, 90%, 95%, 96%, 97%, 98%, 99%, or even 100% sequence identity to one of the following sequences:

BioID2
(SEQ ID NO: 17)
MFKNLIWLKEVDSTQERLKEWNVSYGTALVADRQTKGRGGLGRKW
LSQEGGLYFSFLLNPKEFENLLQLPLVLGLSVSEALEEITEIPFS
LKWPNDVYFQEKKVSGVLCELSKDKLIVGIGINVNQREIPEEIKD
RATTLYEITGKDWDRKEVLLKVLKRISENLKKFKEKSFKEFKGKI
ESKMLYLGEEVKLLGEGKITGKLVGLSEKGGALILTEEGIKEILS
GEFSLRRS;
BioID2
(Polynucleotide Sequence)
(SEQ ID NO: 18)
ATGTTTAAGAATCTAATATGGTTAAAAGAAGTGGATAGCACCCAG
GAGCGCCTGAAAGAGTGGAATGTGAGCTATGGTACTGCTTTAGTC
GCAGATCGCCAGACCAAAGGTAGAGGCGGGTTGGGCCGTAAATGG
CTGTCGCAAGAGGGAGGCCTGTACTTCTCCTTTCTGTTGAACCCG
AAAGAGTTCGAAAACCTGTTACAACTGCCGCTGGTTCTAGGTCTC
TCAGTTTCTGAGGCCCTGGAAGAAATTACCGAAATCCCGTTTAGC
CTGAAGTGGCCAAACGACGTCTATTTCCAAGAGAAAAAAGTTTCT
GGCGTACTTTGCGAACTGTCTAAGGACAAATTGATCGTGGGCATT
GGTATTAACGTGAATCAGCGTGAAATCCCGGAAGAGATCAAGGAC
CGCGCGACCACCCTGTACGAGATCACGGGCAAGGACTGGGATCGT
AAAGAAGTGCTGTTGAAGGTTCTCAAGCGTATTAGCGAGAACCTG
AAAAAGTTCAAAGAGAAGAGCTTTAAAGAGTTTAAGGGTAAGATC
GAAAGCAAGATGCTGTACTTGGGCGAGGAGGTTAAACTGCTCGGT
GAAGGCAAGATTACGGGTAAATTGGTGGGTCTGAGCGAAAAGGGT
GGCGCGCTGATCTTGACCGAAGAAGGTATTAAGGAGATCCTGTCC
GGCGAGTTCTCCCTGCGTCGTAGC;
microID
(SEQ ID NO: 19)
MFKNLIWLKEVDSTQERLKEWNVSYGTALVADRQTKGRGGLGRKW
LSQEGGLYFSFLLNPKEFENLLQLPLVLGLSVSEALEEITEIPFS
LKWPNDVYFQEKKVSGVLCELSKDKLIVGIGINVNQREIPEEIKD
RATTLYEITGKDWDRKEVLLKVLKRISENLKKFKEK;
microID
(Polynecleotide Sequence)
(SEQ ID NO: 20)
ATGTTTAAGAATCTAATATGGTTAAAAGAAGTGGACAGCACCCAA
GAACGTCTGAAGGAGTGGAACGTTAGCTATGGTACTGCTCTGGTT
GCGGATCGCCAGACCAAAGGTCGTGGTGGCCTGGGCCGTAAGTGG
CTGTCGCAAGAAGGTGGCCTCTACTTCAGCTTTCTGTTAAATCCG
AAAGAGTTTGAAAACTTGCTGCAGCTGCCGCTGGTCTTGGGTTTG
TCCGTGTCTGAGGCGCTGGAAGAGATCACGGAAATCCCGTTTTCT
CTGAAGTGGCCAAATGATGTTTATTTCCAAGAGAAGAAGGTCAGC
GGTGTTCTTTGCGAACTGTCCAAAGACAAACTGATCGTGGGCATC
GGCATTAACGTGAACCAGCGTGAAATCCCGGAAGAGATTAAAGAC
CGCGCAACCACCCTGTACGAAATTACCGGTAAAGACTGGGATCGT
AAGGAGGTTTTGTTGAAGGTGCTCAAGCGCATTAGCGAGAACCTG
AAGAAATTCAAAGAGAAA;
ultraID
(SEQ ID NO: 1)
MFKNLIWLKEVDSTQERLKEWNVSYGTALVADRQTKGRGGPGRKW
LSQEGGLYFSFLLNPKEFENLLQLPLVLGLSVSEALEEITEIPFS
LKWPNDVYFQEKKVSGVLCELSKDKLIVGIGINVNQREIPEEIKD
RATTLYEITGKDWDRKEVLLKVLKRISENLKKFKEK.
ultraID
(Polynucleotide Sequence)
(SEQ ID NO: 21)
ATGTTTAAGAATCTAATATGGTTAAAAGAAGTGGACAGCACCCAA
GAACGTCTGAAGGAGTGGAACGTTAGCTATGGTACTGCTCTGGTT
GCGGATCGCCAGACCAAAGGTCGTGGTGGCCCGGGCCGTAAGTGG
CTGTCGCAAGAAGGTGGCCTCTACTTCAGCTTTCTGTTAAATCCG
AAAGAGTTTGAAAACTTGCTGCAGCTGCCGCTGGTCTTGGGTTTG
TCCGTGTCTGAGGCGCTGGAAGAGATCACGGAAATCCCGTTTTCT
CTGAAGTGGCCAAATGATGTTTATTTCCAAGAGAAGAAGGTCAGC
GGTGTTCTTTGCGAACTGTCCAAAGACAAACTGATCGTGGGCATC
GGCATTAACGTGAACCAGCGTGAAATCCCGGAAGAGATTAAAGAC
CGCGCAACCACCCTGTACGAAATTACCGGTAAAGACTGGGATCGT
AAGGAGGTTTTGTTGAAGGTGCTCAAGCGCATTAGCGAGAACCTG
AAGAAATTCAAAGAGAAA.

In some embodiments, the biotin protein ligase comprises a truncation of the C terminus (e. g., a truncation of at least about 10, 20, 30, 40, 45, 46, 47, 48, 49, 50, 51, 52, 53, 54, 55, 56, 57, 58, 59, or 60 amino acids). In some embodiments, the truncation occurs at an amino acid residue between amino acid positions 160 and 190 of A. Aeolicus (BioID2) or a corresponding position in another biotin protein ligase (e.g., 160, 161, 162, 163, 164, 165, 166, 167, 168, 169, 170, 171, 170 to, 173, 174, 175, 176, 177, 178, 179, 180, 181, 180 to, 183, 184, 185, 186, 187, 188, 189, 190). In some embodiments the truncation occurs after amino acid position 171 of A. Aeolicus (BioID2) or a corresponding position in another biotin ligase. In some embodiments, the biotin protein ligase biotinylate's a lysine in a substrate protein (e.g., biotin carboxyl carrier protein) present on the surface of a target cell. In some embodiments, the biotin associates with a biotin binding moiety (anti-biotin antibody, avidin, streptavidin) that is bound to an agent (e.g., polypeptide, polynucleotide, or small molecule). Streptavidin binds biotin with high affinity and provides for the localization of associated agents on a cell surface decorated with biotin. In other embodiments, avidin, avidin analogues, or other molecules that bind biotin with high affinity (e.g., anti-biotin antibodies) provide for the localization of associated agents on a cell surface decorated with biotin. Avidin analogues include, but are not limited to, avidin, streptavidin, neutravidin, bradavidin II, tamavidin 2, shwanavidin, switchavidin, and zebavidin (Jain A, Cheng K. The principles and applications of avidin-based nanoparticles in drug delivery and diagnosis. J Control Release. 2017 Jan. 10; 245:27-40).

A polypeptide of the disclosure (e.g., biotin protein ligase, or fusion protein comprising the same) is capable of promoting biotinylation of a lysine residue on the surface of a cell or other substrate under conditions that are suitable for biotinylation or that are otherwise suitable for the ligase activity of the polypeptide of the disclosure. It is evident from the Examples below that the polypeptide of the disclosure is active under a range of conditions. For instance, in PBS, or Tris borate (TB) buffer at a pH of 6.0-9.0, e.g. 7.0-9.0, 7.25-8.75, such as about 7.5-8.5, over a wide range of temperatures, e.g. 0-40 degrees Celsius, such as 5-39, 10-38, 15-37 degrees Celsius, e.g. 1, 2, 3, 4, 5, 10, 12, 15, 18, 20, 22, 25, 27, 29, 31, 33, 35 or 37 degrees Celsius, about 15 degrees Celsius The polypeptide is functional in the presence of extracellular concentrations of NaCl, e.g. about 150 mM NaCl or less. However, in some embodiments, it may be preferable to perform ligation reactions in the absence of NaCl. The polypeptide of the disclosure is also active in the presence of the commonly used detergents, such as Tween 20 and Triton X-100 up to a concentration of about 2% (v/v). Moreover, the polypeptide is active in the presence of glycerol at concentrations of up to about at least 40% (v/v). Thus, in some embodiments, it may be preferable to perform ligation reactions in the presence of glycerol, e.g., about 5-50%, 10-40%, preferably about 15-30% (v/v). The skilled person would readily be able to determine other suitable conditions. In some embodiments, the polypeptide is functional in the presence of media (e.g., RPMI, DMEM, and 10% fetal bovine serum).

Thus, in some embodiments, conditions that are suitable for biotinylation and/or that are otherwise suitable for the ligase activity of the polypeptide of the disclosure includes any conditions in which contacting the biotin protein ligase of the disclosure with a target cell results in biotinylation of the target cell.

In some embodiments, contacting the biotin polypeptide ligase variant as defined herein “under conditions that are suitable for ligase activity” includes contacting said polypeptide in the presence of a chemical chaperone, e.g., a molecule that enhances or improves the reactivity of the polypeptide. In some embodiments, the chemical chaperone is TMAO (trimethylamine N-oxide). In some embodiments, the chemical chaperone, e.g., TMAO, is present in the reaction at a concentration of at least about 0.2 M, e.g., at least 0.3, 0.4, 0.5, 1.0, 1.5, 2.0 or 2.5 M, e.g. about 0.2-3.0 M, 0.5-2.0 M, 1.0-1.5 M.

In some embodiments, the polypeptide of the disclosure thus encompasses mutant forms of a reference biotin protein ligase (i.e., referred to herein as homologues, variants or derivatives) which are structurally similar to an A. Aeolicus (BioID2) biotin protein ligase or the exemplified polypeptide set forth in SEQ ID NO: 1 or contain the active site sequence (e.g., with 85%, 90%, 95%, 99% or 100% sequence identity), GRGRXGRKW (SEQ ID NO: 14), wherein the X is L or P, and are able to function as a ligase, particularly capable of promoting biotinylation under suitable conditions as defined herein. In cases where a polypeptide variant comprises mutations, e.g., deletions or insertions, relative to A. Aeolicus (BioID2) biotin protein ligase or SEQ ID NO: 1, the residues specified above are present at equivalent amino acid positions in the variant polypeptide sequence. In a preferred embodiment, deletions in the polypeptide variants of the disclosure are N-terminal and/or C-terminal truncations.

In other embodiments, a biotin protein ligase useful in the methods of the invention is derived from a eukaryote or prokaryotic organism. Possible sources for biotin protein ligases include mammalian and non-mammalian animal cells, plant cells, algae (e.g., blue-green algae), fungi, bacteria, protozoa, viruses, etc.

Thus, in some embodiments, a polypeptide variant of the present disclosure may differ from SEQ ID NO: 1 or another reference biotin protein ligase by for example 1 to 20, 1 to 15, 1 to 10, 1 to 8, 1 to 6, 1 to 5, 1 to 4, e.g., 1, 2 to 3 amino acid substitutions, insertions and/or deletions, preferably substitutions. In some embodiments, any other mutations that are present in the polypeptide (biotin protein ligase) of the present disclosure may be conservative amino acid substitutions. A conservative amino acid substitution refers to the replacement of an amino acid by another which preserves the physicochemical character of the polypeptide (e.g. D may be replaced by E or vice versa, N by Q, or L or I by V or vice versa). Thus, generally the substituting amino acid has similar properties, e.g. hydrophobicity, hydrophilicity, electronegativity, bulky side chains etc. to the amino acid being replaced. Isomers of the native L-amino acid e.g. D-amino acids may be incorporated.

Sequence identity may be determined by any suitable means known in the art, e.g. using the SWISS-PROT protein sequence databank using FASTA pep-cmp with a variable pamfactor, and gap creation penalty set at 12.0 and gap extension penalty set at 4.0, and a window of 2 amino acids. Other programs for determining amino acid sequence identity include the BestFit program of the Genetics Computer Group (GCG) Version 10 Software package from the University of Wisconsin. The program uses the local homology algorithm of Smith and Waterman with the default values: Gap creation penalty-8, Gap extension penalty=2, Average match=2.912, Average mismatch=−2.003. In some embodiments, said comparison is made over the full length of the sequence, but may be made over a smaller window of comparison, e.g. less than 100, 80 or 50 contiguous amino acids.

In some embodiments, such sequence identity-related proteins (polypeptide variants) are functionally equivalent to the polypeptides which are set forth herein (e.g. SEQ ID NO. 1 or another biotin protein ligase delineated herein). As referred to herein, “functional equivalence” refers to variants of the polypeptide (e.g. ligase) of the disclosure discussed above that may show increased or reduced efficacy in the ligation reaction (e.g. lower yield of reaction, lower reaction rate or activity in a limited range of reaction conditions (e.g. narrower temperature range, such as 10-30 degrees Celsius etc.)) relative to the parent molecule (i.e. the molecule with which it shows sequence homology), but preferably are as efficient or are more efficient.

A mutant or variant polypeptide of the disclosure with ligase or catalytic activity that is “equivalent” to the ligase or catalytic activity of a polypeptide comprising or consisting of an amino acid sequence as set forth in SEQ ID NO: 1 may have ligase or catalytic activity that is similar (i.e. comparable) to the ligase or catalytic activity of a polypeptide comprising or consisting of an amino acid sequence as set forth in SEQ ID NO: 1, i.e. such that the practical applications of the peptide ligase are not significantly affected, e.g. within a margin of experimental error. Thus, an equivalent ligase or catalytic activity means that the mutant or variant polypeptide of the disclosure is capable of promoting the formation of an isopeptide bond between the biotin protein ligase s of the disclosure with a similar reaction rate and/or yield of reaction to a polypeptide comprising or consisting of an amino acid sequence as set forth in SEQ ID NO: 1 under the same conditions.

The ligase or catalytic activity of different biotin protein ligase polypeptides (e.g. SEQ ID NO: 1) measured under the same reaction conditions, e.g. temperature, substrates (i.e. biotin protein ligase sequences) and their concentration, buffer, salt etc. as exemplified herein, can be readily compared to determine whether the ligase or catalytic activity for each protein is higher, lower or equivalent.

Thus, the ligase or catalytic activity of the variant (e.g. mutant) biotin protein ligase may be at least 60%, e.g. at least 70, 75, 80, 85 or 90% of the ligase or catalytic activity of a polypeptide comprising or consisting of an amino acid sequence as set forth in SEQ ID NO: 1, such as at least 91, 92, 93, 94, 95, 96, 97, 98 or 99% of the ligase or catalytic activity of a polypeptide comprising or consisting of an amino acid sequence as set forth in SEQ ID NO: 1. Alternatively viewed, the ligase or catalytic activity of the mutant polypeptide may be no more than 40% lower than the ligase or catalytic activity of a polypeptide comprising or consisting of an amino acid sequence as set forth in SEQ ID NO: 1, e.g. no more than 35, 30, 25 or 20% lower than the ligase or catalytic activity of a polypeptide comprising or consisting of an amino acid sequence as set forth in SEQ ID NO: 1, such as no more than 10, 9, 8, 7, 6, 5, 4, 3, 2 or 1% lower than the ligase or catalytic activity of a polypeptide comprising or consisting of an amino acid sequence as set forth in SEQ ID NO: 1.

In some embodiments, the ligase or catalytic activity of the variant (e.g., mutant) polypeptide may be assessed by measuring the biotinylation of a substrate protein. In embodiments, a biotin protein ligase variant has a yield of reaction of at least about 50%-100% (e.g., 50%, 60%, 70%, 80%, 90%, 95%, 96% 97% 98% 99% or 100%).

Hence, any modification or combination of modifications may be made to SEQ ID NO: 1 to produce a variant polypeptide (biotin protein ligase variant) of the disclosure, provided that the variant polypeptide comprises an amino acid mutation within the active site of the ligase and/or a truncation at the C terminus. In some embodiments, SEQ ID NO: 1 comprises at least one (e.g., 2, 3 or 4) other amino acid residue(s) alteration, but nevertheless retains the functional characteristics defined above, i.e. it results in a biotin protein ligase capable of biotinylation and optionally has an equivalent or higher yield of reaction, reaction rate, temperature and/or buffer range relative to a polypeptide having an amino acid sequence as set forth in SEQ ID NO: 1.

An equivalent or corresponding amino acid position is determined by reference to an A. Aeolicus (BioID2) biotin protein ligase sequence or the amino acid sequence of SEQ ID NO: 1. The homologous or corresponding position can be readily deduced by lining up the sequence of the homologue (mutant, variant or derivative) polypeptide and the sequence of SEQ ID NO: 1 based on the homology or identity between the sequences, for example using a BLAST algorithm.

In some embodiments, a biotin protein ligase variant described herein is fused to a linker. The term “linker” as used herein generally refers to a peptide. There is no standard definition regarding the size boundaries between what is meant by peptide, but typically a peptide may be viewed as comprising between 2-20 amino acids. Accordingly, a polypeptide may be viewed as comprising at least 40 amino acids, at least 50, 60, 70 or 80 amino acids. Thus, a linker as defined herein may be viewed as comprising at least 3, 5, 6, 7, 8, 9, 10, 11, or 12 amino acids, e.g. 12-39 amino acids, such as e.g. 13-35, 14-34, 15-33, 16-31, 17-30 amino acids in length, e.g. it may comprise or consist of 12, 13, 14, 15, 16, 17, 18, 19, 20, 21, 22 or 23 amino acids.

### Diagnostic, Detectable, and Therapeutic Agents Targeted to Markers on the Cell Surface

The disclosure provides a new type of targeting system based around an enzyme, biotin protein ligase. Rather than attaching a cargo (label, drug, protein, oligonucleotide) directly to a ligand (antibody, nanobody, aptamer, etc.) that targets a cell surface marker, the disclosure employs an enzyme (e.g., biotin protein ligase) attached to a ligand (e.g., antibody, antigen-binding fragment thereof, nanobody, aptamer) that binds a cell surface marker (tumor marker, immune cell marker). Once bound to the cell surface, the biotin protein ligase iteratively attaches biotin or a biotin variant to the surface of the target cell. Biotin or the biotin variant is then bound by a biotin binding moiety (e.g., streptavidin, avidin, an anti-biotin antibody, or an antigen binding fragment thereof) attached to a cargo (e.g., fluorophore, therapeutic agent, toxin). This approach takes advantage of the catalytic nature of enzymes for signal amplification and introduces opportunities for additional layers of precision (e.g., using an enzyme that is only active in a specific context, and enzymes split into two sections, or a multi-enzyme logic gate). Accordingly, this disclosure provides a wide array of uses for this technology in both clinical and research settings. In some embodiments, the biotin protein ligases are used to target agents as payloads, where the agent is bound to a biotin binding moiety (e.g., anti-biotin antibody, antigen-binding fragment thereof, streptavidin, avidin) which is subsequently bound to biotin that has been ligated to the cell surface.

Agents useful in the methods described herein include polypeptides, polynucleotides, and small compounds. Specific agents useful in the methods described herein include nucleic acid molecules (e.g., DNA, RNA, DNA-RNA hybrids, siRNAs, antisense RNAs, mRNAs, aptamers), proteins, peptides, small-molecule organic compounds, fluorophores, polysaccharides, nanoparticles, nanotubes, polymers, viruses, virus-like particles or any combination of these. In one embodiment, agents are conjugated to streptavidin, avidin, avidin analogues or anti-biotin antibody, which then binds to biotin that has been ligated to the cell surface.

In some embodiments, the agent is a label, e.g. a radiolabel, a fluorescent label, luminescent label, a chromophore label, as well as the substances and enzymes which generate a detectable substrate, e.g. horse radish peroxidase, luciferase or alkaline phosphatase.

### Cytokines

The immune system is skilled in communication and designed to respond quickly, specifically and globally to protect an organism against foreign invaders and disease. The cytokine superfamily of proteins is an integral part of the signaling network between cells and is essential in generating and regulating the immune system. Cytokines are small soluble factors with pleiotropic functions that are produced by many cell types as part of a gene expression pattern that can influence and regulate the function of the immune system.

Exemplary cytokines include, but are not limited to, IL-1-like, IL-1 alpha, IL-1 beta, IL-18, IL-2, IL-4, IL-7, IL-9, IL-3, IL-5, IL-10, IL-12, G-CSF, leukemia inhibitory factor, interferon alpha, interferon beta, interferon gamma, CD 154, TNF-alpha, TNF-beta, CD 70, CD 153, Ox40L, TGF beta, stem cell factor, and macrophage stimulating factor.

### Chemotherapeutics

Chemotherapeutics are those agents that are useful for the treatment of cancer. Exemplary chemotherapeutic classes include: alkylating agents, anthracyclines, cytoskeletal disruptors (taxanes), histone deacetylase inhibitors, kinase inhibitors, platinum-based agents, retinoids, Vinca alkaloids. Specific chemotherapeutic agents include the following: Actinomycin

All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine, and Vindesine.

### Aptamers

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. Desirably, the aptamers are small, approximately ˜15 KD. The aptamers are isolated from libraries consisting of some 1014-1015 random oligonucleotide sequences by a procedure termed SELEX (systematic evolution of ligands by exponential enrichment). See Tuerk et al., Science, 249:505-510, 1990; Green et al., Methods Enzymology. 75-86, 1991; Gold et al., Annu. Rev. Biochem., 64:763-797, 1995; Uphoff et al., Curr. Opin. Struct. Biol., 6:281-288, 1996. Methods of generating aptamers are known in the art and are described, for example, in U.S. Pat. Nos. 6,344,318, 6,331,398, 6,110,900, 5,817,785, 5,756,291, 5,696,249, 5,670,637, 5,637,461, 5,595,877, 5,527,894, 5,496,938, 5,475,096, 5,270,163, and in U.S. Patent Application Publication Nos. 20040241731, 20030198989, 20030157487, and 20020172962.

### Target Cells

Target cells for the biotin protein ligase variants described herein may be prokaryotic or eukaryotic cells. In some embodiments, the cell is a prokaryotic cell, e.g., a bacterial cell. In other embodiments, the target is a mammalian cell (e.g., human, rodent, canine, feline, murine, equine, bovine, or other mammalian livestock). In some embodiments, the agent used to decorate the target cell is a compound or molecule which has a therapeutic or prophylactic effect, e.g., growth factor, antitumour agent (e.g. a radioactive compound or isotope), chemotherapeutic, cytokine, toxin, antibiotic, antiviral, vaccine, or oligonucleotide.

Exemplary cells that might be targeted include, but are not limited to, T cells, Car-T cells B cells, NK cells, and other immune cells. In one embodiment, a cell of the invention is ablated with the cytotoxin. In one embodiment, cells targeted for ablation include neoplastic cells, tumor cells, activated T cells, early activated memory T cells (e.g., in lupus, or another autoimmune disease, memory T/B cells, memory CD4 positive T cells carrying HIV, exhausted antitumor T cells, tumor, resident macrophages, Tregs, MDSCs). In another embodiment, biotin protein ligases of the invention may be used, to specifically activate immune cells. In another embodiment, biotin protein ligases of the invention may be used to target Car-T cells. Advantageously, this approach could be used, to generate a universal Car-T cell, which would include biotin decorating it's surface. In another embodiment, biotin protein ligases described herein may be used to target a Crispr system to a particular cell type.

### Method of Treatment

In one embodiment, the present invention provides a method of treating a disease (e.g., neoplasia, autoimmune disorder) comprising the step of administering to the subject an effective amount of a fusion protein comprising a biotin protein ligase and a targeting moiety (e.g., antibody) and a therapeutic agent conjugated to a biotin binding moiety (e.g., anti-biotin antibody, streptavidin, avidin, or variance of any of the aforementioned) preferably as part of a composition additionally comprising a pharmaceutically acceptable carrier. Other embodiments include any of the methods herein wherein the subject is identified as in need of the indicated treatment.

### Recombinant Polypeptides

Fusion proteins comprising a biotin protein ligase may be fused or conjugated with another polypeptide using recombinant techniques as discussed below, i.e. as a recombinant or synthetic protein or polypeptide. Biotin protein ligases may be fused to any protein or peptide of interest. The protein may be derived or obtained from any suitable source. For instance, the protein may be in vitro translated or purified from biological and clinical samples, e.g. any cell or tissue sample of an organism (eukaryotic, prokaryotic), or any body fluid or preparation derived therefrom, as well as samples such as cell cultures, cell preparations, cell lysates etc. Proteins may be derived or obtained, e.g., purified from environmental samples, e.g. soil and water samples or food samples are also included. The samples may be freshly prepared or they may be prior-treated in any convenient way e.g. for storage.

As noted above the protein may be produced recombinantly and thus the nucleic acid molecules encoding said proteins may be derived or obtained from any suitable source, e.g., any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa, viruses etc. In some embodiments, the proteins may be synthetic proteins. For example, the peptide and polypeptide (proteins) disclosed herein may be produced by chemical synthesis, such as solid-phase peptide synthesis.

The position of the biotin protein ligase within a recombinant protein is not particularly important. Thus, in some embodiments the biotin protein ligase may be located at the N-terminus or C-terminus of the recombinant or synthetic polypeptide. In some embodiments, the biotin protein ligase may be located internally within the recombinant or synthetic polypeptide. Thus, in some embodiments the biotin protein ligase may be viewed as an N-terminal, C-terminal or internal domain of the recombinant or synthetic polypeptide.

In an embodiment, the ligase is preferably located at the N-terminus or C-terminus of the recombinant or synthetic polypeptide. In some embodiments, the ligase may be located internally within the recombinant or synthetic polypeptide. Thus, in some embodiments the biotin protein ligase may be viewed as an N-terminal, C-terminal or internal domain of the recombinant or synthetic polypeptide.

In one embodiment, a SpyCatcher recombinant system is used (BioRad, Hercules, California). SpyCatcher3 (H-SpyC3) is a 15.2 kDa protein that forms a stable covalent isopeptide bond with a second protein that includes a SpyTag (Keeble et al., Approaching infinite affinity through engineering of peptide-protein interaction, Biochemistry 116 (52), 26523-26533, Dec. 10, 2019). For example, one exemplary Spycatcher and one exemplary Spy Tag sequence are:

SpyCatcher-ultraID
(polypeptide)
(SEQ ID NO: 22)
MVTTLSGLSGEQGPSGDMTTEEDSATHIKFSKRDEDGRELAGATM
ELRDSSGKTISTWISDGHVKDFYLYPGKYTFVETAAPDGYEVATP
IEFTVNEDGQVTVDGEATEGDAHTGSSGSGGGSGGGSGGGSMFKN
LIWLKEVDSTQERLKEWNVSYGTALVADRQTKGRGGPGRKWLSQE
GGLYFSFLLNPKEFENLLQLPLVLGLSVSEALEEITEIPFSLKWP
NDVYFQEKKVSGVLCELSKDKLIVGIGINVNQREIPEEIKDRATT
LYEITGKDWDRKEVLLKVLKRISENLKKFKEK
SpyCatcher-ultraID
(polynucleotide)
(SEQ ID NO: 23)
ATGGTAACTACACTATCAGGATTATCTGGCGAGCAGGGTCCGAGC
GGCGATATGACCACTGAGGAAGACTCTGCCACGCATATCAAATTT
TCGAAGCGCGACGAAGACGGTCGTGAACTGGCGGGTGCTACGATG
GAATTGCGTGATAGCAGCGGTAAGACCATTTCCACCTGGATTAGC
GACGGCCACGTGAAAGACTTCTACCTGTATCCAGGTAAATATACC
TTTGTTGAAACCGCGGCACCGGATGGCTACGAGGTGGCGACTCCG
ATCGAGTTCACGGTTAACGAAGATGGTCAAGTAACAGTAGATGGT
GAAGCTACAGAAGGTGATGCACATACAGGTTCAAGTGGAAGTGGA
GGTGGCAGTGGCGGCGGCAGTGGTGGAGGCAGTATGTTTAAGAAT
CTAATATGGTTAAAAGAAGTGGACAGCACCCAAGAACGTCTGAAG
GAGTGGAACGTTAGCTATGGTACTGCTCTGGTTGCGGATCGCCAG
ACCAAAGGTCGTGGTGGCCCGGGCCGTAAGTGGCTGTCGCAAGAA
GGTGGCCTCTACTTCAGCTTTCTGTTAAATCCGAAAGAGTTTGAA
AACTTGCTGCAGCTGCCGCTGGTCTTGGGTTTGTCCGTGTCTGAG
GCGCTGGAAGAGATCACGGAAATCCCGTTTTCTCTGAAGTGGCCA
AATGATGTTTATTTCCAAGAGAAGAAGGTCAGCGGTGTTCTTTGC
GAACTGTCCAAAGACAAACTGATCGTGGGCATCGGCATTAACGTG
AACCAGCGTGAAATCCCGGAAGAGATTAAAGACCGCGCAACCACC
CTGTACGAAATTACCGGTAAAGACTGGGATCGTAAGGAGGTTTTG
TTGAAGGTGCTCAAGCGCATTAGCGAGAACCTGAAGAAATTCAAA
GAGAAA
anti-hCD4 nanobody-SpyTag
(human CD4) (polypeptide)
(SEQ ID NO: 24)
EVQLVQSGAELVKPGASVKLSCKVSDYNIRRTYMHWVRQRPGKGL
EWIGRIDPANGNTIYGEKFKSKATLTADTSSNTAYMQLSQLKSDD
TAIYYCAIGVQYLDYWGQGTTVTVSSGGGGSGGGGSGGGGSDIVL
TQSPALAVSPGERVTISCRATESVSTLIHWYQQRPGQQPKLLIYL
TSHLDSGVPARFSGSGSGTDFTLTIDPVEADDTATYYCQQTWNDP
WTFGGGTKLELKRGGGSGGGSGGGSRGVPHIVMVDAYKRYK
anti-hCD4 nanobody-SpyTag
(human CD4) (polynucleotide)
(SEQ ID NO: 25)
GAGGTTCAGCTAGTACAATCAGGAGCTGAATTGGTTAAGCCGGGC
GCTTCCGTCAAGCTGAGCTGCAAAGTTTCGGACTATAACATTCGC
CGTACCTACATGCATTGGGTTCGTCAGCGTCCGGGTAAGGGTTTG
GAATGGATTGGTCGTATTGATCCGGCTAATGGAAACACCATCTAC
GGCGAGAAATTCAAAAGCAAAGCGACCCTGACTGCGGACACCTCT
AGCAATACCGCGTATATGCAGCTGTCTCAGCTTAAGAGCGATGAC
ACGGCGATTTATTACTGCGCAATCGGCGTGCAGTATCTCGACTAC
TGGGGTCAAGGTACGACCGTGACCGTCTCCAGCGGCGGCGGTGGC
TCTGGTGGCGGTGGCTCCGGTGGTGGCGGTTCCGATATTGTACTG
ACGCAGAGCCCGGCGCTGGCCGTTTCCCCGGGTGAACGTGTTACC
ATCAGCTGTCGCGCAACTGAGAGCGTGTCGACCCTGATCCACTGG
TATCAGCAAAGACCAGGTCAACAACCGAAATTGTTAATCTACCTG
ACCAGCCACCTGGACAGCGGGGTGCCGGCCCGTTTTAGCGGTTCT
GGCAGCGGCACCGATTTCACCCTGACCATCGATCCGGTGGAAGCA
GACGACACTGCGACGTACTACTGCCAGCAAACCTGGAACGATCCT
TGGACCTTTGGTGGTGGTACAAAATTGGAGCTGAAGCGCGGAGGT
GGCAGTGGCGGCGGCAGTGGTGGAGGCAGTAGGGGAGTTCCCCAC
ATAGTAATGGTCGATGCGTATAAGCGCTATAAG

In one embodiment, the biotin protein ligase is fused to a signal polypeptide (e.g., for cell localization or cell export) such as mRID or NusA. For example, one exemplary sequence is:

mRID-ultraID (polypeptide)
(SEQ ID NO: 26)
MATLQESEVKVDGEQKLSKNELKRRLKAEKKLAEKEAKQKELSEK
QLNQTASAPNHTADNGVGAEEETLDDDDDDSGENLYFQGMFKNLI
WLKEVDSTQERLKEWNVSYGTALVADRQTKGRGGPGRKWLSQEGG
LYFSFLLNPKEFENLLQLPLVLGLSVSEALEEITEIPFSLKWPND
VYFQEKKVSGVLCELSKDKLIVGIGINVNQREIPEEIKDRATTLY
EITGKDWDRKEVLLKVLKRISENLKKFKEK
mRID-ultraID (polynucleotide)
(SEQ ID NO: 27)
GGCTACACTACAAGAGTCAGAAGTAAAAGTGGATGGCGAACAGAA
GTTGTCCAAAAACGAATTAAAGCGCCGTCTGAAAGCGGAGAAGAA
ATTGGCTGAGAAGGAGGCCAAACAAAAAGAACTGTCTGAAAAGCA
ACTGAATCAGACCGCGAGCGCACCGAACCACACCGCGGATAATGG
CGTTGGTGCGGAGGAAGAGACGCTGGATGACGACGACGATGACAG
CGGTGAGAACCTGTACTTCCAGGGAATGTTTAAGAATCTAATATG
GTTAAAAGAAGTGGACAGCACCCAAGAACGTCTGAAGGAGTGGAA
CGTTAGCTATGGTACTGCTCTGGTTGCGGATCGCCAGACCAAAGG
TCGTGGTGGCCCGGGCCGTAAGTGGCTGTCGCAAGAAGGTGGCCT
CTACTTCAGCTTTCTGTTAAATCCGAAAGAGTTTGAAAACTTGCT
GCAGCTGCCGCTGGTCTTGGGTTTGTCCGTGTCTGAGGCGCTGGA
AGAGATCACGGAAATCCCGTTTTCTCTGAAGTGGCCAAATGATGT
TTATTTCCAAGAGAAGAAGGTCAGCGGTGTTCTTTGCGAACTGTC
CAAAGACAAACTGATCGTGGGCATCGGCATTAACGTGAACCAGCG
TGAAATCCCGGAAGAGATTAAAGACCGCGCAACCACCCTGTACGA
AATTACCGGTAAAGACTGGGATCGTAAGGAGGTTTTGTTGAAGGT
GCTCAAGCGCATTAGCGAGAACCTGAAGAAATTCAAAGAGAAA

In one embodiment, the biotin protein ligase is separated from the polypeptide by a protease cleavage site. In some embodiments, the protease cleavage site is recognized by, for example, furin, Tobacco Etch Virus (TEV), Rhinovirus 3C, Enterokinase, Factor Xa, or other protease. Some exemplary sequences are:

SpyCatcher-TEV protease
(polypeptide)
(SEQ ID NO: 28)
MVTTLSGLSGEQGPSGDMTTEEDSATHIKFSKRDEDGRELAGATM
ELRDSSGKTISTWISDGHVKDFYLYPGKYTFVETAAPDGYEVATP
IEFTVNEDGQVTVDGEATEGDAHTGSSGSGGGSGGGSGGGSGESL
FKGPRDYNPISSTICHLTNESDGHTTSLYGIGFGPFIITNKHLFR
RNNGTLLVQSLHGVFKVKNTTTLQQHLIDGRDMIIIRMPKDFPPF
PQKLKFREPQREERICLVTTNFQTKSMSSMVSDTSCTFPSSDGIF
WKHWIQTKDGQCGSPLVSTRDGFIVGIHSASNFTNTNNYFTSVPK
NFMELLTNQEAQQWVSGWRLNADSVLWGGHKVFMSKPEEPFQPVK
EATQLMNELVYSQ
SpyCatcher-TEV protease (polynucleotide)
(SEQ ID NO: 29)
ATGGTAACTACACTATCAGGATTATCTGGCGAGCAGGGTCCGAGC
GGCGATATGACCACTGAGGAAGACTCTGCCACGCATATCAAATTT
TCGAAGCGCGACGAAGACGGTCGTGAACTGGCGGGTGCTACGATG
GAATTGCGTGATAGCAGCGGTAAGACCATTTCCACCTGGATTAGC
GACGGCCACGTGAAAGACTTCTACCTGTATCCAGGTAAATATACC
TTTGTTGAAACCGCGGCACCGGATGGCTACGAGGTGGCGACTCCG
ATCGAGTTCACGGTTAACGAAGATGGTCAAGTAACAGTAGATGGT
GAAGCTACAGAAGGTGATGCACATACAGGTTCAAGTGGAAGTGGA
GGTGGCAGTGGCGGCGGCAGTGGTGGAGGCAGTGGAGAATCACTA
TTTAAAGGGCCCAGGGATTACAATCCGATTAGCTCCACCATCTGC
CACCTGACCAACGAGAGCGACGGCCACACCACTTCCTTATACGGC
ATCGGCTTTGGTCCGTTCATTATCACCAACAAGCACTTGTTTCGC
CGTAACAATGGTACATTGTTGGTCCAGAGCTTGCATGGTGTTTTC
AAAGTTAAGAACACCACGACGCTGCAACAACACCTGATTGATGGC
CGTGATATGATCATCATCAGAATGCCGAAGGACTTCCCGCCTTTC
CCGCAGAAACTGAAATTTCGTGAGCCGCAGCGTGAAGAGCGCATT
TGCCTGGTCACCACCAACTTCCAGACGAAAAGCATGTCTAGCATG
GTTTCCGATACCTCATGTACCTTTCCGAGCTCGGACGGCATCTTT
TGGAAACATTGGATTCAGACCAAGGACGGCCAATGCGGTTCCCCG
CTCGTATCTACTCGCGACGGCTTCATCGTGGGTATTCATAGCGCA
AGCAATTTCACCAATACCAACAACTATTTCACGTCTGTGCCAAAG
AACTTTATGGAACTTCTGACCAACCAAGAAGCGCAGCAATGGGTT
AGCGGTTGGCGTCTGAATGCTGATAGCGTGCTGTGGGGTGGTCAC
AAAGTGTTCATGTCGAAACCGGAAGAACCGTTTCAACCGGTGAAG
GAGGCGACCCAGCTGATGAACGAGCTGGTTTATAGCCAG
SpyCatcher-ultraID-TEV site-Cterm
(polypeptide)
(SEQ ID NO: 30)
MHHHHHHMVTTLSGLSGEQGPSGDMTTEEDSATHIKFSKRDEDGR
ELAGATMELRDSSGKTISTWISDGHVKDFYLYPGKYTFVETAAPD
GYEVATPIEFTVNEDGQVTVDGEATEGDAHTGSSGSGGGSGGGSG
GGSMFKNLIWLKEVDSTQERLKEWNVSYGTALVADRQTKGRGGPG
RKWLSQEGGLYFSFLLNPKEFENLLQLPLVLGLSVSEALEEITEI
PFSLKWPNDVYFQEKKVSGVLCELSKDKLIVGIGINVNQREIPEE
IKDRATTLYEITGKDWDRKEVLLKVLKRISENLKKFKEKENLYFQ
GSFKEFKGKIESKMLYLGEEVKLLGEGKITGKLVGLSEKGGALIL
TEEGIKEILSGEFSLRRSGGS
SpyCatcher-ultraID-TEV site-Cterm
(polynucleotide)
(SEQ ID NO: 31)
ATGGTAACTACACTATCAGGATTATCTGGCGAGCAGGGTCCGAGC
GGCGATATGACCACTGAGGAAGACTCTGCCACGCATATCAAATTT
TCGAAGCGCGACGAAGACGGTCGTGAACTGGCGGGTGCTACGATG
GAATTGCGTGATAGCAGCGGTAAGACCATTTCCACCTGGATTAGC
GACGGCCACGTGAAAGACTTCTACCTGTATCCAGGTAAATATACC
TTTGTTGAAACCGCGGCACCGGATGGCTACGAGGTGGCGACTCCG
ATCGAGTTCACGGTTAACGAAGATGGTCAAGTAACAGTAGATGGT
GAAGCTACAGAAGGTGATGCACATACAGGTTCAAGTGGAAGTGGA
GGTGGCAGTGGCGGCGGCAGTGGTGGAGGCAGTATGTTTAAGAAT
CTAATATGGTTAAAAGAAGTGGACAGCACCCAAGAACGTCTGAAG
GAGTGGAACGTTAGCTATGGTACTGCTCTGGTTGCGGATCGCCAG
ACCAAAGGTCGTGGTGGCCCGGGCCGTAAGTGGCTGTCGCAAGAA
GGTGGCCTCTACTTCAGCTTTCTGTTAAATCCGAAAGAGTTTGAA
AACTTGCTGCAGCTGCCGCTGGTCTTGGGTTTGTCCGTGTCTGAG
GCGCTGGAAGAGATCACGGAAATCCCGTTTTCTCTGAAGTGGCCA
AATGATGTTTATTTCCAAGAGAAGAAGGTCAGCGGTGTTCTTTGC
GAACTGTCCAAAGACAAACTGATCGTGGGCATCGGCATTAACGTG
AACCAGCGTGAAATCCCGGAAGAGATTAAAGACCGCGCAACCACC
CTGTACGAAATTACCGGTAAAGACTGGGATCGTAAGGAGGTTTTG
TTGAAGGTGCTCAAGCGCATTAGCGAGAACCTGAAGAAATTCAAA
GAGAAAGAAAATCTATATTTTCAAGGATCATTTAAAGAATTCAAG
GGCAAGATCGAAAGCAAAATGCTGTACCTGGGCGAGGAAGTGAAA
CTGCTGGGTGAGGGCAAGATCACGGGTAAGTTGGTTGGTCTCTCT
GAAAAAGGCGGTGCGCTGATTTTGACCGAAGAGGGTATTAAAGAG
ATCTTATCCGGCGAGTTCAGCCTGCGCCGTAGCGGTGGTTCG

In some embodiments, it may be useful to include one or more spacers, e.g. a peptide spacer, between the polypeptide to be joined or conjugated with the biotin protein ligase. Thus, the polypeptide and the biotin protein ligase may be fused directly to each other, or they may be linked indirectly by means of one or more spacer sequences. Thus, a spacer sequence may interspace or separate two or more individual parts of the recombinant or synthetic polypeptide. In some embodiments, a spacer may be N-terminal or C-terminal to the biotin protein ligase. In some embodiments, spacers may be at both sides of the biotin protein ligase.

The precise nature of the spacer sequence is not critical, and it may be of variable length and/or sequence, for example it may have 1-40, more particularly 2-20, 1-15, 1-12, 1-10, 1-8, or 1-6 residues, e.g., 6, 7, 8, 9, 10 or more residues. By way of representative example, the spacer sequence, if present, may have 1-15, 1-12, 1-10, 1-8 or 1-6 residues, etc. The nature of the residues is not critical, and they may for example be any amino acid, e.g., a neutral amino acid, or an aliphatic amino acid, or alternatively they may be hydrophobic, or polar or charged or structure-forming e.g., proline. In some preferred embodiments, the linker is a serine and/or glycine-rich sequence, preferably comprising at least 6 amino acid residues, e.g., 6, 7 or 8 residues.

Exemplary spacer sequences thus include any single amino acid residue, e.g., S, G, L, V, P, R, H, M, A or E or a di-, tri-tetra-penta- or hexa-peptide composed of one or more of such residues.

Some exemplary sequences are:

SpyCatcher-ultraID-TEV site-GGGSx1-Cterm
(polypeptide)
(SEQ ID NO: 32)
MVTTLSGLSGEQGPSGDMTTEEDSATHIKFSKRDEDGRELAGATM
ELRDSSGKTISTWISDGHVKDFYLYPGKYTFVETAAPDGYEVATP
IEFTVNEDGQVTVDGEATEGDAHTGSSGSGGGSGGGSGGGSMFKN
LIWLKEVDSTQERLKEWNVSYGTALVADRQTKGRGGPGRKWLSQE
GGLYFSFLLNPKEFENLLQLPLVLGLSVSEALEEITEIPFSLKWP
NDVYFQEKKVSGVLCELSKDKLIVGIGINVNQREIPEEIKDRATT
LYEITGKDWDRKEVLLKVLKRISENLKKFKEKENLYFQGGGGSSF
KEFKGKIESKMLYLGEEVKLLGEGKITGKLVGLSEKGGALILTEE
GIKEILSGEFSLRRSGGS
SpyCatcher-ultraID-TEV site-GGGSx1-Cterm
(polynucleotide)
(SEQ ID NO: 33)
ATGGTAACTACACTATCAGGATTATCTGGCGAGCAGGGTCCGAGC
GGCGATATGACCACTGAGGAAGACTCTGCCACGCATATCAAATTT
TCGAAGCGCGACGAAGACGGTCGTGAACTGGCGGGTGCTACGATG
GAATTGCGTGATAGCAGCGGTAAGACCATTTCCACCTGGATTAGC
GACGGCCACGTGAAAGACTTCTACCTGTATCCAGGTAAATATACC
TTTGTTGAAACCGCGGCACCGGATGGCTACGAGGTGGCGACTCCG
ATCGAGTTCACGGTTAACGAAGATGGTCAAGTAACAGTAGATGGT
GAAGCTACAGAAGGTGATGCACATACAGGTTCAAGTGGAAGTGGA
GGTGGCAGTGGCGGCGGCAGTGGTGGAGGCAGTATGTTTAAGAAT
CTAATATGGTTAAAAGAAGTGGACAGCACCCAAGAACGTCTGAAG
GAGTGGAACGTTAGCTATGGTACTGCTCTGGTTGCGGATCGCCAG
ACCAAAGGTCGTGGTGGCCCGGGCCGTAAGTGGCTGTCGCAAGAA
GGTGGCCTCTACTTCAGCTTTCTGTTAAATCCGAAAGAGTTTGAA
AACTTGCTGCAGCTGCCGCTGGTCTTGGGTTTGTCCGTGTCTGAG
GCGCTGGAAGAGATCACGGAAATCCCGTTTTCTCTGAAGTGGCCA
AATGATGTTTATTTCCAAGAGAAGAAGGTCAGCGGTGTTCTTTGC
GAACTGTCCAAAGACAAACTGATCGTGGGCATCGGCATTAACGTG
AACCAGCGTGAAATCCCGGAAGAGATTAAAGACCGCGCAACCACC
CTGTACGAAATTACCGGTAAAGACTGGGATCGTAAGGAGGTTTTG
TTGAAGGTGCTCAAGCGCATTAGCGAGAACCTGAAGAAATTCAAA
GAGAAAGAAAATCTATATTTTCAAGGAGGAGGTGGCAGTTCATTT
AAAGAATTCAAGGGCAAGATCGAAAGCAAAATGCTGTACCTGGGC
GAGGAAGTGAAACTGCTGGGTGAGGGCAAGATCACGGGTAAGTTG
GTTGGTCTCTCTGAAAAAGGCGGTGCGCTGATTTTGACCGAAGAG
GGTATTAAAGAGATCTTATCCGGCGAGTTCAGCCTGCGCCGTAGC
GGTGGTTCG
SpyCatcher-ultraID-TEV site-GGGSx2-Cterm
(polypeptide)
(SEQ ID NO: 34)
MFKNLIWLKEVDSTQERLKEWNVSYGTALVADRQTKGRGGPGRKW
LSQEGGLYFSFLLNPKEFENLLQLPLVLGLSVSEALEEITEIPFS
LKWPNDVYFQEKKVSGVLCELSKDKLIVGIGINVNQREIPEEIKD
RATTLYEITGKDWDRKEVLLKVLKRISENLKKFKEKENLYFQGGG
GSGGGSSFKEFKGKIESKMLYLGEEVKLLGEGKITGKLVGLSEKG
GALILTEEGIKEILSGEFSLRRSGGS
SpyCatcher-ultraID-TEV site-GGGSx2-Cterm
(polynucleotide)
(SEQ ID NO: 35)
ATGTTTAAGAATCTAATATGGTTAAAAGAAGTGGACAGCACCCAA
GAACGTCTGAAGGAGTGGAACGTTAGCTATGGTACTGCTCTGGTT
GCGGATCGCCAGACCAAAGGTCGTGGTGGCCCGGGCCGTAAGTGG
CTGTCGCAAGAAGGTGGCCTCTACTTCAGCTTTCTGTTAAATCCG
AAAGAGTTTGAAAACTTGCTGCAGCTGCCGCTGGTCTTGGGTTTG
TCCGTGTCTGAGGCGCTGGAAGAGATCACGGAAATCCCGTTTTCT
CTGAAGTGGCCAAATGATGTTTATTTCCAAGAGAAGAAGGTCAGC
GGTGTTCTTTGCGAACTGTCCAAAGACAAACTGATCGTGGGCATC
GGCATTAACGTGAACCAGCGTGAAATCCCGGAAGAGATTAAAGAC
CGCGCAACCACCCTGTACGAAATTACCGGTAAAGACTGGGATCGT
AAGGAGGTTTTGTTGAAGGTGCTCAAGCGCATTAGCGAGAACCTG
AAGAAATTCAAAGAGAAAGAAAATCTATATTTTCAAGGAGGAGGT
GGCAGTGGCGGCGGCAGTTCATTTAAAGAATTCAAGGGCAAGATC
GAAAGCAAAATGCTGTACCTGGGCGAGGAAGTGAAACTGCTGGGT
GAGGGCAAGATCACGGGTAAGTTGGTTGGTCTCTCTGAAAAAGGC
GGTGCGCTGATTTTGACCGAAGAGGGTATTAAAGAGATCTTATCC
GGCGAGTTCAGCCTGCGCCGTAGCGGTGGTTCG
SpyCatcher-ultraID-TEV site-GGGSx3-Cterm
(polypeptide)
(SEQ ID NO: 36)
MHHHHHHMVTTLSGLSGEQGPSGDMTTEEDSATHIKFSKRDEDGR
ELAGATMELRDSSGKTISTWISDGHVKDFYLYPGKYTFVETAAPD
GYEVATPIEFTVNEDGQVTVDGEATEGDAHTGSSGSGGGSGGGSG
GGSMFKNLIWLKEVDSTQERLKEWNVSYGTALVADRQTKGRGGPG
RKWLSQEGGLYFSFLLNPKEFENLLQLPLVLGLSVSEALEEITEI
PFSLKWPNDVYFQEKKVSGVLCELSKDKLIVGIGINVNQREIPEE
IKDRATTLYEITGKDWDRKEVLLKVLKRISENLKKFKEKENLYFQ
GGGGSGGGSGGGSSFKEFKGKIESKMLYLGEEVKLLGEGKITGKL
VGLSEKGGALILTEEGIKEILSGEFSLRRSGGS
SpyCatcher-ultraID-TEV site-GGGSx3-Cterm
(polynucleotide)
(SEQ ID NO: 37)
ATGGTAACTACACTATCAGGATTATCTGGCGAGCAGGGTCCGAGC
GGCGATATGACCACTGAGGAAGACTCTGCCACGCATATCAAATTT
TCGAAGCGCGACGAAGACGGTCGTGAACTGGCGGGTGCTACGATG
GAATTGCGTGATAGCAGCGGTAAGACCATTTCCACCTGGATTAGC
GACGGCCACGTGAAAGACTTCTACCTGTATCCAGGTAAATATACC
TTTGTTGAAACCGCGGCACCGGATGGCTACGAGGTGGCGACTCCG
ATCGAGTTCACGGTTAACGAAGATGGTCAAGTAACAGTAGATGGT
GAAGCTACAGAAGGTGATGCACATACAGGTTCAAGTGGAAGTGGA
GGTGGCAGTGGCGGCGGCAGTGGTGGAGGCAGTATGTTTAAGAAT
CTAATATGGTTAAAAGAAGTGGACAGCACCCAAGAACGTCTGAAG
GAGTGGAACGTTAGCTATGGTACTGCTCTGGTTGCGGATCGCCAG
ACCAAAGGTCGTGGTGGCCCGGGCCGTAAGTGGCTGTCGCAAGAA
GGTGGCCTCTACTTCAGCTTTCTGTTAAATCCGAAAGAGTTTGAA
AACTTGCTGCAGCTGCCGCTGGTCTTGGGTTTGTCCGTGTCTGAG
GCGCTGGAAGAGATCACGGAAATCCCGTTTTCTCTGAAGTGGCCA
AATGATGTTTATTTCCAAGAGAAGAAGGTCAGCGGTGTTCTTTGC
GAACTGTCCAAAGACAAACTGATCGTGGGCATCGGCATTAACGTG
AACCAGCGTGAAATCCCGGAAGAGATTAAAGACCGCGCAACCACC
CTGTACGAAATTACCGGTAAAGACTGGGATCGTAAGGAGGTTTTG
TTGAAGGTGCTCAAGCGCATTAGCGAGAACCTGAAGAAATTCAAA
GAGAAAGAAAATCTATATTTTCAAGGAGGAGGTGGCAGTGGCGGC
GGCAGTGGTGGAGGCAGTTCATTTAAAGAATTCAAGGGCAAGATC
GAAAGCAAAATGCTGTACCTGGGCGAGGAAGTGAAACTGCTGGGT
GAGGGCAAGATCACGGGTAAGTTGGTTGGTCTCTCTGAAAAAGGC
GGTGCGCTGATTTTGACCGAAGAGGGTATTAAAGAGATCTTATCC
GGCGAGTTCAGCCTGCGCCGTAGCGGTGGTTCG

In some embodiments, the disclosure provides a recombinant or synthetic polypeptide comprising a biotin protein ligase as defined above, i.e., a recombinant or synthetic polypeptide comprising a polypeptide fused to a biotin protein ligase or of the disclosure. The recombinant or synthetic polypeptide optionally comprises a spacer as defined above.

In some embodiments the polypeptide fused to a biotin protein ligase of the disclosure is an antibody, nanobody, or antigen binding fragment thereof. Some exemplary sequences are:

anti-mCD47 nanobody-ultraID
(murine CD47) (polypeptide)
(SEQ ID NO: 38)
QVQLVESGGGLVEPGGSLRLSCAASGIIFKINDMGWYRQAPGKRR
EWVAASTGGDEAIYRDSVKDRFTISRDAKNSVFLQMNSLKPEDTA
VYYCTAVISTDRDGTEWRRYWGQGTQVTVSSGGLPETGGGGGSGG
GSGGGSMFKNLIWLKEVDSTQERLKEWNVSYGTALVADRQTKGRG
GPGRKWLSQEGGLYFSFLLNPKEFENLLQLPLVLGLSVSEALEEI
TEIPFSLKWPNDVYFQEKKVSGVLCELSKDKLIVGIGINVNQREI
PEEIKDRATTLYEITGKDWDRKEVLLKVLKRISENLKKFKEK
anti-mCD47 nanobody-ultraID
(murine CD47) (polynucleotide)
(SEQ ID NO: 39)
CAAGTTCAGCTAGTAGAAAGTGGTGGAGGGTTGGTAGAGCCGGGT
GGCAGCCTGCGTCTGAGCTGTGCTGCGTCTGGCATTATCTTTAAA
ATCAATGATATGGGTTGGTATCGCCAAGCACCAGGTAAGCGTAGA
GAATGGGTTGCAGCTTCCACCGGCGGTGATGAGGCGATTTACCGC
GACAGCGTGAAGGACCGCTTCACCATTAGCCGTGACGCCAAGAAC
TCCGTGTTCTTGCAAATGAACAGCCTGAAACCGGAGGACACCGCG
GTGTACTACTGCACTGCGGTCATCTCGACCGATCGTGATGGTACG
GAATGGCGTCGTTATTGGGGTCAGGGTACGCAGGTTACCGTTTCT
AGCGGCGGCCTGCCGGAAACCGGTGGCGGAGGTGGCAGTGGCGGC
GGCAGTGGTGGAGGCAGTATGTTTAAGAATCTAATATGGTTAAAA
GAAGTGGACAGCACCCAAGAACGTCTGAAGGAGTGGAACGTTAGC
TATGGTACTGCTCTGGTTGCGGATCGCCAGACCAAAGGTCGTGGT
GGCCCGGGCCGTAAGTGGCTGTCGCAAGAAGGTGGCCTCTACTTC
AGCTTTCTGTTAAATCCGAAAGAGTTTGAAAACTTGCTGCAGCTG
CCGCTGGTCTTGGGTTTGTCCGTGTCTGAGGCGCTGGAAGAGATC
ACGGAAATCCCGTTTTCTCTGAAGTGGCCAAATGATGTTTATTTC
CAAGAGAAGAAGGTCAGCGGTGTTCTTTGCGAACTGTCCAAAGAC
AAACTGATCGTGGGCATCGGCATTAACGTGAACCAGCGTGAAATC
CCGGAAGAGATTAAAGACCGCGCAACCACCCTGTACGAAATTACC
GGTAAAGACTGGGATCGTAAGGAGGTTTTGTTGAAGGTGCTCAAG
CGCATTAGCGAGAACCTGAAGAAATTCAAAGAGAAA
anti-hCD47 nanobody-ultraID
(human CD47) (polypeptide)
(SEQ ID NO: 40)
QVQLQESGGGSVQAGGSLRLSCAASGYTYSSYCMGWFRQAPGKER
EGVAAIYPDAGITFYTDSVKGRFTISRDNAKNTLFLQMNSLKPED
TATYYCAAAPPSVPCRLVVARYNYWGQGTQVTVSSGGGSGGGSGG
GSMFKNLIWLKEVDSTQERLKEWNVSYGTALVADRQTKGRGGPGR
KWLSQEGGLYFSFLLNPKEFENLLQLPLVLGLSVSEALEEITEIP
FSLKWPNDVYFQEKKVSGVLCELSKDKLIVGIGINVNQREIPEEI
KDRATTLYEITGKDWDRKEVLLKVLKRISENLKKFKEK
anti-hCD47 nanobody-ultraID
(human CD47) (polynucleotide)
(SEQ ID NO: 41)
CAAGTACAGCTACAAGAATCAGGTGGAGGGTCGGTCCAGGCGGGC
GGTTCTCTGCGTCTGAGCTGCGCGGCGTCCGGTTATACGTACAGC
TCCTATTGCATGGGTTGGTTCCGCCAGGCTCCGGGTAAAGAGCGC
GAGGGCGTGGCTGCCATCTACCCGGACGCGGGCATCACCTTTTAT
ACTGACAGCGTGAAGGGCCGTTTCACCATTAGCCGTGATAATGCA
AAAAACACCCTGTTTCTGCAAATGAACAGCTTGAAGCCGGAAGAT
ACCGCAACCTACTACTGCGCGGCTGCGCCACCGTCCGTTCCGTGT
AGATTGGTTGTGGCCCGTTATAACTACTGGGGTCAAGGTACGCAG
GTTACCGTAAGCTCTGGAGGTGGCAGTGGCGGCGGCAGTGGTGGA
GGCAGTATGTTTAAGAATCTAATATGGTTAAAAGAAGTGGACAGC
ACCCAAGAACGTCTGAAGGAGTGGAACGTTAGCTATGGTACTGCT
CTGGTTGCGGATCGCCAGACCAAAGGTCGTGGTGGCCCGGGCCGT
AAGTGGCTGTCGCAAGAAGGTGGCCTCTACTTCAGCTTTCTGTTA
AATCCGAAAGAGTTTGAAAACTTGCTGCAGCTGCCGCTGGTCTTG
GGTTTGTCCGTGTCTGAGGCGCTGGAAGAGATCACGGAAATCCCG
TTTTCTCTGAAGTGGCCAAATGATGTTTATTTCCAAGAGAAGAAG
GTCAGCGGTGTTCTTTGCGAACTGTCCAAAGACAAACTGATCGTG
GGCATCGGCATTAACGTGAACCAGCGTGAAATCCCGGAAGAGATT
AAAGACCGCGCAACCACCCTGTACGAAATTACCGGTAAAGACTGG
GATCGTAAGGAGGTTTTGTTGAAGGTGCTCAAGCGCATTAGCGAG
AACCTGAAGAAATTCAAAGAGAAA

The recombinant or synthetic polypeptide of the disclosure may also comprise purification moieties or tags to facilitate their purification (e.g., prior to use in the methods and uses of the disclosure discussed below). Any suitable purification moiety or tag may be incorporated into the polypeptide and such moieties are well known in the art. For instance, in some embodiments, the recombinant or synthetic polypeptide may comprise a peptide purification tag or moiety, e.g., a His-tag sequence. Such purification moieties or tags may be incorporated at any position within the polypeptide. In some preferred embodiments, the purification moiety is located at or towards (i.e., within 5, 10, 15, 20 amino acids of) the N- or C-terminus of the polypeptide.

As noted above, an advantage of the present disclosure arises from the fact that the biotin protein ligases incorporated in fusion proteins (e.g., the recombinant or synthetic polypeptides of the disclosure) may be completely genetically encoded. Thus, in a further aspect, the disclosure provides a nucleic acid molecule encoding a biotin protein ligase or recombinant or synthetic polypeptide as defined above.

In an embodiment, a biotin protein ligase is encoded by one of the polynucleotide sequences described herein or by a polynucleotide sequence having at least 85% identity to such sequence. Nucleic acid sequence identity may be determined by, e.g., FASTA Search using GCG packages, with default values and a variable pamfactor, and gap creation penalty set at 12.0 and gap extension penalty set at 4.0 with a window of 6 nucleotides. Preferably said comparison is made over the full length of the sequence, but may be made over a smaller window of comparison, e.g., less than 600, 500, 400, 300, 200, 100 or 50 contiguous nucleotides.

The nucleic acid molecules of the disclosure may be made up of ribonucleotides and/or deoxyribonucleotides as well as synthetic residues, e.g. synthetic nucleotides, that are capable of participating in Watson-Crick type or analogous base pair interactions. Preferably, the nucleic acid molecule is DNA or RNA.

The nucleic acid molecules described above may be operatively linked to an expression control sequence, or a recombinant DNA cloning vehicle or vector containing such a recombinant DNA molecule. This allows intracellular expression of the peptides and polypeptides of the disclosure as a gene product, the expression of which is directed by the gene(s) introduced into cells of interest. Gene expression is directed from a promoter active in the cells of interest and may be inserted in any form of linear or circular nucleic acid (e.g., DNA) vector for incorporation in the genome or for independent replication or transient transfection/expression. Suitable transformation or transfection techniques are well described in the literature. Alternatively, the naked nucleic acid (e.g., DNA or RNA, which may include one or more synthetic residues, e.g., base analogues) molecule may be introduced directly into the cell for the production of peptides and polypeptides of the disclosure. Alternatively, the nucleic acid may be converted to mRNA by in vitro transcription and the relevant proteins may be generated by in vitro translation.

Appropriate expression vectors include appropriate control sequences such as for example translational (e.g., start and stop codons, ribosomal binding sites) and transcriptional control elements (e.g., promoter-operator regions, termination stop sequences) linked in matching reading frame with the nucleic acid molecules of the disclosure. Appropriate vectors may include plasmids and viruses (including both bacteriophage and eukaryotic viruses). Suitable viral vectors include baculovirus and also adenovirus, adeno-associated virus, herpes and vaccinia/pox viruses. Many other viral vectors are described in the art.

Examples of suitable vectors include bacterial and mammalian expression vectors pGEX-KG, pEF-neo and pEF-HA.

As noted above, the recombinant or synthetic polypeptide of the disclosure may comprise additional sequences (e.g. peptide/polypeptides tags to facilitate purification of the polypeptide) and thus the nucleic acid molecule may conveniently be fused with DNA encoding an additional peptide or polypeptide, e.g. His-tag, maltose-binding protein, to produce a fusion protein on expression.

Thus, viewed from a further aspect, the present disclosure provides a vector, preferably an expression vector, comprising a nucleic acid molecule as defined above. Other aspects of the disclosure include methods for preparing recombinant nucleic acid molecules according to the disclosure, comprising inserting a nucleic acid molecule of the disclosure encoding the biotin protein ligase and/or polypeptide of the disclosure into vector nucleic acid.

Nucleic acid molecules of the disclosure, preferably contained in a vector, may be introduced into a cell by any appropriate means. Suitable transformation or transfection techniques are well described in the literature. Numerous techniques are known and may be used to introduce such vectors into prokaryotic or eukaryotic cells for expression. Preferred host cells for this purpose include insect cell lines, yeast, mammalian cell lines or E. coli, such as strain BL21/DE3. The disclosure also extends to transformed or transfected prokaryotic or eukaryotic host cells containing a nucleic acid molecule, particularly a vector as defined above.

Thus, in another aspect, there is provided a recombinant host cell containing a nucleic acid molecule and/or vector as described above. By “recombinant” is meant that the nucleic acid molecule and/or vector has been introduced into the host cell. The host cell may or may not naturally contain an endogenous copy of the nucleic acid molecule, but it is recombinant in that an exogenous or further endogenous copy of the nucleic acid molecule and/or vector has been introduced.

A further aspect of the disclosure provides a method of preparing a biotin protein ligase and/or fusion polypeptide of the disclosure, which comprises culturing a host cell containing a nucleic acid molecule as defined above, under conditions whereby said nucleic acid molecule encoding said biotin protein ligase and/or polypeptide is expressed and recovering said molecule (biotin protein ligase and/or polypeptide) thus produced. The expressed biotin protein ligase and/or polypeptide forms a further aspect of the disclosure.

In some embodiments, the biotin protein ligase s and/or polypeptides of the disclosure, or for use in the method and uses of the disclosure, may be generated synthetically, e.g., by ligation of amino acids or smaller synthetically generated peptides, or more conveniently by recombinant expression of a nucleic acid molecule encoding said polypeptide as described hereinbefore.

Nucleic acid molecules of the disclosure may be generated synthetically by any suitable means known in the art.

Thus, the biotin protein ligase and/or fusion polypeptide of the disclosure may be an isolated, purified, recombinant or synthesized biotin protein ligase or polypeptide. The term “polypeptide” is used herein interchangeably with the term “protein”. As noted above, the term polypeptide typically includes any amino acid sequence comprising at least 40 consecutive amino acid residues, e.g., at least 50, 60, 70, 80, 90, 100, 150 amino acids, such as 40-1000, 50-900, 60-800, 70-700, 80-600, 90-500, 100-400 amino acids.

Standard amino acid nomenclature is used herein. Thus, the full name of an amino acid residue may be used interchangeably with one letter code or three letter abbreviations. For instance, lysine may be substituted with K or Lys, isoleucine may be substituted with I or Ile, and so on. Moreover, the terms aspartate and aspartic acid, and glutamate and glutamic acid are used interchangeably herein and may be replaced with Asp or D, or Glu or E, respectively.

While the biotin protein ligases and fusion polypeptides comprising such ligases may be produced recombinantly, it will be evident that the biotin protein ligases of the disclosure may be conjugated to proteins or other entities, e.g. molecules, as defined above by other means. In other words, the biotin protein ligase and another agent, such as a protein, may be produced separately by any suitable means, e.g., recombinantly, and subsequently conjugated (joined) to form a biotin protein ligase-other component conjugate that can be used in the methods and uses of the disclosure. For instance, the biotin protein ligases of the disclosure may be produced synthetically or recombinantly, as described above, and conjugated to another component, e.g., a protein via a non-peptide linker or spacer, e.g., a chemical linker or spacer.

Thus, in some embodiments, the biotin protein ligase and other component, e.g., protein, may be joined together either directly through a bond or indirectly through a linking group. Where linking groups are employed, such groups may be chosen to provide for covalent attachment of the biotin protein ligase and other entity, e.g. protein, through the linking group. Linking groups of interest may vary widely depending on the nature of the other entity, e.g. protein. The linking group, when present, is in many embodiments biologically inert.

A variety of linking groups are known to those of skill in the art and find use in the disclosure. In representative embodiments, the linking group is generally at least about 50 daltons, usually at least about 100 daltons and may be as large as 1000 daltons or larger, for example up to 1000000 daltons if the linking group contains a spacer, but generally will not exceed about 500 daltons and usually will not exceed about 300 daltons. Generally, such linkers will comprise a spacer group terminated at either end with a reactive functionality capable of covalently bonding to the biotin protein ligase and other molecule or component, e.g. protein.

Spacer groups of interest may include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine, oligoethylene glycol and polyethylene glycol. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the subject blocking reagent include heterofunctional compounds, such as azidobenzoyl hydrazide, N-[4-(p-azidosalicylamino)butyl]-3′-[2′-pyridyldithio] propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl-4-[N-maleimidomethyl]cyclohexane-1-carboxylate, 3-(2-pyridyldithio) propionic acid N-hydroxysuccinimide ester (SPDP), 4-(N-maleimidomethyl)-cyclohexane-1-carboxylic acid N-hydroxysuccinimide ester (SMCC), and the like. For instance, a spacer may be formed with an azide reacting with alkyne or a tetrazine reacting with trans-cyclooctene or norbornene. In other embodiments, the linker group can be a non-natural amino acid with an extended backbone (e.g., aminohexanoic acid).

In some embodiments, it may be useful to modify one or more residues in the biotin protein ligase and/or polypeptide to facilitate the conjugation of these molecules and/or to improve the stability of the biotin protein ligase and/or fusion polypeptide. Thus, in some embodiments, the biotin protein ligase or fusion polypeptide of, or for use in, the disclosure may comprise unnatural or non-standard amino acids.

In some embodiments, the biotin protein ligase or polypeptide of, or for use in, the disclosure may comprise one or more, e.g., at least 1, 2, 3, 4, 5 non-conventional amino acids, such as 10, 15, 20 or more non-conventional, i.e., amino acids which possess a side chain that is not coded for by the standard genetic code, termed herein “non-coded amino acids” (see e.g. Table 1). These may be selected from amino acids which are formed through metabolic processes such as ornithine or taurine, and/or artificially modified amino acids such as 9H-fluoren-9-ylmethoxycarbonyl (Fmoc), (tert)-(B)utyl (o)xy (c)arbonyl (Boc), 2,2,5,7,8-pentamethylchroman-6-sulphonyl (Pmc) protected amino acids, or amino acids having the benzyloxy-carbonyl (Z) group.

Examples of non-standard or structural analogue amino acids which may be used in the peptide linkers or polypeptides of, and for use in, the disclosure are D amino acids, amide isosteres (such as N-methyl amide, retro-inverse amide, thioamide, thioester, phosphonate, ketomethylene, hydroxymethylene, fluorovinyl, (E)-vinyl, methyleneamino, methylenethio or alkane), L-N methylamino acids, D-.alpha. methylamino acids, D-N-methylamino acids.

### Ligands Conjugated to Biotin Protein Ligases

Ligands useful in the methods of the invention include, but are not limited to nanobodies, antibodies, antigen-binding fragments thereof, and aptamers. In particular embodiments, ligands useful in the methods described herein include those that bind to markers on the cell surface. Such markers include, but are not limited to, markers specific to particular cell types, such as immune cells, tumor cells, cells at a particular developmental stage, as well as cells defined by a particular disease state (cancer, autoimmune disease).

Ligands of the invention can recognize a wide variety of tissue types, including, but not limited to, breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain, as well as tumor cells derived from such tissues.

Exemplary tumor markers expressed by a wide range of tumor cells include CTLA4, PD1, EpCAM, CD47, CD44, and CEA. Using standard methods, tumor-specific ligands (e.g., antibodies, antigen binding fragments, nanobodies, aptamers) can be selected that bind virtually any tumor marker known in the art. Markers to which tumor-specific ligands bind are also well known in the art. For example, markers bound by the tumor-specific aptamers of the invention include, but are not limited to, those known in the art to be present on CA-125, gangliosides G (D2), G (M2) and G (D3), CD20, CD52, CD33, Ep-CAM, CEA, bombesin-like peptides, prostate specific antigen (PSA), prostate-specific membrane antigen (PSMA), HER2/neu, epidermal growth factor receptor, erbB2, erbB3, erbB4, CD44v6, Ki-67, VEGF, VEGFRs (e.g., VEGFR3), estrogen receptors, Lewis-Y antigen, TGFβ1, IGF-1 receptor, EGFα, c-Kit receptor, transferrin receptor, IL-2R, CO17-1A, Pd-1, CTLa4, tumor-associated antigen MUC1, TGF beta receptor, and TGF beta.

### Polynucleotides

The compositions and methods described herein in various embodiments include an isolated polynucleotide sequence or an isolated polynucleotide molecule that encodes a modified biotin ligase or a biotin ligase fusion protein. Accordingly, in some embodiments, the isolated polynucleotide sequence or isolated polynucleotide molecule comprises or consists of a polynucleotide sequence that encodes a polypeptide molecule of a modified biotin ligase or a biotin ligase fusion protein, or a functional portion thereof, as described herein. In an embodiment, a composition comprises a combination of the isolated polynucleotide sequences or isolated polynucleotide molecules as described herein.

Any of a variety of expression vectors (prokaryotic or eukaryotic) known to and used by those of ordinary skill in the art may be employed to express recombinant polypeptides described herein. Expression can be achieved in any appropriate host cell that has been transformed or transfected with an expression vector containing a polynucleotide (DNA) molecule that encodes a recombinant polypeptide. Suitable host cells include prokaryotes, yeast and higher eukaryotic cells. By way of example, the host cells employed include, without limitation, E. coli, yeast, insect cells, or a mammalian cell line such as COS or CHO. The DNA sequences expressed in this manner can encode any of the polypeptides described herein, including variants thereof.

Uses of plasmids, vectors or viruses (viral vectors) containing polynucleotides encoding the modified biotin ligase or a biotin ligase fusion protein as described herein include generation of mRNA or protein in vitro or in vivo. In related embodiments, host cells transformed with the plasmids, vectors, or virus vectors are provided, as described above. Nucleic acid molecules can be inserted into a construct (such as a prokaryotic expression plasmid, a eukaryotic expression vector, or a viral vector construct, which can, optionally, replicate and/or integrate into a recombinant host cell by known methods. The host cell can be a eukaryote or prokaryote and can include, for example and without limitation, yeast (such as Pichia pastoris or Saccharomyces cerevisiae), bacteria (such as E. coli, or Bacillus subtilis), animal cells or tissue (CHO or COS cells), insect Sf9 cells (such as baculoviruses infected SF9 cells), or mammalian cells (somatic or embryonic cells, Human Embryonic Kidney (HEK) cells, Chinese hamster ovary (CHO) cells, HeLa cells, human 293 cells (Expi293F), and monkey COS-7 cells). Suitable host cells also include a mammalian cell, a bacterial cell, a yeast cell, an insect cell, a plant cell, or an algal cell.

### Pharmaceutical Formulations

Biotin protein ligases of the disclosure and fusion proteins comprising such biotin protein ligases fused to a targeting agent (e.g., an antibody, nanobody or antigen fragment thereof) may be administered systemically, for example, formulated in a pharmaceutically-acceptable buffer, such as physiological saline. Preferable routes of administration include, for example, subcutaneous, intravenous, interperitoneal, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the disease (e.g., neoplasia, autoimmune disorder). Generally, amounts will be in the range of those used for other agents used in the treatment of other diseases, including those associated with neoplasia or autoimmune disorders), although in certain instances lower amounts will be needed because of the increased specificity of the compound.

### Formulation of Pharmaceutical Compositions

Pharmaceutical compositions comprising a biotin protein ligase (e.g. a fusion protein comprising a biotin protein ligase and a targeting agent, and a cargo agent attached to a biotin binding agent (e.g., anti-biotin antibody, or antigen-binding fragment thereof, streptavidin, avidin, avidin variant) may be contained in any appropriate amount in any suitable carrier substance, and such proteins are generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to some aspects and embodiments herein may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the imaging agent and/or therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a disease (e.g. neoplasia, autoimmune disorder), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to some aspects and embodiments herein may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

The following examples are put forth so as to provide those of ordinary skill in the art with a complete disclosure and description of how to make and use the assay, screening, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

### Example 1: Expression, Isolation, and In Vitro Activity of an E. coli Biotin Ligase

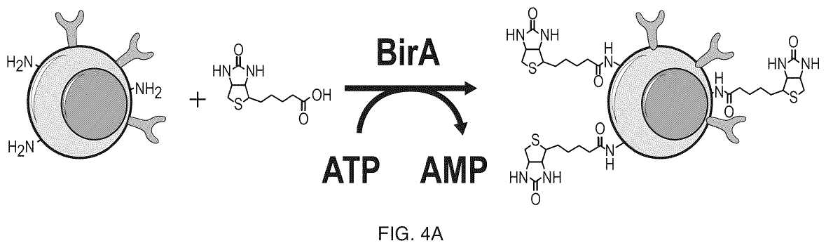

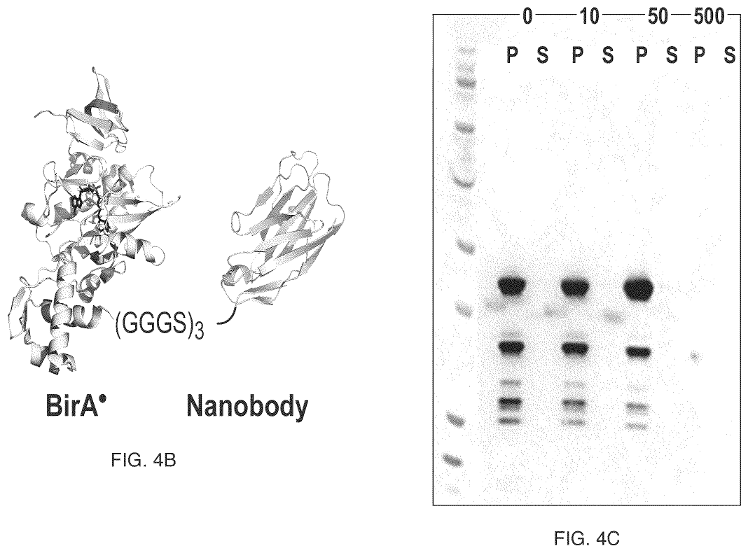

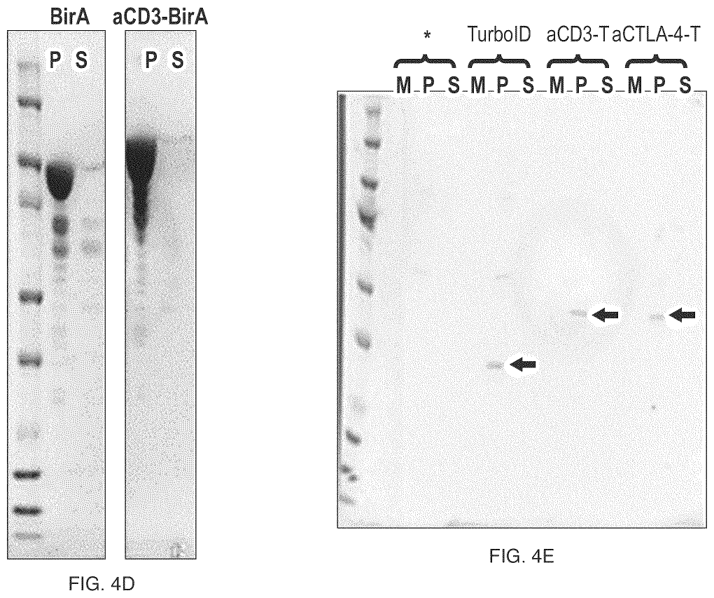

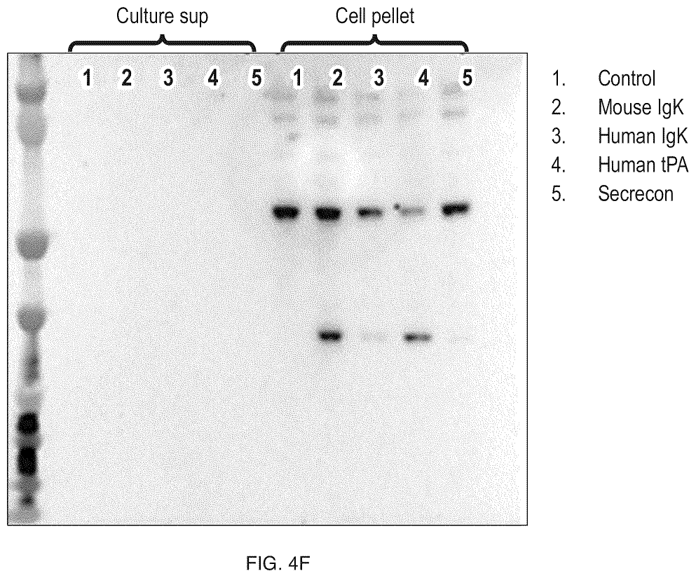

BirA, a 33 kDa biotin ligase from E. coli, functions in generating the holo form of the carboxylase that converts acetyl-CoA to malonyl-CoA and plays a crucial role in lipid metabolism (specifically, biotin-carboxyl carrier protein which is a subunit of acetyl-CoA carboxylase). The structure of BirA and its general reaction scheme are shown in FIGS. 4A-4B. A single active site mutation, R118G, eliminates specificity for the substrate target sequence and instead causes the activated biotinyl-AMP species to be released, upon which it reacts with the primary amine of any proximal lysine residues. This variant is capable of biotinylating any lysine within a ˜20 nm radius and was employed in protein proximity labeling. This enzyme was ideal for the targeting system, meeting all of the desired criteria: 1) reactive with a substrate natively present on the surface of any cell; 2) capable of installing a cargo of interest and/or a recognition ‘handle’; 3) well characterized structure and activity; 4) engineered for optimal activity, split variant(s); and 5) overall simplicity of the corresponding system—i.e. a small, easy-to-handle enzyme with substrates that are present or readily supplied in locations of interest like the tumor microenvironment.

An E. coli BirA* single mutant variant (R118G, FIG. 4C) and a multi-mutant optimized for high activity (FIG. 4D) were expressed in E. coli across a range of conditions: inducer concentration (0-500 μM), expression temperature (37, 30, and 18° C.), fusion with a chaperone (NusA and mRID), and media with optimized controlled sugar release (EnPresso). Nanobody enzyme fusions (FIG. 4D) were also expressed. Multi-mutant BirA* was isolated and tested in a biotinylation assay. Mammalian expression yielded a functional product (FIGS. 4E-4F).

### Example 2: Expression, Isolation, and In Vitro Activity of an A. Aeolicus Biotin Ligase

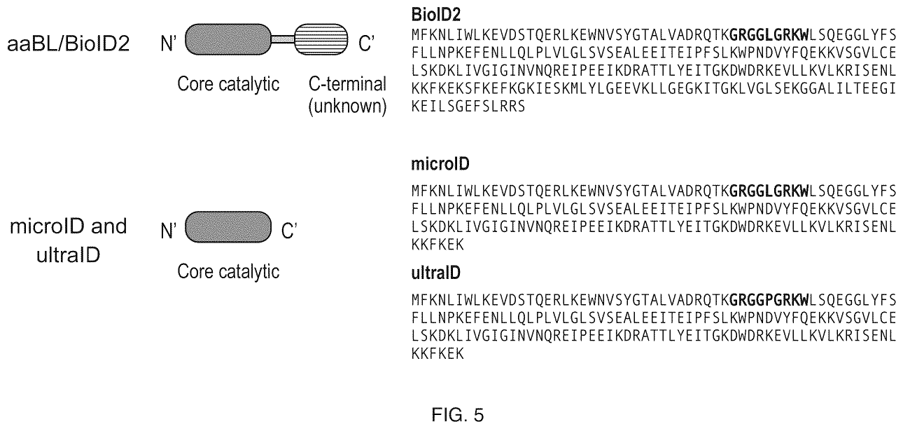

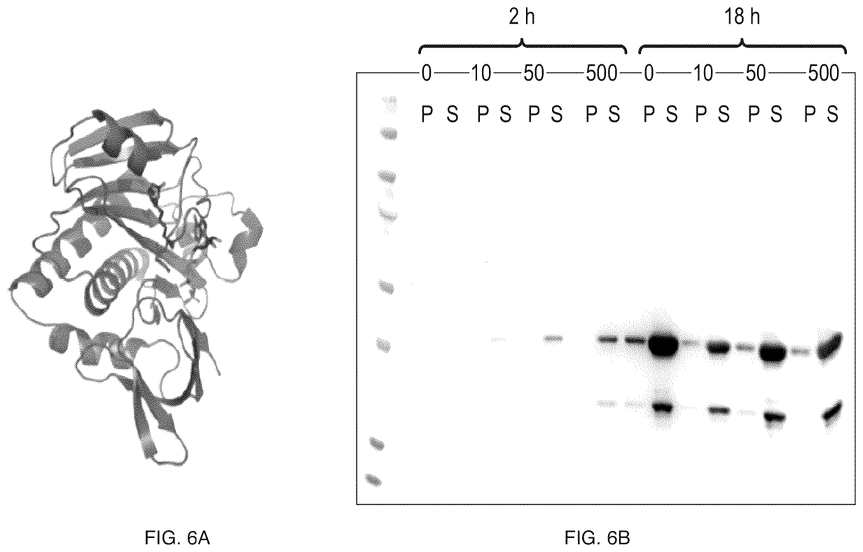

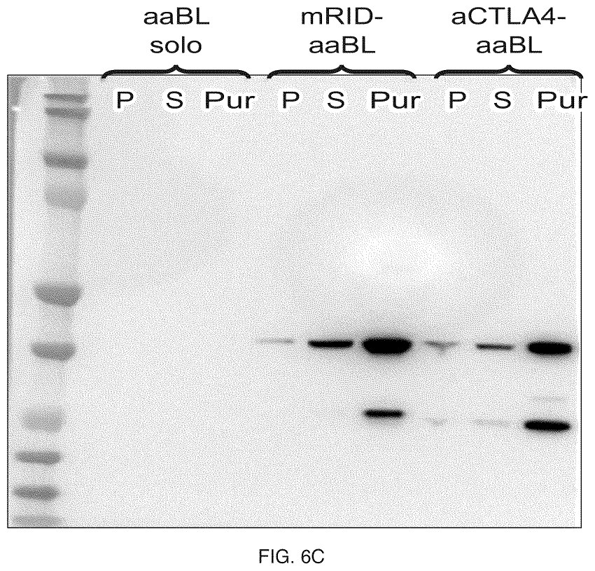

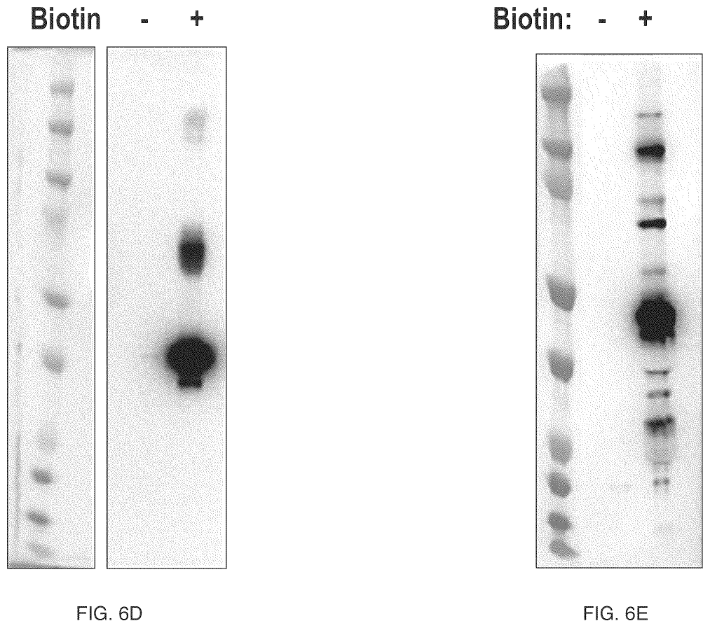

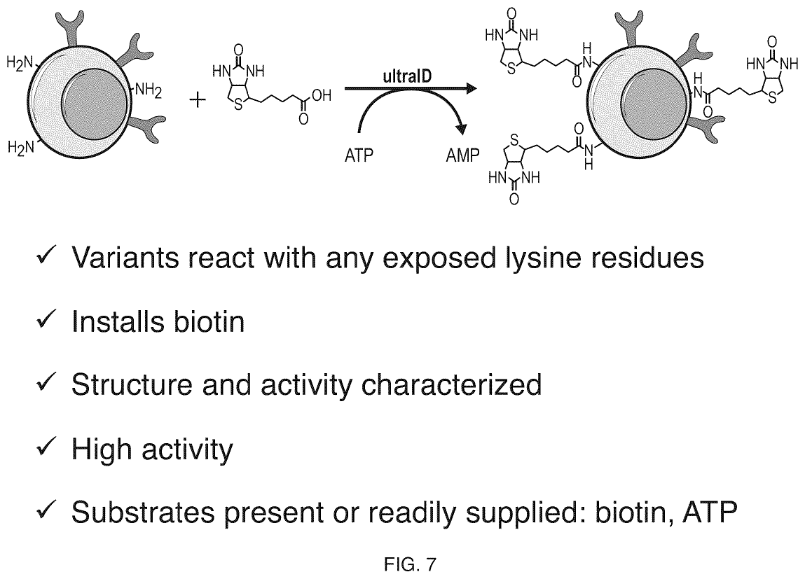

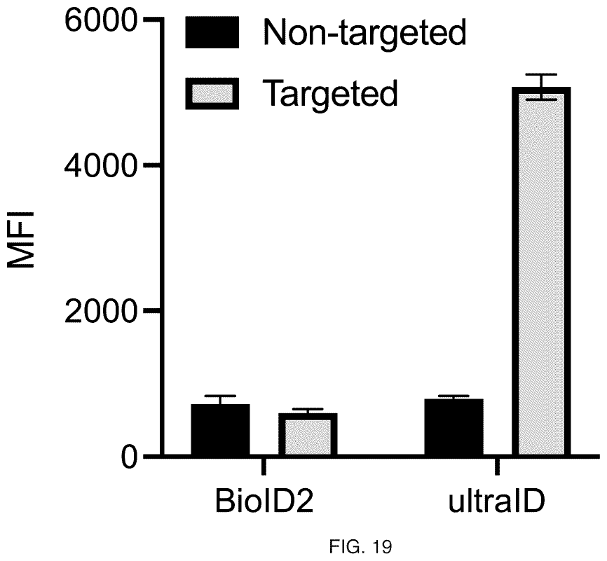

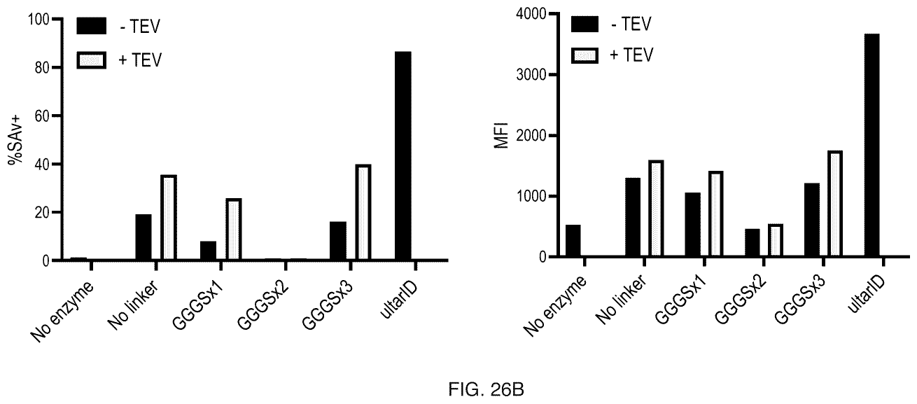

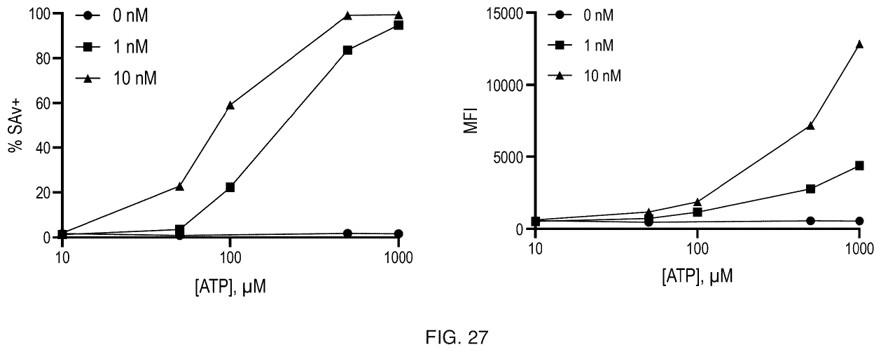

The A. aeolicus biotin ligase (aaBL) performs a similar function to E. coli BirA, biotinylating a particular sequence to down-regulate biotin synthesis. An aaBL variant with a similar active site mutation (R40G) was demonstrated to enable broad reactivity with proximal lysine residues. FIG. 5 shows that MicroID is a truncation of aaBL/BioID2 after K171, cleaving off the C-terminal domain. UltraID includes an additional active site mutation, L41P. The amino acid sequences for aaBL/BioID2, MicroID, and UltraID are listed. This small (˜20 kDa) enzyme represents the core catalytic domain of the original ligase, evolved for increased activity (FIG. 19). In addition, aaBL is ˜9 kDa and 100 amino acid residues smaller than BirA. This enzyme meets the same criteria listed above, with the exception of already developed split variants. Although this enzyme did not express well alone, including an N-terminal chaperone (mRID) or a targeting ligand (aCTLA-4 nanobody) allowed for robust expression across a range of inducer concentrations and expression conditions (FIGS. 6A-6C). These constructs were tested in a biotinylation assay, which confirmed their activity in vitro (FIGS. 6D-6E). FIG. 7 shows the labeling scheme for UltraID (A. aeolicus biotin ligase variant) of biotin onto a target cell using ATP. The biotin ligase variant reacts with any exposed lysine residue, covalently attaches biotin, has a structure and activity that are well characterized, has high activity, and uses endogenous substrates that are present or readily supplied (i.e., biotin and ATP). aCD4-ultraID remains active down to ˜50 μM ATP, although the activity drops substantially. Comparing activity across longer timescales will likely be more representative of an in vivo setting. The targeted enzyme was active at typical tumor micro-environment extracellular ATP concentrations (FIG. 27).

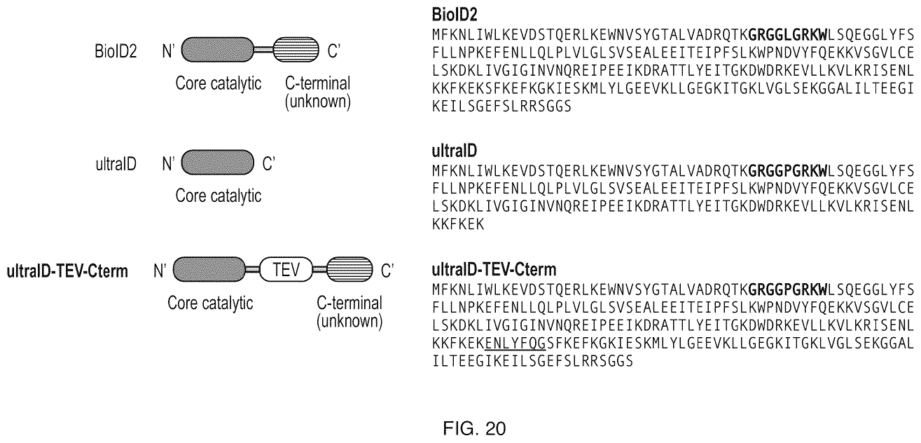

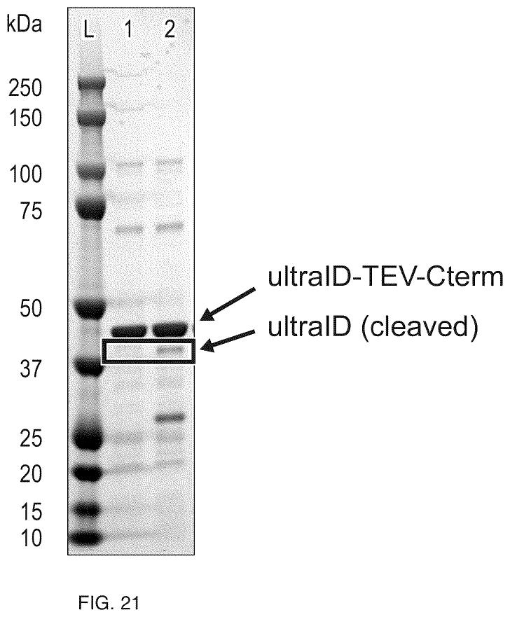

The UltraID enzyme was further modified by including a hindered variant, which includes a cleavable linker (FIG. 20). The hindered variant was designed to have less activity unless and/or until the C-terminal peptide is cleaved, increasing specificity of the system by increasing the activity (cleaving the C-terminus) only when bound to a specific cell type. FIG. 21 shows a stained gel with 1) ultraID-TEV site-Cterm and 2) ultraID-TEV site-Cterm+TEV protease. ultraID-TEV-Cterm was treated with TEV protease (NEB) overnight at 4° C. then analyzed by SDS-PAGE. A small amount of cleaved ultraID is detectable after TEV treatment, but cleavage appears to be relatively inefficient.

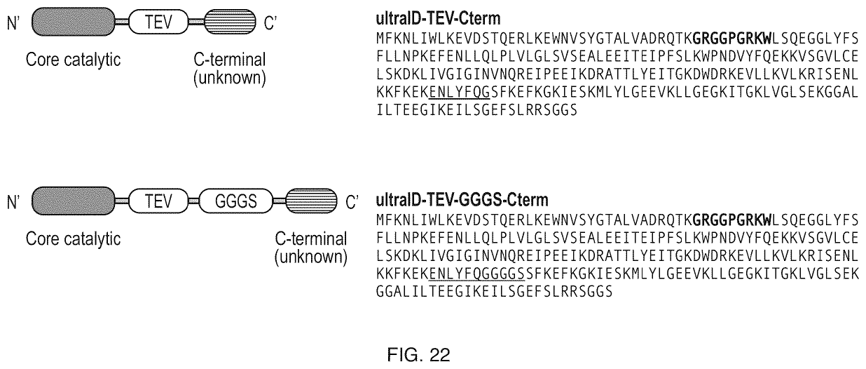

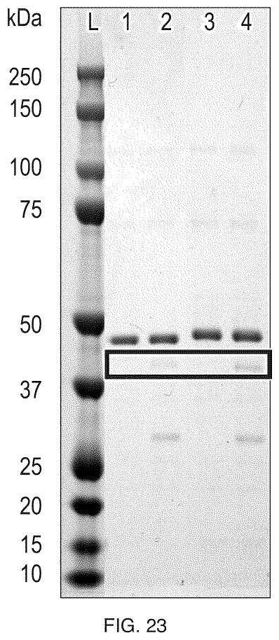

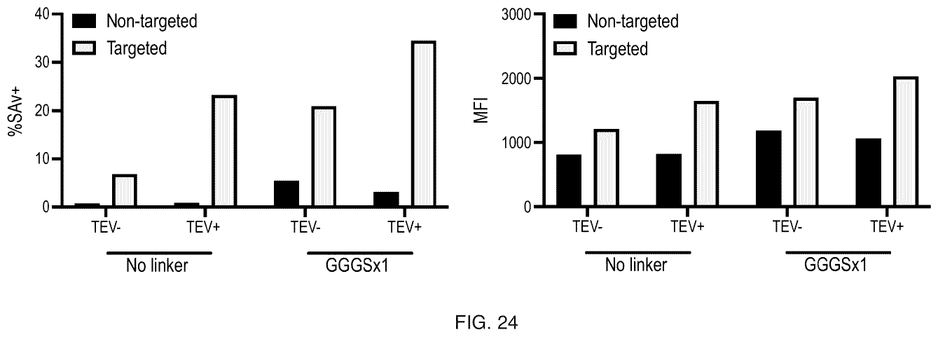

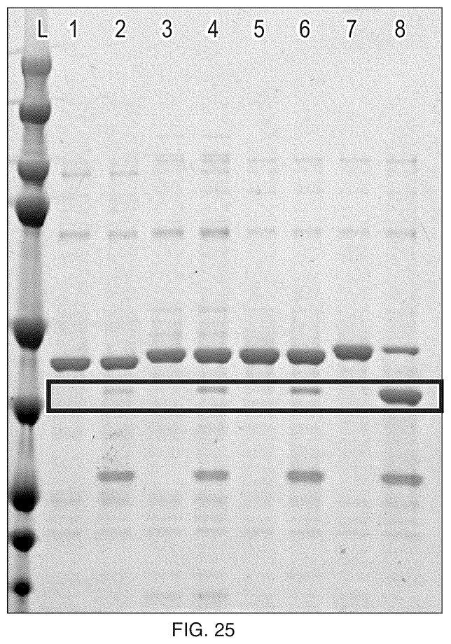

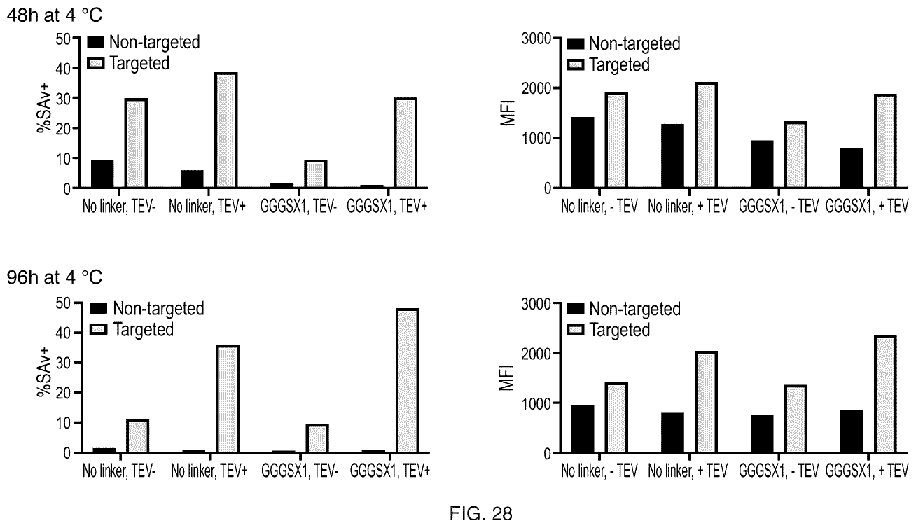

Incorporating a flexible GGGS spacer (SEQ ID NO: 4) after the TEV site introduces greater flexibility and allows the TEV protease greater access, improving cleavage efficiency (FIG. 22). Hindered ultraID constructs were treated with TEV protease overnight at 4° C. then analyzed by SDS-PAGE. The addition of a GGGS spacer (SEQ ID NO: 4) yielded more efficient proteolytic cleavage, but the hindered ultraID remained predominantly intact (FIG. 23). FIG. 24 shows that after pre-treating hindered ultraID variants with TEV protease (NEB) for 24 hours, the pre-cleaved constructs were conjugated to an aCD4 nanobody and used to treat U-937s. Some gain in biotin-labeling activity was observed with TEV treatment, indicating some release of the more active ultraID. Increased cell targeting after TEV treatment is shown in FIG. 28. Hindered ultraID constructs were treated with TEV protease (NEB) overnight at 4° C. then analyzed by SDS-PAGE. TEV cleavage efficiency increased with the addition of the (GGGS)ₙ spacer (SEQ ID NO: 4), with the most significant improvement observed with (GGGS)₃ (SEQ ID NO: 15). (FIG. 25).

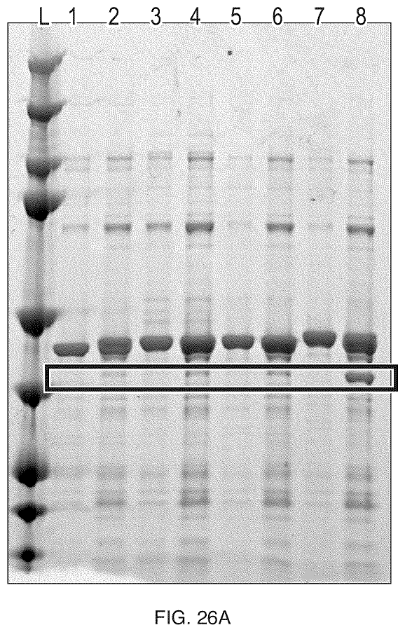

The UltraID enzyme was also fused with the SpyCatcher domain, where a modular version of this system was generated that can be readily combined with any targeting ligand fused to Spy Tag, a small (16-mer) peptide. Using molecular biology or bioconjugation chemistry, this format allowed attachment of a wide range of targeting ligands to this optimized biotin ligase. Hindered ultraID constructs were treated with recombinantly expressed SpyCatcher-TEV protease overnight at 4° C. then analyzed by SDS-PAGE (FIG. 26). SpyCatcher-TEV was active, with the most efficient cleavage observed for the (GGGS)₃ linker (SEQ ID NO: 15) variant.

SpyCatcher-ultra ID sequence:
(SEQ ID NO: 42)
MVTTLSGLSGEQGPSGDMTTEEDSATHIKFSKRDEDGRELAGATM
ELRDSSGKTISTWISDGHVKDFYLYPGKYTFVETAAPDGYEVATP
IEFTVNEDGQVTVDGEATEGDAHTGSSGSGGGSGGGSGGGSMFKN
LIWLKEVDSTQERLKEWNVSYGTALVADRQTKGRGGPGRKWLSQE
GGLYFSFLLNPKEFENLLQLPLVLGLSVSEALEEITEIPFSLKWP
NDVYFQEKKVSGVLCELSKDKLIVGIGINVNQREIPEEIKDRATT
LYEITGKDWDRKEVLLKVLKRISENLKKFKEKHHHHHH
SpyCatcher-ultra ID-TEV site-Cterm sequence:
(SEQ ID NO: 30)
MHHHHHHMVTTLSGLSGEQGPSGDMTTEEDSATHIKFSKRDEDGR
ELAGATMELRDSSGKTISTWISDGHVKDFYLYPGKYTFVETAAPD
GYEVATPIEFTVNEDGQVTVDGEATEGDAHTGSSGSGGGSGGGSG
GGSMFKNLIWLKEVDSTQERLKEWNVSYGTALVADRQTKGRGGPG
RKWLSQEGGLYFSFLLNPKEFENLLQLPLVLGLSVSEALEEITEI
PFSLKWPNDVYFQEKKVSGVLCELSKDKLIVGIGINVNQREIPEE
IKDRATTLYEITGKDWDRKEVLLKVLKRISENLKKFKEKENLYFQ
GSFKEFKGKIESKMLYLGEEVKLLGEGKITGKLVGLSEKGGALIL
TEEGIKEILSGEFSLRRSGGS
SpyCatcher-ultra ID-TEV site-GGGSx3-Cterm
sequence:
(SEQ ID NO: 36)
MHHHHHHMVTTLSGLSGEQGPSGDMTTEEDSATHIKFSKRDEDGR
ELAGATMELRDSSGKTISTWISDGHVKDFYLYPGKYTFVETAAPD
GYEVATPIEFTVNEDGQVTVDGEATEGDAHTGSSGSGGGSGGGSG
GGSMFKNLIWLKEVDSTQERLKEWNVSYGTALVADRQTKGRGGPG
RKWLSQEGGLYFSFLLNPKEFENLLQLPLVLGLSVSEALEEITEI
PFSLKWPNDVYFQEKKVSGVLCELSKDKLIVGIGINVNQREIPEE
IKDRATTLYEITGKDWDRKEVLLKVLKRISENLKKFKEKENLYFQ
GGGGSGGGSGGGSSFKEFKGKIESKMLYLGEEVKLLGEGKITGKL
VGLSEKGGALILTEEGIKEILSGEFSLRRSGGS

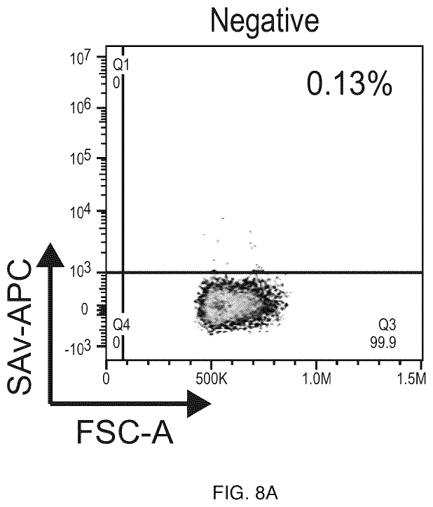

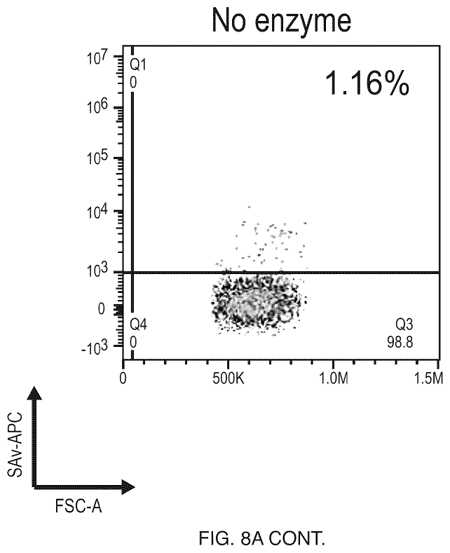

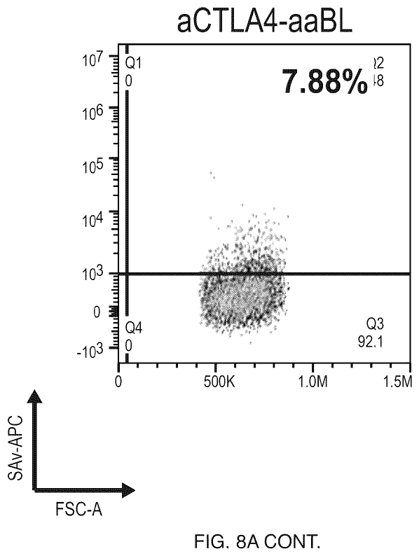

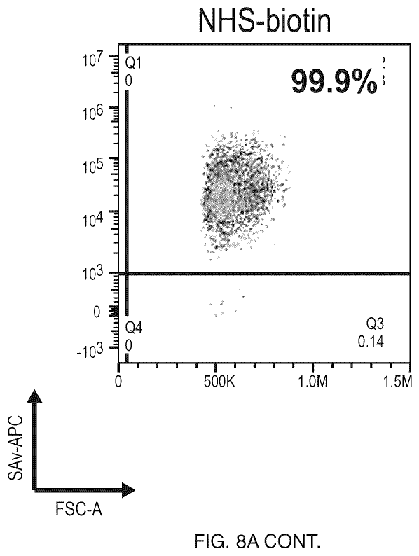

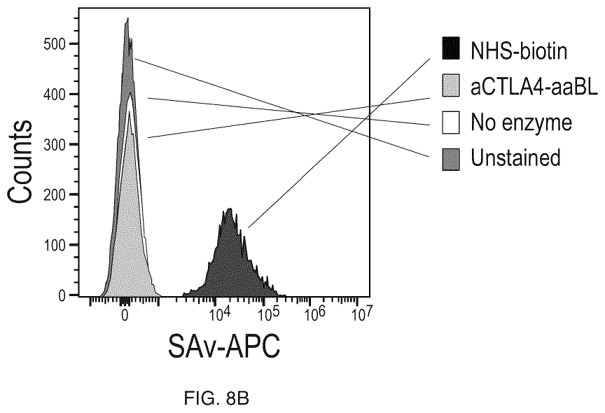

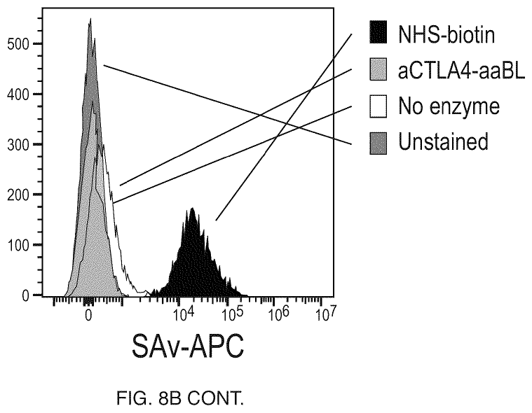

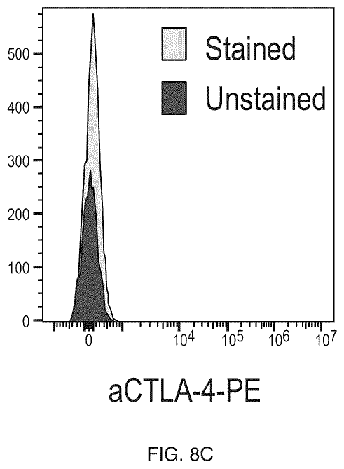

With isolated, functional aaBL and nanobody-aaBL fusions, their labeling activity was investigated on intact, living cells. Biotinylation was first assessed in an untargeted manner, i.e., with the enzyme alone or with the nanobody enzyme fusion in cells without the corresponding target marker, to confirm whether labeling occurs and to determine a baseline activity. B3Z cells, an immortalized T cell lymphoma line, were treated with 10 μM of the aaBL construct, 500 μM biotin, and 5 mM ATP for either 2 or 24 hours and then stained with streptavidin to assess biotinylation. While no change was observed after a 2 hour treatment, treating for 24 hours yielded a detectable increase in surface biotinylation for cells treated with the aCTLA-4 nanobody-aaBL fusion, having a ˜8-fold increase in the % streptavidin positive cells (FIGS. 8A-8B). Note that this cell line does not endogenously express CTLA-4, so treatment with this construct represents non-targeted baseline labeling (FIG. 8C). This data served as a critical foundation by establishing that enzymatic cell surface labeling occurred with the above enzymes and established parameters for detectable non-targeted labeling.

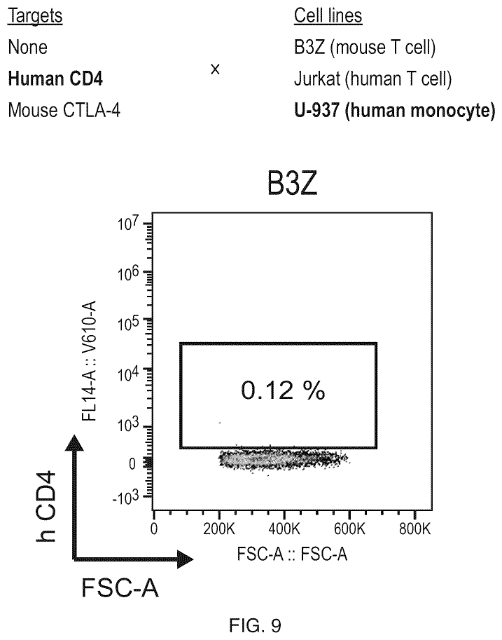

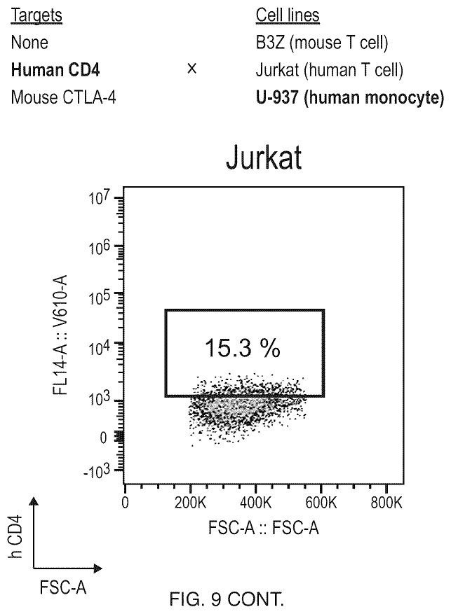

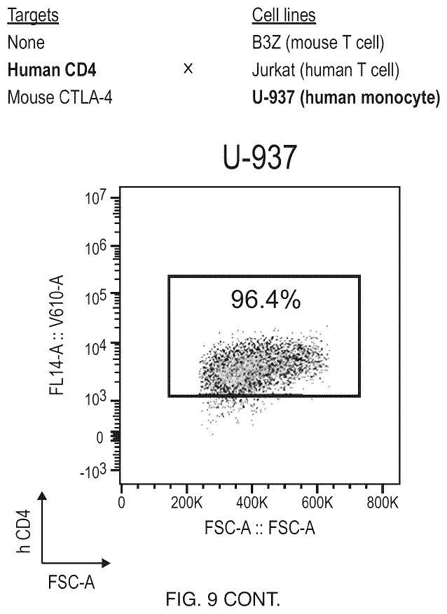

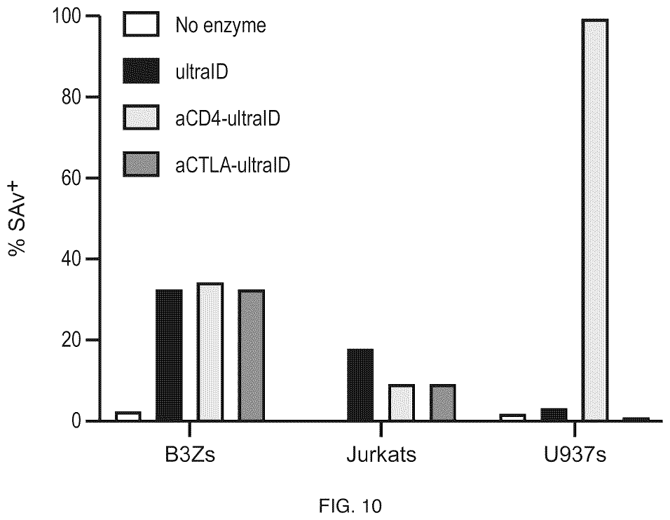

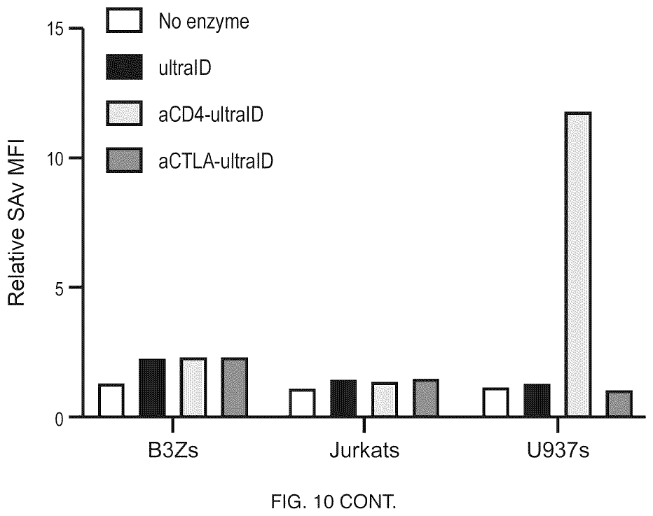

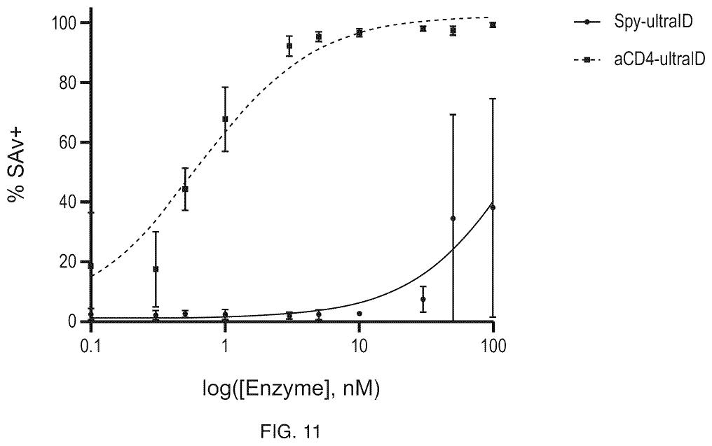

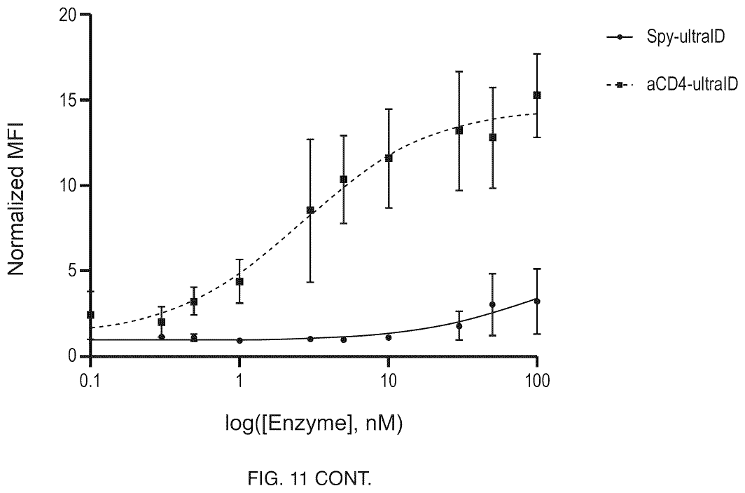

The targeting efficiency of this system was explored while also seeking to improve the overall activity of the enzymatic labeling. Initial steps involved performing a similar pilot experiment to that shown in FIGS. 8A-8C using B3Z and/or primary murine T cells induced to express CTLA-4. Further, targeted ligase selectively was shown to label cells based on surface marker expression with minimal off-target or background labeling (FIGS. 9, 10, and 11). For example, a construct comprised of the ligase fused to an anti-CD4 nanobody exhibited low nM/high pM efficiency, with an EC₅₀ of ˜0.6 nM. Comparing this biotin labeling with direct staining by the nanobody-enzyme fusion itself suggested that our targeted enzyme significantly amplifies labeling over the nanobody alone, a requirement for designing a targeting system with enhanced sensitivity. Even more effective targeting ligands could improve this sensitivity even further. For example, tumor markers are expressed by a wide range of tumor cells including EpCAM, CD47, CD44, and CEA. An initial study in mice using CD47 is being evaluated as to how well this enzyme-based targeting system performs in vivo since CD47 is expressed across cell types and over expressed by many tumors and tumor models, nanobodies have been identified against mouse and human CD47, and CD47 provides an inhibitory signal to macrophage envelopment. This system is being expanded to a range of targeting domains, which can be accomplished by expressing aaBL fused to range of nanobodies and other targeting domains; however, to streamline this process a construct comprising aaBL fused to the third generation SpyCatcher domain was expressed, which allowed for more rapid ligation to various targeting ligands and expanded the scope to also include nucleic acid-based ligands, e.g., aptamers. A handful of other enzymes were expressed to test, beginning with a monoglycine-reactive variant of sortase A. Subtiligase and several recently reported truncated variants of aaBL are being tested, including one evolved to increase its activity. In this manner, enzyme(s) are being identified with optimal activity. The already generated aaBL constructs have been used to design the two-component and TCR-targeting systems.

An alternative strategy is to implement a two-marker requirement into this targeting system based around protease activation. This approach appears promising, particularly considering the origin of the optimal biotin ligase and the dramatic difference in its activity in the presence and absence its native C-terminal domain. Now that the activity and selectivity of our targeted enzyme system were confirmed, a version of this system with a two-marker requirement is being implemented, to afford greater precision in targeting precise populations of cells. Further, the system's broad utility is being evaluated by implementing it in vivo and incorporating different targeting ligands and cargo molecules, particularly cytotoxic agents. As such, a system capable of selective ablation of a range of cell types is being created.

### Example 3: ATP-Dependent Biotin Protein Ligase Successfully Targeted Cancer Cells

Cancer cells' ability to stay hidden from the immune system and rapid growth makes it one the most difficult diseases to cure. Current anticancer therapies are very toxic to the body since it kills both cancer cells and healthy cells. Targeted anticancer therapies have the potential to more effectively attack cancer cells with reduced toxicity, but they are limited by the similarity in surface marker expression between cancer cells and healthy cells. Most cancer biomarkers are also expressed to some extent in healthy cells, where the anticancer therapies can cause side effects such as rash, cardiac dysfunction, thyroid dysfunction, hypertension, bleeding, and other chemotherapeutic side effects.

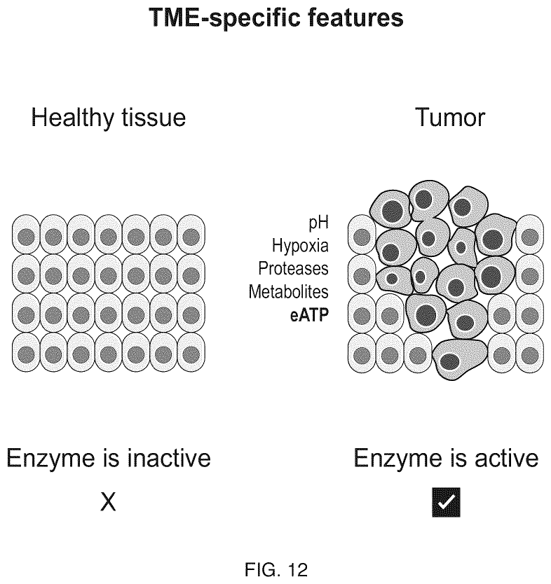

Although there are many similarities between cancer cells and healthy cells, there are many differences between the tumor microenvironment (TME) and normal tissue. Within the tumor microenvironment, there is cellular stress, tissue damage, hypoxia, and inflammation, and pH levels also differ in the tumor microenvironment (FIG. 12). One distinguishing feature of tumors is that the tumor microenvironment has elevated levels of extracellular ATP (˜1000-fold compared to healthy tissue). Using ATP-dependent enzymes allowed facilitation of cancer cell targeting. Biotin ligases (when modified to eliminate substrate specificity) use a molecule of ATP to attach a biotin molecule to nearby proteins. Zhao et al. developed a variant, UltraID, that is smaller and more efficient than other biotin ligases.

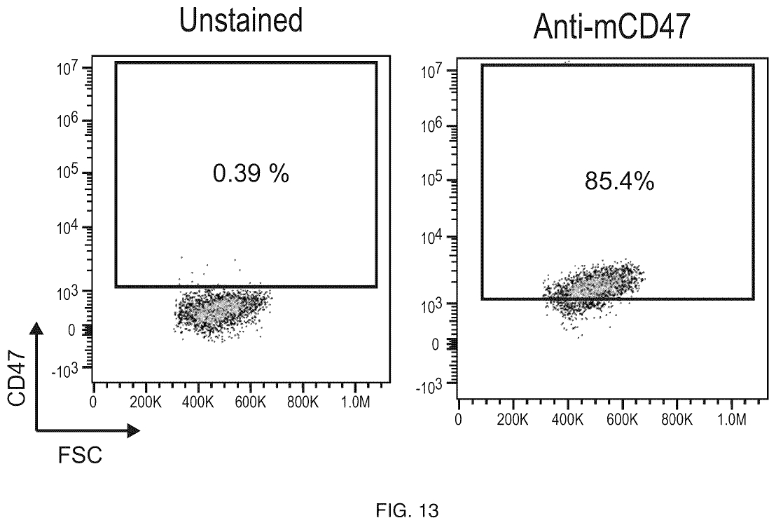

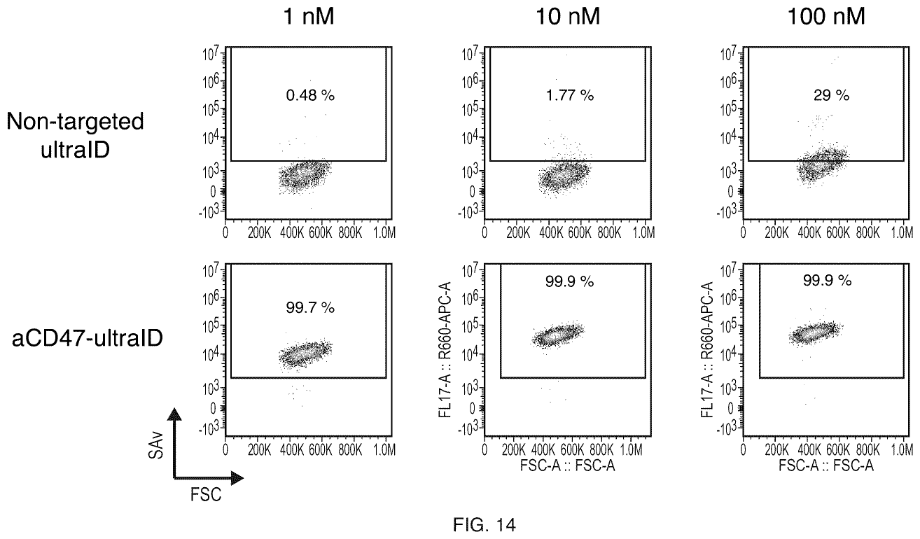

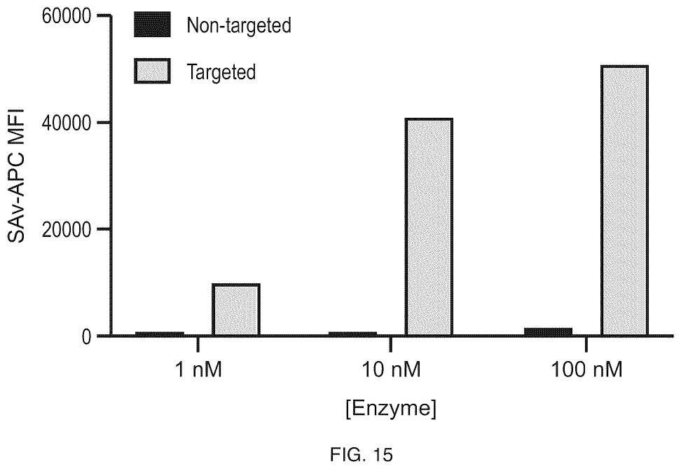

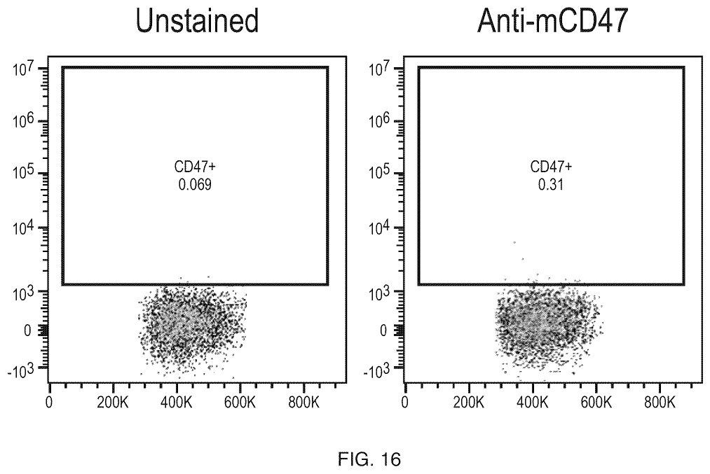

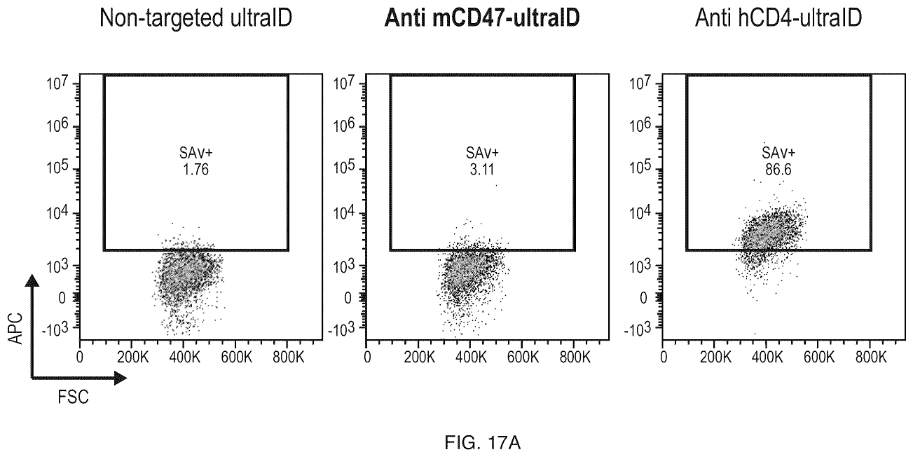

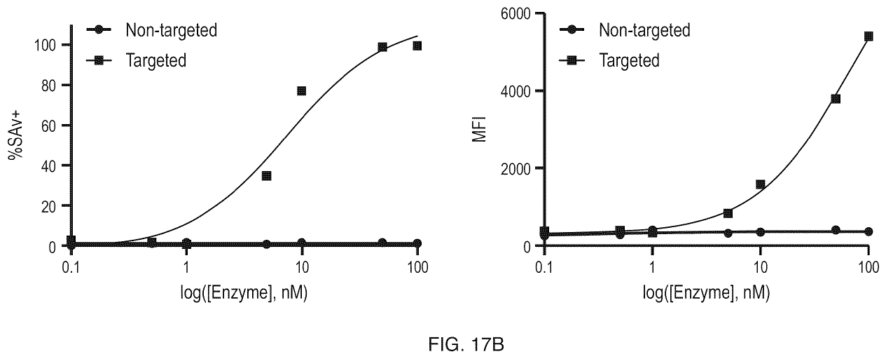

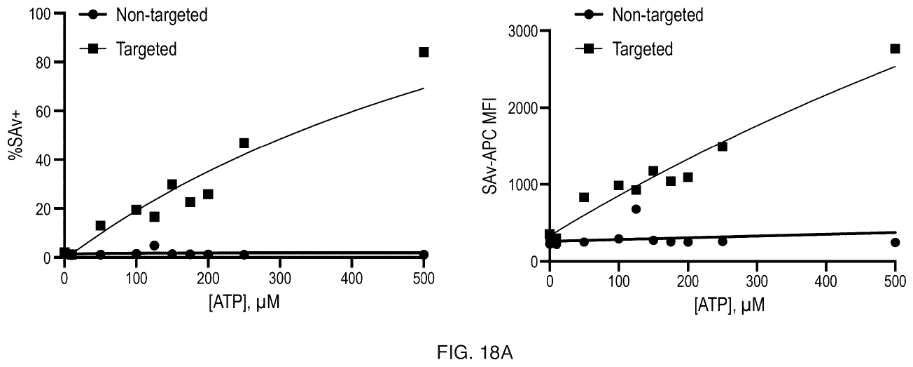

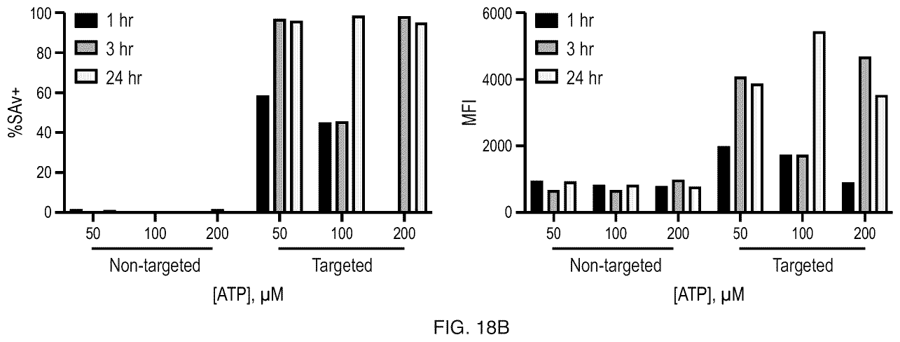

A nanobody against a cancer cell biomarker CD47 was attached to the UltraID enzyme so that upon binding, it biotinylated the surface of the target cell. FIG. 13 shows that B16 cells are CD47′ and can serve as an initial test case for tumor cell targeting. CD47 is a surface cancer cell biomarker that signals circulating immune cells to not eat them Since extracellular ATP is highly regulated in normal tissue, the ATP-dependent enzyme would only label cells within the tumor. FIG. 14 shows that B16 cells were treated with the indicated concentration of aCD47-ultraID, 50 μM biotin, and 1 mM ATP for 1 h at 37° C. then stained and analyzed via flow cytometry. aCD47-ultraID efficiently targeted b16 cells with near-complete labeling even at 1 nM. FIG. 15 shows targeted-ultraID exhibited a 10-fold increase in biotin labeling over the non-targeted ultraID at 1 nM treatment, with targeted activity increasing further at 10 and 100 nM. FIG. 16 shows stained U-937s with anti mCD47-AF488 antibody for 1 h at 4° C. U-937s do not appear to be stained by an anti mCD47 antibody and can serve as a negative control for aCD47-ultraID targeting. FIG. 17 shows that U-937s (which do not express mCD47) were treated with 10 nM of the indicated ultraID construct and analyzed. Note that aCD4-ultraID represents a positive control as U-937s are hCD4+. aCD47-ultraID did not biotinylate U-937s, confirming that its activity is specific to mCD47+ cells and represents successful targeting based on a tumor marker. FIG. 18 shows that B16 cells were treated with 25 nM of either non-targeted or aCD47-ultraID, 50 μM biotin and the indicated concentration of ATP for 1 hour at 37° C. then analyzed via flow cytometry. aCD47-ultraID is active at ATP concentrations typically found in the TME. The biotin molecules act as a label to distinguish cancer cells from healthy cells that can be used for targeted therapy, identification, and diagnostics.

### Example 4: Confirming Selectivity and Sensitivity of Enzyme-Based Targeting System

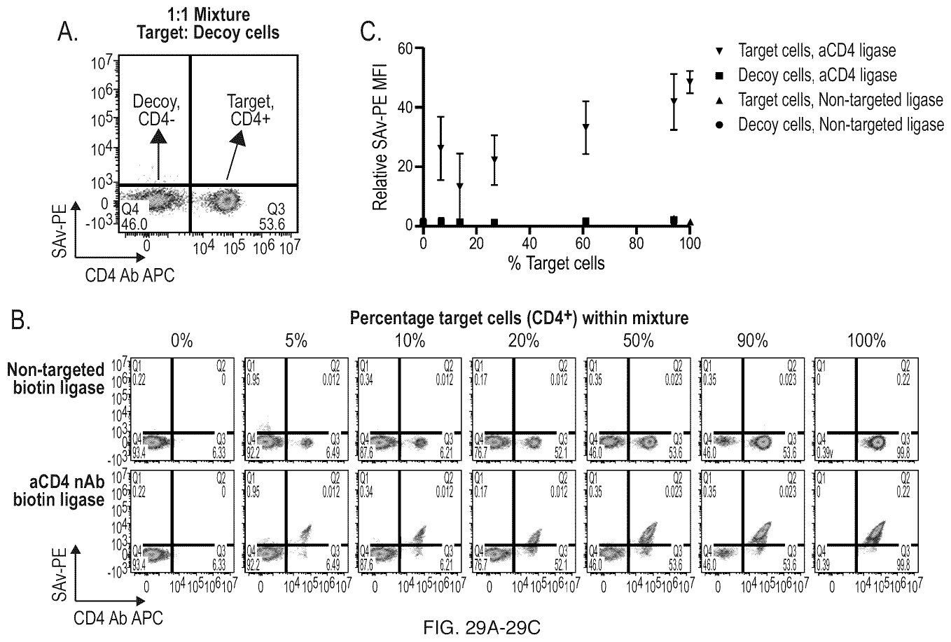

The enzyme-based targeting system uses a modified biotin ligase conjugated to a targeting domain (e.g., nanobody, antibody, fragments thereof, or other capture molecule) against a cell surface marker to biotinylate the surface of cells expressing the marker of interest, thereby introducing a convenient label that can be followed with a streptavidin or anti-biotin antibody reagent to accomplish a variety of objectives. This approach was expected to preserve the selectivity of the original targeting domain, with the radius of biotin labeling estimated to be 10-20 nm. This hypothesis was validated using an anti-hCD4 nanobody to target a population of CD4⁺ cells (U937s) mixed with a population of non-target cells (Jurkats) across a range of target: non-target ratios (FIG. 29A). Whether the percentage of target cells was 5% or 90%, only the intended target cells were labeled with biotin (FIG. 29B). The efficiency of on-target biotin labeling decreased slightly at lower target cell percentages but was relatively stable (FIG. 29C).

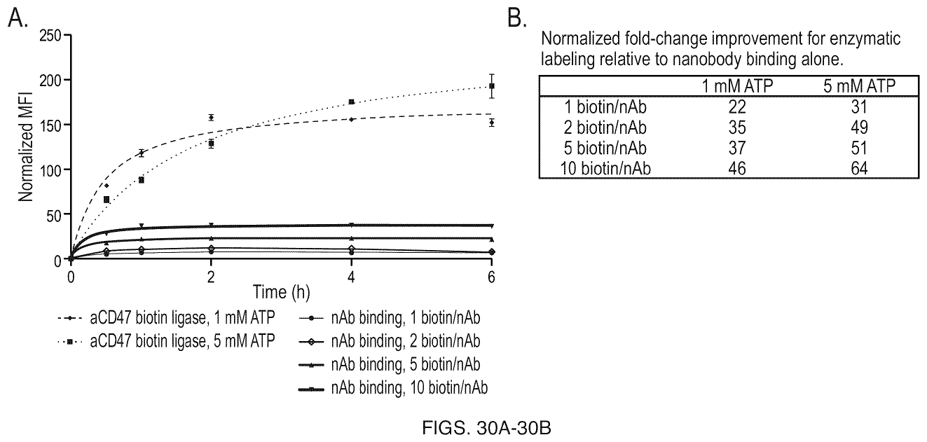

By using an enzyme, the system amplified the marker-specific signal and improved sensitivity over conventional targeting methods. This approach is non-destructive: mild treatment conditions with non-toxic substrates (ATP, biotin) avoids the damage to target cells incurred with harsher reagents like hydrogen peroxide. The sensitivity of targeted biotin ligase labeling was evaluated using B16 cells and an anti-mCD47 nanobody. Biotin labeling by the nanobody-enzyme conjugate was compared to an analogous nanobody construct that was itself biotinylated (using a biotin-NHS ester) at different stoichiometric ratios of biotin: nanobody construct. In this manner, streptavidin-PE could be used as a readout to compare simple nanobody binding vs. targeted enzymatic labeling. Enzyme-based labeling was significantly more efficient than binding by the nanobody construct alone, with at least a 20-fold increase in sensitivity (FIGS. 30A-30B). Note that the calculated fold-change increases appear to increase with biotin molecules/nAb, likely representing inefficiency in the NHS-biotin conjugation; even with this potential incomplete NHS-biotin reaction, an order of magnitude increase in sensitivity was observed with enzyme-based targeting (FIG. 30B). It is also worth noting that whereas direct labeling by the nanobody reached its maximum within about an hour, enzymatic labeling continued increasing for an additional hour or more (depending on the concentration of ATP used) as would be expected for a catalytic process vs. a simple binding interaction.

### Example 5: Evaluation of Optimal Treatment Conditions for Enzyme-Based Targeting

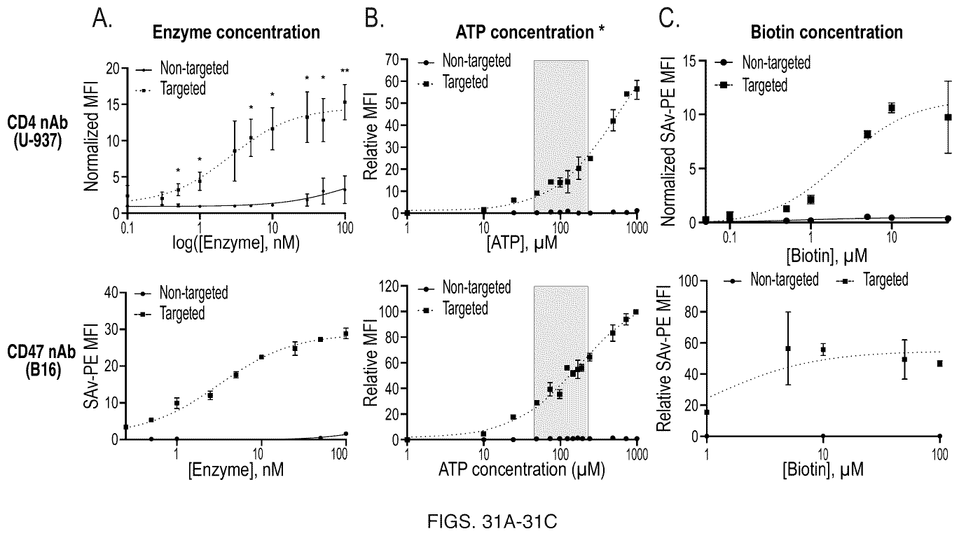

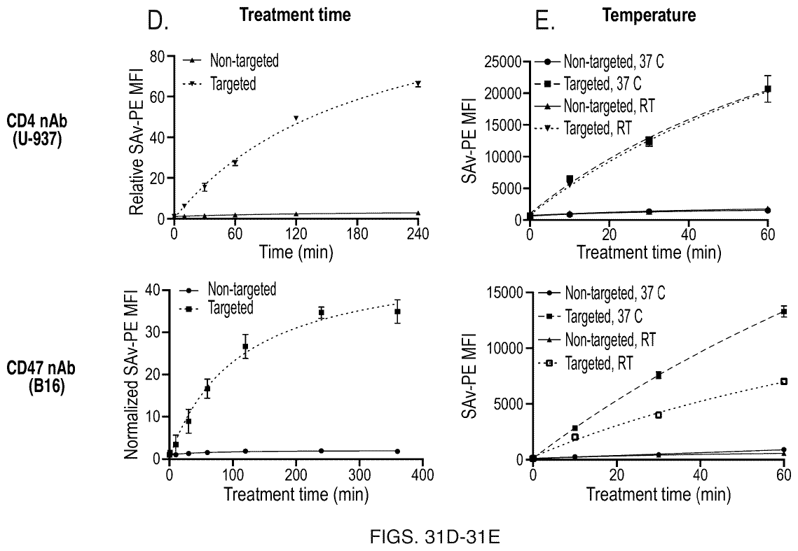

Optimal treatment conditions for enzyme-based targeting were determined in this Example, including enzyme concentration, substrate concentration, treatment time, and temperature. These experiments were conducted using two cell types and two target surface proteins as model systems (CD4 in a human myeloid cell line, U937, and CD47 in B16 murine melanoma cells). It was determined that enzymatic targeting (over a 1 h duration at 37° C.) occurs efficiently at low- to mid-nM concentrations of enzyme, mid-μM concentrations of biotin and mid-μM to low-mM concentrations of ATP (FIGS. 31A-31C). Importantly, the system retained activity at ATP concentrations typically found in the tumor microenvironment (50-200 μM, FIG. 31B). This provides a route to further enhance selectivity, for example to target tumor or tumor-resident/infiltrating cells, by taking advantage of biotin ligase's ATP dependence to constrain its activity to this physiological niche.

Enzymatic targeting efficiency over time was then evaluated using 25 nM biotin ligase, 50 μM biotin and 1 mM ATP. Targeted biotinylation was detected after as little as 30 minutes, reaching a maximum around 4-6 hours (FIG. 31D). Comparing targeted biotin labeling at room temperature and 37° C. over an hour treatment demonstrated that little to no activity was lost when treating at room temperature (FIG. 31E).

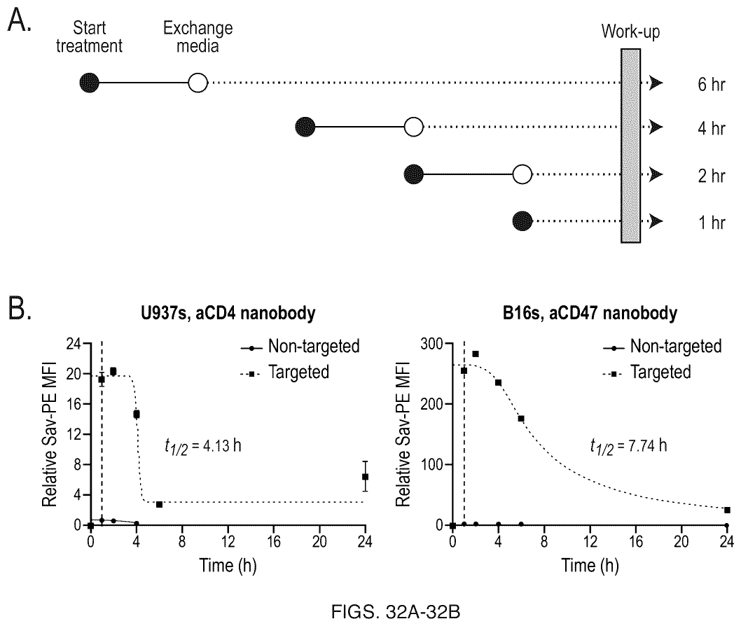

Next, it was determined how long the enzymatically-attached biotin was retained at the cell surface. Cells were treated with 25 nM targeted biotin ligase (or a non-targeted control), 50 μM biotin and 1 mM ATP for one hour, then exchanged for fresh media (no enzyme, biotin or ATP) and incubated for an additional 0-23 hours before measuring cell surface biotin labeling (FIG. 32A). The results indicated that while there was some variability between different cell types, enzymatically-attached biotin was efficiently retained at the cell surface for several hours after labeling, with a decrease in signal at 3-5 hours post-treatment but some signal still detected 23 hours after the targeted biotin ligase treatment ended (FIG. 32B).

### Example 6: Utilization of Commercial Antibodies with Enzyme-Based Targeting System to Target a Variety of Cell Types

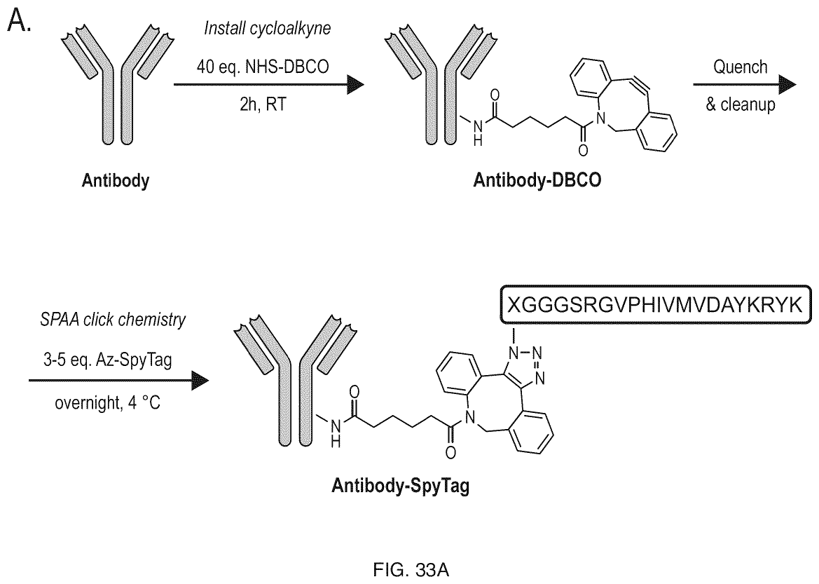

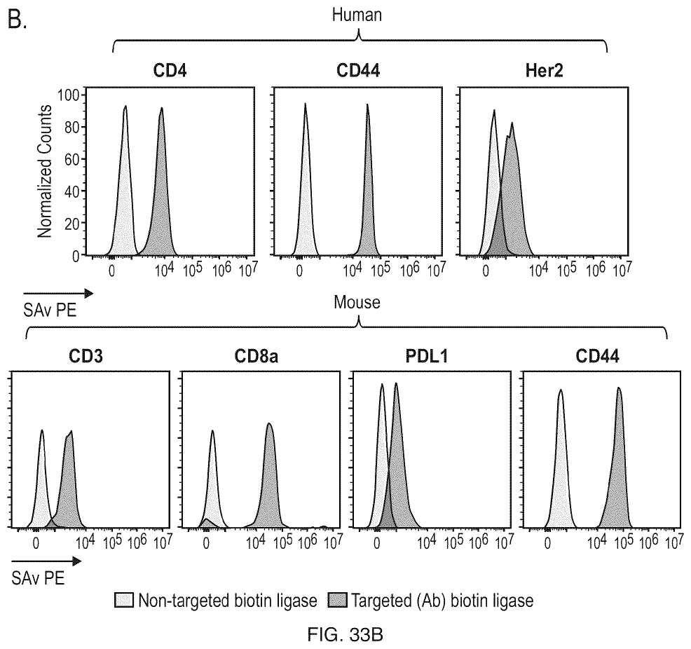

Building on nanobody-based targeting, the use of commercially-available antibodies was evaluated for the enzyme-based targeting system. Strain-promoted azide/alkyne click-chemistry was used to attach a peptide sequence (‘SpyTag’) to commercial antibodies to enable simple and rapid conjugation to the biotin ligase in the targeting system, which is expressed as a fusion with the corresponding ‘SpyCatcher’ domain (FIG. 33A). The SpyCatcher/SpyTag system was also used to conjugate the biotin ligase to the nanobodies used in previous experiments, with the difference being that the SpyTag sequence was fused to the nanobody. Without intending to be bound by theory, it is expected that other bioconjugation strategies could be used in place of this particular click chemistry approach to attach biotin ligase, if desired. This modular approach allowed for straightforward and general conjugation of a variety of commercial antibodies to biotin ligase, including antibodies targeting human CD4, CD44, and Her2 as well as murine CD3, CD8a, CD44 and PD-L1. These antibody-biotin ligase conjugates were evaluated in a variety of cell types, including immortalized human breast cancer, human myeloid, and murine melanoma cell lines as well as primary murine tumor-infiltrating lymphocytes (TILs) and OT-I CD8⁺ T cells. As expected, these antibody-biotin ligase conjugates efficiently and selectively labeled cells expressing their target markers (FIG. 33B). In particular, enzyme-based targeting of Her2 on Her2⁺ human breast cancer cells may provide more sensitive Her2-targeting therapies.

### Example 7: Targeted Biotin Ligase Based Detection Reagents

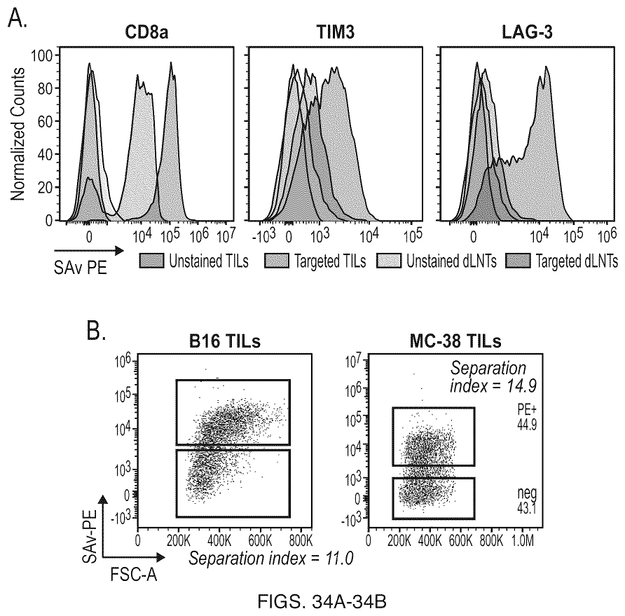

This versatility suggests a use for targeted biotin ligase as a non-destructive enzyme-based detection reagent, allowing more sensitive/precise detection, especially for challenging markers. For example, many exhaustion markers can be challenging to stain and distinguish in practice, so clear and straightforward detection in a non-destructive manner could streamline different experiments. Enzyme-based targeting was used to detect several markers in tumor-infiltrating lymphocytes isolated from tumors grown and removed from mice. CD8+ cells were isolated from excised B16 tumors or tumor-draining lymph nodes, then treated with an antibody-biotin ligase targeting CD8 (as a positive control), TIM-3 and LAG-3. Expression of all three markers was detected, distinguishing between expression levels in tumor-infiltrating T cells vs. T cells from the draining lymph node (FIG. 34A). Further, targeted biotin ligase labeling allowed for the effective distinguishing of LAG-3+ from LAG-3/LAG-310 cells in TILs isolated from both B16 and MC-38 tumors (FIG. 34B).

The following materials and methods were used in the Examples described herein.

### Biotin Ligases

The enzymes tested were biotin ligases from E. coli and A. aeolicus with an active site R→G point mutation to render them non-specific (so that they biotinylate any proximal lysine residues). These non-specific variants are termed BioID and BioID2 in the proximity labeling literature they were obtained from. Specifically, an optimized version of the A. aeolicus biotin ligase was used called ultraID that is truncated (K171) and has an additional active site point mutation (L->P). The ultraID variant is substantially more active than the precursor (BioID2) and is the core of the system in the experiments. All biotin ligases were produced in-house.

### Targeting Domains

Targeting domains tested include previously reported nanobodies as well as commercially available antibodies against various human and mouse tumor or immune markers (hCD4, hCD44, hHer2, mCD47, mCTLA-4, mCD3, mCD8a, mPD-L1, mTIM-3, and mLAG-3). Nanobodies were produced in-house and antibodies were purchased from Biolegend in an ultra-pure, azide free format (except aHer2 trastuzumab biosimilar, which was purchased from Leinco Technologies).

### Other Materials

Heterobifunctinal linkers comprised of dibenzocyclooctyne (DBCO) and N-hydroxysuccinimide (NHS) ester separated by a PEG4 linker for spacing and solubility were purchased from Lumiprobe and Sigma (‘NHS-DBCO’). Synthetic SpyTag peptide was ordered from Genscript with an azidoornithine residue at the N-terminus for click conjugation.

### Protein Expression and Isolation

Standard recombinant E. coli expression and affinity purification (His tag) techniques were used to produce nanobodies and ultraID fusions. Expression was induced with 50 μM IPTG once cultures had reached an OD600 of 0.6-0.8, then grown overnight at 18 or 22° C. Bacteria were pelleted, lysed, and protein was isolated using Co- or Ni-NTA resin. Isolated protein was then buffer exchanged into 50 mM Tris 150 mM NaCl, pH 8.0, flash-frozen in single-use aliquots and stored at −80° C.

UltraID was expressed as a fusion protein with SpyCatcher and the nanobodies were expressed with the Spy Tag peptide. These constructs could also be produced using a different conjugation system or expressed as a single fusion protein.

### Antibody-SpyTag Preparation

SpyTag peptide was conjugated to commercial antibodies to enable conjugation to the biotin ligase (via SpyCatcher fusion protein) using strain-promoted azide/alkyne click chemistry (‘copper-free click chemistry’). In the first step, dibenzocyclooctyne was installed on the antibody using a heterobifunctinal linker containing an N-hydroxysuccinimide ester. Antibody (1 equiv.) and NHS-DBCO (40 equiv.) were reacted in PBS at room temperature with gentle mixing for 2-3 hours, then the reaction was quenched with 80 mM Tris, pH 8.0 for 20 minutes. Buffer was exchanged and unreacted NHS-DBCO was removed using spin de-salting columns. The reaction product, antibody with DBCO functional groups installed, was reacted overnight at 4° C. with synthetic SpyTag peptide with an N-terminal azide (4 equiv.). Excess SpyTag peptide was removed and buffer was exchanged with spin de-salting columns. Antibody-Spy Tag product was stored at 4° C. in phosphate-buffered saline with 0.06% sodium azide.

### Preparation of Targeted Biotin Ligase Conjugates

Frozen aliquots of SpyCatcher-biotin ligase were thawed on ice (new aliquot for each experiment). SpyTag-nanobody/antibody aliquots were thawed on ice or obtained from 4° C. storage, as needed. Separate conjugation reactions were prepared for each nanobody/antibody by combining equimolar SpyCatcher-biotin ligase and SpyTag-nanobody/antibody in PBS to final concentration of 1-2.5 μM each. (Concentration and Ab: biotin ligase stoichiometry can be adjusted as desired, ex. for antibodies with >1 copy of DBCO per antibody.) Reactions were incubated at room temperature for 10 minutes to allow reaction to proceed to completion before proceeding with targeting experiment. Optionally, conjugation can be confirmed by SDS-PAGE.

### Enzymatic Labeling of Target Cells In Vitro

Cells (primary or immortalized line) were counted and plated as appropriate in a suitable media. Antibody/nanobody biotin ligase constructs were prepared prior to experiment as described above. Treatment media for each condition was prepared with biotin, ATP, and biotin ligase constructs. Unless otherwise stated, 50 μM biotin and 1 mM ATP were used. Typically, experiments used 10 or 25 nM biotin ligase. Cells were centrifuged for 4 min at 300×g (for suspension cells) or aspirated (adherent) to remove media and treatment media was applied. Cells were treated for 1-2 hours at 37° C. (for some experiments, this was extended to 6 hours or longer or performed at room temperature). After treatment, cells were trypsinized (adherent) and/or pelleted by centrifugation, transferred to a U-bottom 96-well plate and washed 2× with cell staining buffer. Cells were re-suspended in staining buffer with fluorochrome streptavidin and incubated at 4° C. for 20 min (0.1 μg APC-Streptavidin or PE-Streptavidin in 50 μL staining buffer; titrate time and concentration as needed). For some experiments, cells were co-stained with antibody fluorochrome to detect additional markers. Cells were washed 2× with staining buffer then re-suspended in staining buffer with live/dead stain (ex. SyTox Blue, 1:1000 dilution) and incubated at 4° C. for 3-5 min. Cells were analyzed via flow cytometry to evaluate biotinylation of the target cell population.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A fusion polypeptide comprising a biotin protein ligase comprising or consisting of an active site that has at least 85% amino acid sequence identity to the following sequence: GRGX₁X₂GRKW (SEQ ID NO: 2)
having biotin ligase activity; and
a targeting moiety fused to the biotin protein ligase polypeptide or fragment thereof at the N- or C-terminus,
wherein X₁ is G or S, and
wherein X₂ is P, L, or R.

2. The fusion polypeptide of claim 1, wherein the biotin protein ligase has at least 85% amino acid sequence identity to
(SEQ ID NO: 1)
MFKNLIWLKEVDSTQERLKEWNVSYGTALVADRQTKGRGGPGRKW
LSQEGGLYFSFLLNPKEFENLLQLPLVLGLSVSEALEEITEIPFS
LKWPNDVYFQEKKVSGVLCELSKDKLIVGIGINVNQREIPEEIKD
RATTLYEITGKDWDRKEVLLKVLKRISENLKKFKEK.

3. The fusion polypeptide of claim 1, wherein the variant polypeptide is derived from E. coli, or A. aeolicus.

4. A polynucleotide encoding the fusion polypeptide of claim 1.

5. The fusion polypeptide of claim 1, wherein the fusion polypeptide further comprises a blocking domain that inhibits the polypeptide ligase activity.

6. The fusion polypeptide of claim 5, wherein the blocking domain comprises at least a fragment of a wild-type biotin protein ligase.

7. The fusion polypeptide of claim 6, wherein the C-terminal domain of the blocking domain has at least 85% amino acid sequence identity to the following amino acid sequence:
(SEQ ID NO: 3)
ENLYFQGSFKEFKGKIESKMLYLGEEVKLLGEGKITGKLVGLSEK
GGALILTEEGIKEILSGEFSLRRSGGS.

8. The fusion polypeptide of claim 7, wherein the blocking domain is linked to the biotin protein ligase variant by a cleavable linker.

9. The fusion polypeptide of claim 8, wherein the cleavable linker comprises a protease recognition sequence targeted by a furin, Tobacco Etch Virus (TEV), Rhinovirus 3C, Enterokinase, Factor Xa or other protease.

10. The fusion polypeptide of claim 1, wherein the polypeptide further comprises a flexible spacer sequence that comprises one or more of the following sequences: GGGS (SEQ ID NO: 4), GGGGG (SEQ ID NO: 5), GSGSGS (SEQ ID NO: 6), GGSGGS (SEQ ID NO: 7), GGGGS (SEQ ID NO: 8), GGGGSLVPRGSGGGGS (SEQ ID NO: 9), GGSGGHMGSGG (SEQ ID NO: 10), VEGGSGGSGGSGGSGGV (SEQ ID NO: 11), and GSTSGSGXPGSGEGSTKG (SEQ ID NO: 12).

11. The fusion polypeptide of claim 1, wherein the fusion polypeptide further comprises a Spycatcher domain or chaperone domain.

12. A method of targeting a cell type of interest, the method comprising contacting the cell with the fusion polypeptide of claim 1 under conditions that support ligase activity.

13. The method of claim 12, where in the contacting is carried out in the presence of exogenous or endogenous_ATP.

14. The method of claim 12, wherein the contacting is carried out in the presence of exogenous biotin.

15. A method of labeling a cell, the method comprising contacting the cell with a biotin binding moiety covalently linked to a detectable moiety and a fusion polypeptide under conditions that support ligase activity, the fusion polypeptide comprising
i) an agent that specifically binds the cell; and
ii) a biotin protein ligase comprising an active site that has at least 85% amino acid sequence identity to the following sequence:
(SEQ ID NO: 2)
GRGX₁X₂GRKW
and having biotin ligase activity,
wherein X₁ is G or S, and
wherein X₂ is P, L, or R, thereby labeling the cell.

16. A method of delivering an agent to a cell, the method comprising contacting a cell with a fusion polypeptide of claim 1 under conditions that support ligase activity, and a biotin binding moiety covalently linked to an agent, thereby delivering the agent to the cell.

17. The method of claim 16, wherein the cell is a tumor cell, and wherein the tumor cell is within a tumor microenvironment (TME).

18. The method of claim 16, wherein the TME is characterized by the presence of an ATP concentration of from about 10 μM to about 1 mM.

19. A method for producing the fusion protein of claim 1, the method comprising expressing the fusion protein in a cell.

20. A kit comprising the fusion polypeptide of claim 1, and directions for the use of the kit and the methods described herein.

21. A system for use in delivering an agent to a cell of interest, the system comprising a fusion protein comprising a biotin protein ligase fused to a targeting moiety, biotin, ATP, and a biotin binding moiety fused to an agent for delivery to the cell.
